SEQUENCE LISTING (240 sequences)

SEQ ID NO: 1 - DNA, 1569 bp, Homo sapiens
  source 1..1569: mol_type = genomic DNA
ctgacaatcgttgtgcagttgatgtccacattaaaatactggattttcccacgttagaagaatgtttaaatttagtatatgtgggacaaagtggaagacacacagatttatacatgcacatacttttcttcattcacttctttgtacttaagtttaggaatcttcccacttacagatggataaatgggtacaatgaagggccaatagccctccctgtctgtattgagggtgtgggtctctaccttgggtgctgttctctgcctcaggagctctctgtcaattgcaggagcctctgaggagaaaattgacctttcttggctggggcagagaacatacggtatgcagggttcaggctcctgacggagttggggcaaccctggagataagctcacacaaccctgcaagaccaggtgctgttaccctagccaatctcatggatgaaccagatcaatgccagatgagctctgcctaaaatgattttttggtgaactctgaaaagtggaatattgtttctgtaagaatatccatctgagactctatctcttggtaataccaaccaagagttatcagtttctctttaaccgagacaccagcaaagtgcctgctccagggtactgcccaggggagccctccatttgtagaatgaatgagagtccaggttatgaacagtgcctggagtgtaggaacaccctcctttgcctctttgacaggtctgcatcataacactttttttttttttttgagacagagtctcactctgtcgcccaggctggagtgcagtggcacgatctcggccccctgcaagttccgcctcccgggttcacaccattctcctgcctcagcctccccagcagctgggactacaggcacctgccgccacgcccggctaattttttgtatttttagtagagacagggtttcaccatgttagccaggatggtctcgatctcctgaccttgtgatctgcccgcctcggcctcccaaagtgttgggattacaggcgtgagccaccgtgtccagcctgtaacacttcttatagcactgagttgaaaccttgctcctcctggttcctccaggaaactgaaatctttttgagccaagtctagcacagtgcctggcatgtacattcaggtggtagagtttgctgcttgaatgggtgaatgggaatttgacagcatttttattcaaattagtatgtgccaggtatcgtgctcgctctgcattatccaagggagtgagcctctgtgcaagtatttgagacacgagggaaataggttctactgtgggaaaaagagcatttcatggacttgctctccaagcagccttctgatttttaatttggctcccagtatcttgatatcaggagtcagtcacaagaactccatctttagtaagttatattttccacaggaaatctaaaagctgttcaacatgttagtttcctgtgaatttgataagccataatccattcctaacactgagccctcctgaaatttggtgtctggtcctgcagatagctaaaagccctgtctgggtggcctaggggactcctctgttttgcctccacag

SEQ ID NO: 2 - DNA, 20 bp, synthetic construct
  source 1..20: mol_type = other DNA
  misc_feature 1..5: note = RNA
  misc_feature 16..20: note = RNA
  misc_feature 6..15: note = DNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
gaacttgcagggggccgaga

SEQ ID NO: 3 - DNA, 20 bp, synthetic construct
  source 1..20: mol_type = other DNA
  misc_feature 1..5: note = RNA
  misc_feature 16..20: note = RNA
  misc_feature 6..15: note = DNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
ccctcaatacagacagggag

SEQ ID NO: 4 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
acttttcagagttcaccaaa

SEQ ID NO: 5 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
tggactctcattcattctac

SEQ ID NO: 6 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
cttgcagggggccgagatcg

SEQ ID NO: 7 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
tttcagagttcaccaaaaaa

SEQ ID NO: 8 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
aaaaaaaaaaagtgttatga

SEQ ID NO: 9 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
cctggactctcattcattct

SEQ ID NO: 10 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
agcacgatacctggcacata

SEQ ID NO: 11 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
gagcacgatacctggcacat

SEQ ID NO: 12 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
atctggcattgatctggttc

SEQ ID NO: 13 - DNA, 20 bp, synthetic construct
  source 1..20: mol_type = other DNA
  misc_feature 1..5: note = RNA
  misc_feature 6..15: note = DNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
  misc_feature 16..20: note = RNA
catctggcattgatctggtt

SEQ ID NO: 14 - DNA, 20 bp, synthetic construct
  source 1..20: mol_type = other DNA
  misc_feature 6..15: note = DNA
  misc_feature 1..5: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
  misc_feature 16..20: note = RNA
gccaaattaaaaatcagaag

SEQ ID NO: 15 - DNA, 20 bp, synthetic construct
  source 1..20: mol_type = other DNA
  misc_feature 1..5: note = RNA
  misc_feature 6..15: note = DNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
  misc_feature 16..20: note = RNA
gcggaacttgcagggggccg

SEQ ID NO: 16 - DNA, 20 bp, synthetic construct
  source 1..20: mol_type = other DNA
  misc_feature 6..15: note = DNA
  misc_feature 16..20: note = RNA
  misc_feature 1..5: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
taaaaatacaaaaaattagc

SEQ ID NO: 17 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
atactaatttgaataaaaat

SEQ ID NO: 18 - DNA, 20 bp, synthetic construct
  source 1..20: mol_type = other DNA
  misc_feature 6..15: note = DNA
  misc_feature 16..20: note = RNA
  misc_feature 1..5: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
tactaatttgaataaaaatg

SEQ ID NO: 19 - DNA, 20 bp, synthetic construct
  source 1..20: mol_type = other DNA
  misc_feature 6..15: note = DNA
  misc_feature 16..20: note = RNA
  misc_feature 1..5: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
cagcttttagatttcctgtg

SEQ ID NO: 20 - DNA, 20 bp, synthetic construct
  source 1..20: mol_type = other DNA
  misc_feature 1..5: note = RNA
  misc_feature 16..20: note = RNA
  misc_feature 6..15: note = DNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
agcttttagatttcctgtgg

SEQ ID NO: 21 - DNA, 20 bp, synthetic construct
  source 1..20: mol_type = other DNA
  misc_feature 6..15: note = DNA
  misc_feature 16..20: note = RNA
  misc_feature 1..5: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
aagaagtgaatgaagaaaag

SEQ ID NO: 22 - DNA, 20 bp, synthetic construct
  source 1..20: mol_type = other DNA
  misc_feature 16..20: note = RNA
  misc_feature 6..15: note = DNA
  misc_feature 1..5: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
tacaaagaagtgaatgaaga

SEQ ID NO: 23 - DNA, 20 bp, synthetic construct
  source 1..20: mol_type = other DNA
  misc_feature 6..15: note = DNA
  misc_feature 16..20: note = RNA
  misc_feature 1..5: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
acaaagaagtgaatgaagaa

SEQ ID NO: 24 - DNA, 20 bp, synthetic construct
  source 1..20: mol_type = other DNA
  misc_feature 16..20: note = RNA
  misc_feature 6..15: note = DNA
  misc_feature 1..5: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
gttaaagagaaactgataac

SEQ ID NO: 25 - DNA, 20 bp, synthetic construct
  source 1..20: mol_type = other DNA
  misc_feature 6..15: note = DNA
  misc_feature 1..5: note = RNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
agacccacaccctcaataca

SEQ ID NO: 26 - DNA, 20 bp, synthetic construct
  source 1..20: mol_type = other DNA
  misc_feature 16..20: note = RNA
  misc_feature 6..15: note = DNA
  misc_feature 1..5: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
actaatttgaataaaaatgc

SEQ ID NO: 27 - DNA, 20 bp, synthetic construct
  source 1..20: mol_type = other DNA
  misc_feature 1..5: note = RNA
  misc_feature 16..20: note = RNA
  misc_feature 6..15: note = DNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
gcttttagatttcctgtgga

SEQ ID NO: 28 - DNA, 20 bp, synthetic construct
  source 1..20: mol_type = other DNA
  misc_feature 1..5: note = RNA
  misc_feature 16..20: note = RNA
  misc_feature 6..15: note = DNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
caaattaaaaatcagaaggc

SEQ ID NO: 29 - DNA, 20 bp, synthetic construct
  source 1..20: mol_type = other DNA
  misc_feature 6..15: note = DNA
  misc_feature 16..20: note = RNA
  misc_feature 1..5: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
ccaaattaaaaatcagaagg

SEQ ID NO: 30 - DNA, 20 bp, synthetic construct
  source 1..20: mol_type = other DNA
  misc_feature 6..15: note = DNA
  misc_feature 16..20: note = RNA
  misc_feature 1..5: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
tatcaaattcacaggaaact

SEQ ID NO: 31 - DNA, 20 bp, synthetic construct
  source 1..20: mol_type = other DNA
  misc_feature 1..5: note = RNA
  misc_feature 16..20: note = RNA
  misc_feature 6..15: note = DNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
ttatcaaattcacaggaaac

SEQ ID NO: 32 - DNA, 20 bp, synthetic construct
  source 1..20: mol_type = other DNA
  misc_feature 6..15: note = DNA
  misc_feature 16..20: note = RNA
  misc_feature 1..5: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
ctaatttgaataaaaatgct

SEQ ID NO: 33 - DNA, 20 bp, synthetic construct
  source 1..20: mol_type = other DNA
  misc_feature 1..5: note = RNA
  misc_feature 6..15: note = DNA
  misc_feature 6..15: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
ccaggaggagcaaggtttca

SEQ ID NO: 34 - DNA, 20 bp, synthetic construct
  source 1..20: mol_type = other DNA
  misc_feature 6..15: note = DNA
  misc_feature 6..15: note = RNA
  misc_feature 1..5: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
ctcatctggcattgatctgg

SEQ ID NO: 35 - DNA, 20 bp, synthetic construct
  source 1..20: mol_type = other DNA
  misc_feature 16..20: note = RNA
  misc_feature 1..5: note = RNA
  misc_feature 6..15: note = DNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
tgtgactgactcctgatatc

SEQ ID NO: 36 - DNA, 20 bp, synthetic construct
  source 1..20: mol_type = other DNA
  misc_feature 6..15: note = DNA
  misc_feature 1..5: note = RNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
gctcccctgggcagtaccct

SEQ ID NO: 37 - DNA, 20 bp, synthetic construct
  source 1..20: mol_type = other DNA
  misc_feature 6..15: note = DNA
  misc_feature 1..5: note = RNA
  misc_feature 6..15: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
ccaagagatagagtctcaga

SEQ ID NO: 38 - DNA, 20 bp, synthetic construct
  source 1..20: mol_type = other DNA
  misc_feature 6..15: note = DNA
  misc_feature 16..20: note = RNA
  misc_feature 1..5: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
ggaggagcaaggtttcaact

SEQ ID NO: 39 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
cttatcaaattcacaggaaa

SEQ ID NO: 40 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
catcaactgcacaacg

SEQ ID NO: 41 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
acatcaactgcacaac

SEQ ID NO: 42 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
ggacatcaactgcaca

SEQ ID NO: 43 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
tggacatcaactgcac

SEQ ID NO: 44 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
ttgtcccacatatact

SEQ ID NO: 45 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
actttgtcccacatat

SEQ ID NO: 46 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
cactttgtcccacata

SEQ ID NO: 47 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
ctaaacttaagtacaa

SEQ ID NO: 48 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
cctaaacttaagtaca

SEQ ID NO: 49 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
tgtacccatttatcca

SEQ ID NO: 50 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
accgtatgttctctgc

SEQ ID NO: 51 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
gggttgtgtgagctta

SEQ ID NO: 52 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
aatcattttaggcaga

SEQ ID NO: 53 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
aaatcattttaggcag

SEQ ID NO: 54 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
aaaatcattttaggca

SEQ ID NO: 55 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
acttttcagagttcac

SEQ ID NO: 56 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
cacttttcagagttca

SEQ ID NO: 57 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
ttccacttttcagagt

SEQ ID NO: 58 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
attccacttttcagag

SEQ ID NO: 59 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
aatattccacttttca

SEQ ID NO: 60 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
caatattccacttttc

SEQ ID NO: 61 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
acaatattccactttt

SEQ ID NO: 62 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
tctcagatggatattc

SEQ ID NO: 63 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
gtctcagatggatatt

SEQ ID NO: 64 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
agtctcagatggatat

SEQ ID NO: 65 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
gagtctcagatggata

SEQ ID NO: 66 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
agagtctcagatggat

SEQ ID NO: 67 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
tagagtctcagatgga

SEQ ID NO: 68 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
atagagtctcagatgg

SEQ ID NO: 69 - DNA, 16 bp, synthetic construct
  source 1..16: mol_type = other DNA
  misc_feature 1..3: note = RNA
  misc_feature 4..13: standard_name = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
accaagagatagagtc

SEQ ID NO: 70 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
taccaagagatagagt

SEQ ID NO: 71 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
ttaccaagagatagag

SEQ ID NO: 72 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
aactgcacaacgattg

SEQ ID NO: 73 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
caactgcacaacgatt

SEQ ID NO: 74 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
tcaactgcacaacgat

SEQ ID NO: 75 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
atcaactgcacaacga

SEQ ID NO: 76 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
gcccttcattgtaccc

SEQ ID NO: 77 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
gctattggcccttcat

SEQ ID NO: 78 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
ggctattggcccttca

SEQ ID NO: 79 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
agggctattggccctt

SEQ ID NO: 80 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
gagggctattggccct

SEQ ID NO: 81 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
ggagggctattggccc

SEQ ID NO: 82 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
acagggagggctattg

SEQ ID NO: 83 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
agcacccaaggtagag

SEQ ID NO: 84 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
cagcacccaaggtaga

SEQ ID NO: 85 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
gaaccctgcataccgt

SEQ ID NO: 86 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
tgaaccctgcataccg

SEQ ID NO: 87 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
cctgaaccctgcatac

SEQ ID NO: 88 - DNA, 16 bp, synthetic construct
  source 1..16: mol_type = other DNA
  misc_feature 1..3: note = RNA
  misc_feature 4..13: note = DNA
  misc_feature 14..16: note = DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
cccaactccgtcagga

SEQ ID NO: 89 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
agggttgccccaactc

SEQ ID NO: 90 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
ccagggttgccccaac

SEQ ID NO: 91 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
gattggctagggtaac

SEQ ID NO: 92 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
agattggctagggtaa

SEQ ID NO: 93 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
gagattggctagggta

SEQ ID NO: 94 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
atgagattggctaggg

SEQ ID NO: 95 - DNA, 16 bp, synthetic construct
  source 1..16: mol_type = other DNA
  misc_feature 1..3: note = RNA
  misc_feature 4..13: note = RNA
  misc_feature 14..16: note = DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
catgagattggctagg

SEQ ID NO: 96 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
ccatgagattggctag

SEQ ID NO: 97 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
ggtattaccaagagat

SEQ ID NO: 98 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
ttggtattaccaagag

SEQ ID NO: 99 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
gttggtattaccaaga

SEQ ID NO: 100 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
tggttggtattaccaa

SEQ ID NO: 101 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
cttggttggtattacc

SEQ ID NO: 102 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
taactcttggttggta

SEQ ID NO: 103 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
ataactcttggttggt

SEQ ID NO: 104 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
gataactcttggttgg

SEQ ID NO: 105 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
ccaagaaaggtcaatt

SEQ ID NO: 106 - DNA, 15 bp, synthetic construct
  source 1..15: mol_type = other DNA
  misc_feature 1..3: note = RNA
  misc_feature 4..12: note = DNA
  misc_feature 13..15: note = RNA
  modified_base 1..3: mod_base = OTHER; note = 2'-methoxyethyl (2'-MOE) modified nucleoside
  modified_base 13..15: mod_base = OTHER; note = 2'-methoxyethyl (2'-MOE) modified nucleoside
aacacgtctatacgc

SEQ ID NO: 107 - DNA, 15 bp, synthetic construct
  source 1..15: mol_type = other DNA
  misc_feature 1..3: note = RNA
  misc_feature 4..12: note = DNA
  misc_feature 13..15: note = RNA
  modified_base 1..3: mod_base = OTHER; note = locked nucleic acid (LNA) modified nucleoside (cMe; beta-D-methyleneoxy)
  modified_base 13..15: mod_base = OTHER; note = locked nucleic acid (LNA) modified nucleoside (cMe; beta-D-methyleneoxy)
aacacgtctatacgc

SEQ ID NO: 108 - DNA, 20 bp, synthetic construct
  source 1..20: mol_type = other DNA
  misc_feature 1..5: note = RNA
  modified_base 1..5: mod_base = OTHER; note = 2'-methoxyethyl (2'-MOE) modified nucleoside
  misc_feature 16..20: note = RNA
  modified_base 16..20: mod_base = OTHER; note = 2'-methoxyethyl (2'-MOE) modified nucleoside
  misc_feature 6..15: note = DNA
gtggacatcaactgcacaac

SEQ ID NO: 109 - DNA, 20 bp, synthetic construct
  source 1..20: mol_type = other DNA
  misc_feature 1..6: note = RNA
  misc_feature 7..14: note = DNA
  misc_feature 15..20: note = RNA
  modified_base 1..6: mod_base = OTHER; note = 2'-methoxyethyl (2'-MOE) modified nucleoside
  modified_base 15..20: mod_base = OTHER; note = 2'-methoxyethyl (2'-MOE) modified nucleoside
gtggacatcaactgcacaac

SEQ ID NO: 110 - DNA, 20 bp, synthetic construct
  source 1..20: mol_type = other DNA
  misc_feature 1..7: note = RNA
  misc_feature 8..13: note = DNA
  misc_feature 14..20: note = RNA
  modified_base 14..20: mod_base = OTHER; note = 2'-methoxyethyl (2'-MOE) modified nucleoside
  modified_base 1..7: mod_base = OTHER; note = 2'-methoxyethyl (2'-MOE) modified nucleoside
gtggacatcaactgcacaac

SEQ ID NO: 111 - DNA, 18 bp, synthetic construct
  source 1..18: mol_type = other DNA
  misc_feature 1..4: note = RNA
  misc_feature 5..14: note = DNA
  misc_feature 15..18: note = RNA
  modified_base 1..4: mod_base = OTHER; note = 2'-methoxyethyl (2'-MOE) modified nucleoside
  modified_base 15..18: mod_base = OTHER; note = 2'-methoxyethyl (2'-MOE) modified nucleoside
tggacatcaactgcacaa

SEQ ID NO: 112 - DNA, 18 bp, synthetic construct
  source 1..18: mol_type = other DNA
  misc_feature 1..5: note = RNA
  misc_feature 14..18: note = RNA
  misc_feature 6..13: note = DNA
  modified_base 1..5: mod_base = OTHER; note = 2'-methoxyethyl (2'-MOE) modified nucleoside
  modified_base 14..18: mod_base = OTHER; note = 2'-methoxyethyl (2'-MOE) modified nucleoside
tggacatcaactgcacaa

SEQ ID NO: 113 - DNA, 16 bp, synthetic construct
  source 1..16: mol_type = other DNA
  misc_feature 1..4: note = RNA
  misc_feature 13..16: note = RNA
  misc_feature 5..12: note = DNA
  modified_base 1..4: mod_base = OTHER; note = 2'-methoxyethyl (2'-MOE) modified nucleoside
  modified_base 13..16: mod_base = OTHER; note = 2'-methoxyethyl (2'-MOE) modified nucleoside
ggacatcaactgcaca

SEQ ID NO: 114 - DNA, 16 bp, synthetic construct
  source 1..16: mol_type = other DNA
  misc_feature 1..5: note = RNA
  misc_feature 12..16: note = RNA
  misc_feature 6..11: note = DNA
  modified_base 1..5: mod_base = OTHER; note = 2'-methoxyethyl (2'-MOE) modified nucleoside
  modified_base 12..16: mod_base = OTHER; note = 2'-methoxyethyl (2'-MOE) modified nucleoside
ggacatcaactgcaca

SEQ ID NO: 115 - DNA, 17 bp, synthetic construct
  source 1..17: mol_type = other DNA
  misc_feature 1..3: note = RNA
  misc_feature 14..17: note = RNA
  misc_feature 4..13: note = DNA
  modified_base 1..3: mod_base = OTHER; note = locked nucleic acid (LNA) modified nucleoside (cMe; beta-D-methyleneoxy)
  modified_base 14..17: mod_base = OTHER; note = locked nucleic acid (LNA) modified nucleoside (cMe; beta-D-methyleneoxy)
tggacatcaactgcaca

SEQ ID NO: 116 - DNA, 17 bp, synthetic construct
  source 1..17: mol_type = other DNA
  misc_feature 1..5: note = RNA
  misc_feature 14..17: note = RNA
  misc_feature 6..13: note = DNA
  modified_base 1..5: mod_base = OTHER; note = locked nucleic acid (LNA) modified nucleoside (cMe; beta-D-methyleneoxy)
  modified_base 14..17: mod_base = OTHER; note = locked nucleic acid (LNA) modified nucleoside (cMe; beta-D-methyleneoxy)
tggacatcaactgcaca

SEQ ID NO: 117 - DNA, 16 bp, synthetic construct
  source 1..16: mol_type = other DNA
  misc_feature 1..5: note = RNA
  misc_feature 13..16: note = RNA
  misc_feature 6..12: note = DNA
  modified_base 1..5: mod_base = OTHER; note = locked nucleic acid (LNA) modified nucleoside (cMe; beta-D-methyleneoxy)
  modified_base 13..16: mod_base = OTHER; note = locked nucleic acid (LNA) modified nucleoside (cMe; beta-D-methyleneoxy)
ggacatcaactgcaca

SEQ ID NO: 118 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 14..16: note = RNA
  misc_feature 4..13: note = DNA
  modified_base 6: mod_base = OTHER; note = 2'OMe modified deoxynucleoside
ggacatcaactgcaca

SEQ ID NO: 119 - DNA, 20 bp, synthetic construct
  source 1..20: mol_type = other DNA
  misc_feature 1..5: note = RNA
  misc_feature 16..20: note = RNA
  misc_feature 6..15: note = DNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
ctcttggttggtattaccaa

SEQ ID NO: 120 - DNA, 20 bp, synthetic construct
  source 1..20: mol_type = other DNA
  misc_feature 1..6: note = RNA
  misc_feature 15..20: note = RNA
  misc_feature 7..14: note = DNA
  modified_base 1..6: mod_base = OTHER; note = 2'-methoxyethyl (2'-MOE) modified nucleoside
  modified_base 15..20: mod_base = OTHER; note = 2'-methoxyethyl (2'-MOE) modified nucleoside
ctcttggttggtattaccaa

SEQ ID NO: 121 - DNA, 20 bp, synthetic construct
  source 1..20: mol_type = other DNA
  misc_feature 1..7: note = RNA
  misc_feature 14..20: note = RNA
  misc_feature 8..13: note = DNA
  modified_base 1..7: mod_base = OTHER; note = 2'-methoxyethyl (2'-MOE) modified nucleoside
  modified_base 14..20: mod_base = OTHER; note = 2'-methoxyethyl (2'-MOE) modified nucleoside
ctcttggttggtattaccaa

SEQ ID NO: 122 - DNA, 18 bp, synthetic construct
  source 1..18: mol_type = other DNA
  misc_feature 1..4: note = RNA
  misc_feature 15..18: note = RNA
  misc_feature 5..14: note = DNA
  modified_base 1..4: mod_base = OTHER; note = 2'-methoxyethyl (2'-MOE) modified nucleoside
  modified_base 15..18: mod_base = OTHER; note = 2'-methoxyethyl (2'-MOE) modified nucleoside
ctcttggttggtattacc

SEQ ID NO: 123 - DNA, 18 bp, synthetic construct
  source 1..18: mol_type = other DNA
  misc_feature 1..5: note = RNA
  misc_feature 15..18: note = RNA
  misc_feature 6..14: note = DNA
  modified_base 1..5: mod_base = OTHER; note = 2'-methoxyethyl (2'-MOE) modified nucleoside
  modified_base 15..18: mod_base = OTHER; note = 2'-methoxyethyl (2'-MOE) modified nucleoside
ctcttggttggtattacc

SEQ ID NO: 124 - DNA, 16 bp, synthetic construct
  source 1..16: mol_type = other DNA
  misc_feature 1..4: note = RNA
  misc_feature 13..16: note = RNA
  misc_feature 5..12: note = DNA
  modified_base 1..4: mod_base = OTHER; note = 2'-methoxyethyl (2'-MOE) modified nucleoside
  modified_base 13..16: mod_base = OTHER; note = 2'-methoxyethyl (2'-MOE) modified nucleoside
cttggttggtattacc

SEQ ID NO: 125 - DNA, 16 bp, synthetic construct
  source 1..16: mol_type = other DNA
  misc_feature 1..5: note = RNA
  misc_feature 12..16: note = RNA
  misc_feature 6..11: note = DNA
  modified_base 1..5: mod_base = OTHER; note = 2'-methoxyethyl (2'-MOE) modified nucleoside
  modified_base 12..16: mod_base = OTHER; note = 2'-methoxyethyl (2'-MOE) modified nucleoside
cttggttggtattacc

SEQ ID NO: 126 - DNA, 17 bp, synthetic construct
  source 1..17: mol_type = other DNA
  misc_feature 1..3: note = RNA
  misc_feature 14..17: note = RNA
  misc_feature 4..13: note = DNA
  modified_base 1..3: mod_base = OTHER; note = locked nucleic acid (LNA) modified nucleoside (cMe; beta-D-methyleneoxy)
  modified_base 14..17: mod_base = OTHER; note = locked nucleic acid (LNA) modified nucleoside (cMe; beta-D-methyleneoxy)
ctcttggttggtattac

SEQ ID NO: 127 - DNA, 17 bp, synthetic construct
  source 1..17: mol_type = other DNA
  misc_feature 1..5: note = RNA
  misc_feature 14..17: note = RNA
  misc_feature 6..13: note = DNA
  modified_base 1..5: mod_base = OTHER; note = locked nucleic acid (LNA) modified nucleoside (cMe; beta-D-methyleneoxy)
  modified_base 14..17: mod_base = OTHER; note = locked nucleic acid (LNA) modified nucleoside (cMe; beta-D-methyleneoxy)
ctcttggttggtattac

SEQ ID NO: 128 - DNA, 16 bp, synthetic construct
  source 1..16: mol_type = other DNA
  modified_base 1..5: mod_base = OTHER; note = locked nucleic acid (LNA) modified nucleoside (cMe; beta-D-methyleneoxy)
  misc_feature 1..5: note = RNA
  misc_feature 13..16: note = RNA
  misc_feature 6..12: note = DNA
  modified_base 13..16: mod_base = OTHER; note = locked nucleic acid (LNA) modified nucleoside (cMe; beta-D-methyleneoxy)
cttggttggtattacc

SEQ ID NO: 129 - DNA, 16 bp, synthetic construct
  source 1..16: mol_type = other DNA
  misc_feature 1..3: note = RNA
  misc_feature 14..16: note = RNA
  misc_feature 4..13: note = DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  modified_base 6: mod_base = OTHER; note = 2'OMe modified deoxynucleoside
cttggttggtattacc

SEQ ID NO: 130 - DNA, 20 bp, synthetic construct
  source 1..20: mol_type = other DNA
  misc_RNA 1..5: note = RNA
  misc_feature 16..20: note = RNA
  misc_feature 6..15: note = DNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
acagcacccaaggtagagac

SEQ ID NO: 131 - DNA, 20 bp, synthetic construct
  source 1..20: mol_type = other DNA
  modified_base 1..6: mod_base = OTHER; note = 2'-methoxyethyl (2'-MOE) modified nucleoside
  misc_feature 1..6: note = RNA
  misc_feature 15..20: note = RNA
  misc_feature 7..14: note = DNA
  modified_base 15..20: mod_base = OTHER; note = 2'-methoxyethyl (2'-MOE) modified nucleoside
acagcacccaaggtagagac

SEQ ID NO: 132 - DNA, 20 bp, synthetic construct
  source 1..20: mol_type = other DNA
  misc_feature 1..7: note = RNA
  misc_feature 14..20: note = RNA
  misc_feature 8..13: note = DNA
  modified_base 1..7: mod_base = OTHER; note = 2'-methoxyethyl (2'-MOE) modified nucleoside
  modified_base 14..20: mod_base = OTHER; note = 2'-methoxyethyl (2'-MOE) modified nucleoside
acagcacccaaggtagagac

SEQ ID NO: 133 - DNA, 18 bp, synthetic construct
  source 1..18: mol_type = other DNA
  misc_feature 1..4: note = RNA
  misc_feature 15..18: note = RNA
  misc_feature 5..14: note = DNA
  modified_base 1..4: mod_base = OTHER; note = 2'-methoxyethyl (2'-MOE) modified nucleoside
  modified_base 15..18: mod_base = OTHER; note = 2'-methoxyethyl (2'-MOE) modified nucleoside
acagcacccaaggtagag

SEQ ID NO: 134 - DNA, 18 bp, synthetic construct
  source 1..18: mol_type = other DNA
  misc_feature 1..5: note = RNA
  misc_feature 14..18: note = RNA
  misc_feature 6..13: note = DNA
  modified_base 1..5: mod_base = OTHER; note = 2'-methoxyethyl (2'-MOE) modified nucleoside
  modified_base 14..18: mod_base = OTHER; note = 2'-methoxyethyl (2'-MOE) modified nucleoside
acagcacccaaggtagag

SEQ ID NO: 135 - DNA, 16 bp, synthetic construct
  source 1..16: mol_type = other DNA
  misc_feature 1..4: note = RNA
  misc_feature 13..16: note = RNA
  misc_feature 5..12: note = DNA
  modified_base 1..4: mod_base = OTHER; note = 2'-methoxyethyl (2'-MOE) modified nucleoside
  modified_base 13..16: mod_base = OTHER; note = 2'-methoxyethyl (2'-MOE) modified nucleoside
agcacccaaggtagag

SEQ ID NO: 136 - DNA, 16 bp, synthetic construct
  source 1..16: mol_type = other DNA
  misc_feature 1..5: note = RNA
  misc_feature 12..16: note = RNA
  misc_feature 6..11: note = DNA
  modified_base 1..5: mod_base = OTHER; note = 2'-methoxyethyl (2'-MOE) modified nucleoside
  modified_base 12..16: mod_base = OTHER; note = 2'-methoxyethyl (2'-MOE) modified nucleoside
agcacccaaggtagag

SEQ ID NO: 137 - DNA, 16 bp, synthetic construct
  source 1..16: mol_type = other DNA
  misc_feature 1..3: note = RNA
  misc_feature 13..16: note = RNA
  misc_feature 4..12: note = DNA
  modified_base 1..3: mod_base = OTHER; note = locked nucleic acid (LNA) modified nucleoside (cMe; beta-D-methyleneoxy)
  modified_base 13..16: mod_base = OTHER; note = locked nucleic acid (LNA) modified nucleoside (cMe; beta-D-methyleneoxy)
acagcacccaaggtag

SEQ ID NO: 138 - DNA, 16 bp, synthetic construct
  source 1..16: mol_type = other DNA
  misc_feature 1..5: note = RNA
  misc_feature 13..16: note = RNA
  misc_feature 6..12: note = DNA
  modified_base 1..5: mod_base = OTHER; note = locked nucleic acid (LNA) modified nucleoside (cMe; beta-D-methyleneoxy)
  modified_base 13..16: mod_base = OTHER; note = locked nucleic acid (LNA) modified nucleoside (cMe; beta-D-methyleneoxy)
acagcacccaaggtag

SEQ ID NO: 139 - DNA, 16 bp, synthetic construct
  source 1..16: mol_type = other DNA
  misc_feature 1..5: note = RNA
  misc_feature 13..16: note = RNA
  misc_feature 6..12: note = DNA
  modified_base 1..5: mod_base = OTHER; note = locked nucleic acid (LNA) modified nucleoside (cMe; beta-D-methyleneoxy)
  modified_base 13..16: mod_base = OTHER; note = locked nucleic acid (LNA) modified nucleoside (cMe; beta-D-methyleneoxy)
agcacccaaggtagag

SEQ ID NO: 140 - DNA, 16 bp, synthetic construct
  source 1..16: mol_type = other DNA
  misc_feature 1..3: note = RNA
  misc_feature 14..16: note = RNA
  misc_feature 4..13: note = DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  modified_base 6: mod_base = OTHER; note = 2'OMe modified deoxynucleoside
agcacccaaggtagag

SEQ ID NO: 141 - DNA, 20 bp, synthetic construct
  source 1..20: mol_type = other DNA
  misc_feature 1..5: note = RNA
  modified_base 1..5: mod_base = OTHER; note = 2'-methoxyethyl (2'-MOE) modified nucleoside
  misc_feature 16..20: note = RNA
  modified_base 16..20: mod_base = OTHER; note = 2'-methoxyethyl (2'-MOE) modified nucleoside
  misc_feature 6..15: note = DNA
tccactttgtcccacatata

SEQ ID NO: 142 - DNA, 20 bp, synthetic construct
  source 1..20: mol_type = other DNA
  misc_feature 1..6: note = RNA
  misc_feature 15..20: note = RNA
  misc_feature 7..14: note = DNA
  modified_base 1..6: mod_base = OTHER; note = 2'-methoxyethyl (2'-MOE) modified nucleoside
  modified_base 15..20: mod_base = OTHER; note = 2'-methoxyethyl (2'-MOE) modified nucleoside
tccactttgtcccacatata

SEQ ID NO: 143 - DNA, 20 bp, synthetic construct
  source 1..20: mol_type = other DNA
  misc_feature 1..7: note = RNA
  misc_feature 14..20: note = RNA
  misc_feature 8..13: note = DNA
  modified_base 1..7: mod_base = OTHER; note = 2'-methoxyethyl (2'-MOE) modified nucleoside
  modified_base 14..20: mod_base = OTHER; note = 2'-methoxyethyl (2'-MOE) modified nucleoside
tccactttgtcccacatata

SEQ ID NO: 144 - DNA, 18 bp, synthetic construct
  source 1..18: mol_type = other DNA
  modified_base 1..4: mod_base = OTHER; note = 2'-methoxyethyl (2'-MOE) modified nucleoside
  misc_feature 1..4: note = RNA
  misc_feature 15..18: note = RNA
  misc_feature 5..14: note = DNA
  modified_base 15..18: mod_base = OTHER; note = 2'-methoxyethyl (2'-MOE) modified nucleoside
tccactttgtcccacata

SEQ ID NO: 145 - DNA, 18 bp, synthetic construct
  source 1..18: mol_type = other DNA
  misc_feature 1..5: note = RNA
  misc_RNA 14..18: note = RNA
  misc_feature 6..13: note = DNA
  modified_base 1..5: mod_base = OTHER; note = 2'-methoxyethyl (2'-MOE) modified nucleoside
  modified_base 14..18: mod_base = OTHER; note = 2'-methoxyethyl (2'-MOE) modified nucleoside
tccactttgtcccacata

SEQ ID NO: 146 - DNA, 16 bp, synthetic construct
  source 1..16: mol_type = other DNA
  modified_base 1..4: mod_base = OTHER; note = 2'-methoxyethyl (2'-MOE) modified nucleoside
  modified_base 13..16: mod_base = OTHER; note = 2'-methoxyethyl (2'-MOE) modified nucleoside
  misc_feature 1..4: note = RNA
  misc_feature 13..16: note = RNA
  misc_feature 5..12: note = DNA
cactttgtcccacata

SEQ ID NO: 147 - DNA, 16 bp, synthetic construct
  source 1..16: mol_type = other DNA
  misc_feature 1..5: note = RNA
  misc_RNA 12..16: note = RNA
  misc_feature 6..11: note = DNA
  modified_base 1..5: mod_base = OTHER; note = 2'-methoxyethyl (2'-MOE) modified nucleoside
  modified_base 12..16: mod_base = OTHER; note = 2'-methoxyethyl (2'-MOE) modified nucleoside
cactttgtcccacata

SEQ ID NO: 148 - DNA, 17 bp, synthetic construct
  source 1..17: mol_type = other DNA
  misc_feature 1..3: note = RNA
  misc_feature 14..17: note = RNA
  misc_feature 4..13: note = DNA
  modified_base 14..17: mod_base = OTHER; note = locked nucleic acid (LNA) modified nucleoside (cMe; beta-D-methyleneoxy)
  modified_base 1..3: mod_base = OTHER; note = locked nucleic acid (LNA) modified nucleoside (cMe; beta-D-methyleneoxy)
tccactttgtcccacat

SEQ ID NO: 149 - DNA, 17 bp, synthetic construct
  source 1..17: mol_type = other DNA
  misc_feature 1..5: note = RNA
  misc_feature 14..17: note = RNA
  misc_feature 6..13: note = DNA
  modified_base 1..5: mod_base = OTHER; note = locked nucleic acid (LNA) modified nucleoside (cMe; beta-D-methyleneoxy)
  modified_base 14..17: mod_base = OTHER; note = locked nucleic acid (LNA) modified nucleoside (cMe; beta-D-methyleneoxy)
tccactttgtcccacat

SEQ ID NO: 150 - DNA, 16 bp, synthetic construct
  source 1..16: mol_type = other DNA
  misc_feature 1..5: note = RNA
  misc_feature 13..16: note = RNA
  misc_difference 6..12: note = DNA
  modified_base 1..5: mod_base = OTHER; note = locked nucleic acid (LNA) modified nucleoside (cMe; beta-D-methyleneoxy)
  modified_base 13..16: mod_base = OTHER; note = locked nucleic acid (LNA) modified nucleoside (cMe; beta-D-methyleneoxy)
cactttgtcccacata

SEQ ID NO: 151 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 14..16: note = RNA
  misc_feature 4..13: note = DNA
  modified_base 14..16: mod_base = OTHER; note = locked nucleic acid (LNA) modified nucleoside (cMe; beta-D-methyleneoxy)
  modified_base 6: mod_base = OTHER; note = 2'OMe modified deoxynucleoside
cactttgtcccacata

SEQ ID NO: 152 - DNA, 20 bp, synthetic construct
  source 1..20: mol_type = other DNA
  misc_feature 1..5: note = RNA
  misc_feature 16..20: note = RNA
  misc_feature 6..15: note = DNA
  modified_base 16..20: mod_base = OTHER; note = 2'-methoxyethyl (2'-MOE) modified nucleoside
  modified_base 1..5: mod_base = OTHER; note = 2'-methoxyethyl (2'-MOE) modified nucleoside
gatagagtctcagatggata

SEQ ID NO: 153 - DNA, 20 bp, synthetic construct
  source 1..20: mol_type = other DNA
  misc_feature 1..6: note = RNA
  misc_feature 16..20: note = RNA
  misc_feature 7..15: note = DNA
  modified_base 1..6: mod_base = OTHER; note = 2'-methoxyethyl (2'-MOE) modified nucleoside
  modified_base 16..20: mod_base = OTHER; note = 2'-methoxyethyl (2'-MOE) modified nucleoside
gatagagtctcagatggata

SEQ ID NO: 154 - DNA, 20 bp, synthetic construct
  source 1..20: mol_type = other DNA
  misc_feature 1..7: note = RNA
  misc_feature 14..20: note = RNA
  misc_feature 8..13: note = DNA
  modified_base 1..7: mod_base = OTHER; note = 2'-methoxyethyl (2'-MOE) modified nucleoside
  modified_base 14..20: mod_base = OTHER; note = 2'-methoxyethyl (2'-MOE) modified nucleoside
gatagagtctcagatggata

SEQ ID NO: 155 - DNA, 18 bp, synthetic construct
  source 1..18: mol_type = other DNA
  misc_feature 1..4: note = RNA
  misc_feature 15..18: note = RNA
  misc_feature 5..14: note = DNA
  modified_base 1..4: mod_base = OTHER; note = 2'-methoxyethyl (2'-MOE) modified nucleoside
  modified_base 15..18: mod_base = OTHER; note = 2'-methoxyethyl (2'-MOE) modified nucleoside
gatagagtctcagatgga

SEQ ID NO: 156 - DNA, 18 bp, synthetic construct
  source 1..18: mol_type = other DNA
  misc_feature 1..5: note = RNA
  misc_feature 14..18: note = RNA
  misc_feature 6..13: note = DNA
  modified_base 1..5: mod_base = OTHER; note = 2'-methoxyethyl (2'-MOE) modified nucleoside
  modified_base 14..18: mod_base = OTHER; note = 2'-methoxyethyl (2'-MOE) modified nucleoside
gatagagtctcagatgga

SEQ ID NO: 157 - DNA, 16 bp, synthetic construct
  source 1..16: mol_type = other DNA
  misc_feature 1..4: note = RNA
  misc_feature 13..16: note = RNA
  misc_feature 5..12: note = DNA
  modified_base 1..4: mod_base = OTHER; note = 2'-methoxyethyl (2'-MOE) modified nucleoside
  modified_base 13..16: mod_base = OTHER; note = 2'-methoxyethyl (2'-MOE) modified nucleoside
tagagtctcagatgga

SEQ ID NO: 158 - DNA, 16 bp, synthetic construct
  source 1..16: mol_type = other DNA
  modified_base 1..5: mod_base = OTHER; note = 2'-methoxyethyl (2'-MOE) modified nucleoside
  modified_base 12..16: mod_base = OTHER; note = 2'-methoxyethyl (2'-MOE) modified nucleoside
  misc_feature 1..5: note = RNA
  misc_feature 12..16: note = RNA
  misc_feature 6..11: note = DNA
tagagtctcagatgga

SEQ ID NO: 159 - DNA, 17 bp, synthetic construct
  source 1..17: mol_type = other DNA
  misc_feature 1..3: note = RNA
  misc_feature 14..17: note = RNA
  misc_feature 4..13: note = DNA
  modified_base 1..3: mod_base = OTHER; note = locked nucleic acid (LNA) modified nucleoside (cMe; beta-D-methyleneoxy)
  modified_base 14..17: mod_base = OTHER; note = locked nucleic acid (LNA) modified nucleoside (cMe; beta-D-methyleneoxy)
gatagagtctcagatgg

SEQ ID NO: 160 - DNA, 17 bp, synthetic construct
  source 1..17: mol_type = other DNA
  misc_feature 1..5: note = RNA
  misc_feature 14..17: note = RNA
  misc_feature 6..13: note = DNA
  modified_base 1..5: mod_base = OTHER; note = locked nucleic acid (LNA) modified nucleoside (cMe; beta-D-methyleneoxy)
  modified_base 14..17: mod_base = OTHER; note = locked nucleic acid (LNA) modified nucleoside (cMe; beta-D-methyleneoxy)
gatagagtctcagatgg

SEQ ID NO: 161 - DNA, 16 bp, synthetic construct
  source 1..16: mol_type = other DNA
  misc_feature 1..5: note = RNA
  misc_feature 13..16: note = RNA
  misc_feature 6..12: note = DNA
  modified_base 1..5: mod_base = OTHER; note = locked nucleic acid (LNA) modified nucleoside (cMe; beta-D-methyleneoxy)
  modified_base 13..16: mod_base = OTHER; note = locked nucleic acid (LNA) modified nucleoside (cMe; beta-D-methyleneoxy)
tagagtctcagatgga

SEQ ID NO: 162 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 6: mod_base = OTHER; note = 2'OMe modified deoxynucleoside
  misc_feature 1..3: note = RNA
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
tagagtctcagatgga

SEQ ID NO: 163 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
tcataacctggactct

SEQ ID NO: 164 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
tcccctgggcagtacc

SEQ ID NO: 165 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
tacaaatggagggctc

SEQ ID NO: 166 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
tctacaaatggagggc

SEQ ID NO: 167 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
actctcattcattcta

SEQ ID NO: 168 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
tggactctcattcatt

SEQ ID NO: 169 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
ataacctggactctca

SEQ ID NO: 170 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
gtgttcctacactcca

SEQ ID NO: 171 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
ggtgttcctacactcc

SEQ ID NO: 172 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
agggtgttcctacact

SEQ ID NO: 173 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
caaaggagggtgttcc

SEQ ID NO: 174 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
gcagacctgtcaaaga

SEQ ID NO: 175 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
gatgcagacctgtcaa

SEQ ID NO: 176 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
tgatgcagacctgtca

SEQ ID NO: 177 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
agtgttatgatgcaga

SEQ ID NO: 178 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
gggggccgagatcgtg

SEQ ID NO: 179 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
ctagggtaacagcacc

SEQ ID NO: 180 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
tagggtaacagcacct

SEQ ID NO: 181 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
gctagggtaacagcac

SEQ ID NO: 182 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
cagatggatattctta

SEQ ID NO: 183 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
tggctagggtaacagc

SEQ ID NO: 184 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
ttggctagggtaacag

SEQ ID NO: 185 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
attggctagggtaaca

SEQ ID NO: 186 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
attaccaagagataga

SEQ ID NO: 187 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
gatctggttcatccat

SEQ ID NO: 188 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
ttgatctggttcatcc

SEQ ID NO: 189 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
attgatctggttcatc

SEQ ID NO: 190 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
ctcatctggcattgat

SEQ ID NO: 191 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
gagttcaccaaaaaat

SEQ ID NO: 192 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
ctcttggttggtatta

SEQ ID NO: 193 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
actcttggttggtatt

SEQ ID NO: 194 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
aactgataactcttgg

SEQ ID NO: 195 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
aaactgataactcttg

SEQ ID NO: 196 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
gagaaactgataactc

SEQ ID NO: 197 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNa
  misc_feature 4..13: note = DNA
agagaaactgataact

SEQ ID NO: 198 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
cggttaaagagaaact

SEQ ID NO: 199 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
tcggttaaagagaaac

SEQ ID NO: 200 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
ctcggttaaagagaaa

SEQ ID NO: 201 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
tgtctcggttaaagag

SEQ ID NO: 202 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
gtgtctcggttaaaga

SEQ ID NO: 203 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
ggtgtctcggttaaag

SEQ ID NO: 204 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
tggtgtctcggttaaa

SEQ ID NO: 205 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
ctggtgtctcggttaa

SEQ ID NO: 206 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
ttgctggtgtctcggt

SEQ ID NO: 207 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
taccctggagcaggca

SEQ ID NO: 208 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
gtaccctggagcaggc

SEQ ID NO: 209 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
tccgtcaggagcctga

SEQ ID NO: 210 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
gtcaggagcctgaacc

SEQ ID NO: 211 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
actccgtcaggagcct

SEQ ID NO: 212 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  rRNA 1..3: note = RNA
  misc_feature 14..16: note = RNA
  misc_feature 4..13: note = DNA
aactccgtcaggagcc

SEQ ID NO: 213 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
caactccgtcaggagc

SEQ ID NO: 214 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
cgtcaggagcctgaac

SEQ ID NO: 215 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
ccaactccgtcaggag

SEQ ID NO: 216 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
gccccaactccgtcag

SEQ ID NO: 217 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
tgccccaactccgtca

SEQ ID NO: 218 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
ttgccccaactccgtc

SEQ ID NO: 219 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
gttgccccaactccgt

SEQ ID NO: 220 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
ggttgccccaactccg

SEQ ID NO: 221 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
gggttgccccaactcc

SEQ ID NO: 222 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
tccagggttgccccaa

SEQ ID NO: 223 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
gtgagcttatctccag

SEQ ID NO: 224 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
gtgtgagcttatctcc

SEQ ID NO: 225 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
tgtgtgagcttatctc

SEQ ID NO: 226 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
ttgtgtgagcttatct

SEQ ID NO: 227 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
gttgtgtgagcttatc

SEQ ID NO: 228 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
ggttgtgtgagcttat

SEQ ID NO: 229 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
ttgcagggttgtgtga

SEQ ID NO: 230 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
cttgcagggttgtgtg

SEQ ID NO: 231 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
ggtcttgcagggttgt

SEQ ID NO: 232 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
gcacaacgattgtcag

SEQ ID NO: 233 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA; note = RNA
  misc_feature 4..13: note = DNA
tgcacaacgattgtca

SEQ ID NO: 234 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
ctgcacaacgattgtc

SEQ ID NO: 235 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
gtgggaagattcctaa

SEQ ID NO: 236 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
tgtcccacatatacta

SEQ ID NO: 237 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
tatccatctgtaagtg

SEQ ID NO: 238 - DNA, 16 bp, synthetic construct
  source 1..16: mol_type = other DNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  misc_feature 1..3: note = RNA
  misc_feature 14..16: note = RNA
  misc_feature 4..13: note = DNA
aatgtggacatcaact

SEQ ID NO: 239 - DNA, 20 bp, synthetic construct
  source 1..20: mol_type = other DNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
  misc_feature 1..5: note = RNA
  misc_feature 6..15: note = DNA
  misc_feature 16..20: note = RNA
agctcctgaggcagagaaca

SEQ ID NO: 240 - DNA, 20 bp, synthetic construct
  source 1..20: mol_type = other DNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
  misc_feature 16..20: note = RNA
  misc_feature 1..5: note = RNA
  misc_feature 6..15: note = DNA
agctcccgaggcagagaaca

(19) United States
(12) Patent Application Publication
(10) Pub. No.: US 2025/0115909 A1
(43) Pub. Date: Apr. 10, 2025

(54) COMPOUNDS FOR INHIBITING AMANZI

(71) Applicant: Lemba BV, Nijmegen (NL)

(72) Inventors: Ezio Fok, Nijmegen (NL); Stephanie Berry, Scarsdale, NY (US); Makiri Kawasaki, Tokyo (JP)

(21) Appl. No.: 18/906,676

(22) Filed: Oct. 4, 2024

Related U.S. Application Data
(60) Provisional application No. 63/542,512, filed on Oct. 4, 2023.

(51) Int. Cl. C12N 15/113

(52) U.S. Cl. CPC C12N 15/113; C12N 2310/113; C12N 2310/315; C12N 2310/322; C12N 2310/3231; C12N 2310/341

(57) ABSTRACT

The inventions relate to an ASO (antisense oligonucleotide) compound that can inhibit AMANZI transcriptional activity. More specifically, the disclosed ASO comprises a gapmer type of ASO comprising from about 14 to about 25 nucleotide bases; (a) a 3′ wing region (3′ to 5′) having from 3 to 7 chemically modified RNA bases; (b) a gap region having from at least 8 DNA bases to no more than 19 DNA bases; and (c) a 5′ wing region (3′ to 5′) having from 3 to 7 chemically modified RNA bases. Preferably, the gapmer is substantially complementary to a region on AMANZI (SEQ ID NO. 1). More preferably, the gapmer is substantially complementary to a 14-25 base region on AMANZI.

### PRIOR RELATED APPLICATIONS

This application claims priority to U.S. Provisional Application Ser. No. 63/542,512, filed Oct. 4, 2023, the contents of which are herein incorporated in their entirety.

### SEQUENCE STATEMENT

The instant application contains a Sequence Listing, which has been submitted electronically and is hereby incorporated by reference in its entirety. Said file is named J1742-00202_SL SEQ.xml and is about 747 kb in size.

### FIELD

The inventions relate to compounds and methods for modulating AMANZI transcriptional activity. In particular are provided gapmer type of ASO (antisense oligonucleotide) compounds that can modulate AMANZI transcriptional activity relating to inflammation and activation of the immune system, comprising (a) from about 14 to about 25 nucleotide bases; (b) a 3′ wing region (3′ to 5′) having from 3 to 7 chemically modified RNA bases; (c) a gap region having from at least 8 DNA bases to no more than 19 DNA bases; and (d) a 5′ wing region (3′ to 5′) having from 3 to 7 chemically modified RNA bases; wherein the gapmer is substantially complementary to a 14-25 base region on AMANZI (SEQ ID NO. 1). In particular, the gapmer nucleotide bases can each be linked by phosphorothiolate (P═S) internucleotide bonds throughout the gapmer; and the modified nucleotide base modifications can be selected from the group consisting of 2′-methoxyethyl (MOE) nucleotides, locked nucleic acid nucleotides (LNA), and combinations thereof.

### BACKGROUND

The following includes information that may be useful in understanding the present invention. It is not an admission that any of the information, publications or documents specifically or implicitly referenced herein is prior art, or important, to the inventions described and claimed herein. All publications, patents, related applications, and other written or electronic materials mentioned or identified herein are hereby incorporated herein by reference in their entirety. The information incorporated is as much a part of the application as filed as if all of the text and all other content was repeated in the application and should be treated as part of the text and content of the application as filed.

Long non-coding RNAs (lncRNAs) are non-coding transcripts of more typically than 200 nucleotides (200 nt). lncRNAs can serve as a scaffold for the assembly of chromatin remodeling complexes to robustly influence gene transcription at specific loci. Mattick, J. S., et al. Nat Rev Mol Cell Biol 24, 430-447 (2023)). Long non-coding transcripts are found in many species. DNA (cDNA) sequencing projects such as FANTOM have identified the complexity of these transcripts in humans (Carninci P, et al. (September 2005), Science. 309 (5740): 1559-1563).

Inflammation is characterized by a biphasic cycle consisting initially of a proinflammatory phase that is subsequently resolved by anti-inflammatory processes. Interleukin-1β (IL-1β) is a master regulator of proinflammation and is encoded within the same topologically associating domain (TAD) as IL-37, which is an anti-inflammatory cytokine that opposes the function of IL-1β.

### SUMMARY

The inventions described and claimed herein have many attributes and embodiments including, but not limited to, those set forth or described or referenced in this Brief Summary. It is not intended to be all-inclusive and the inventions described and claimed herein are not limited to or by the features or embodiments identified in this introduction, which is included for purposes of illustration only and not restriction.

A MAster Non coding RNA antagoniZing Inflammation (AMANZI) is a lncRNA encoded within the promoter of IL-1B. AMANZI is co-transcribed from the opposite strand and opposite direction from IL-1B and mediates a temporally delayed long-range chromatin looping interaction with the promoter of IL-37, which is otherwise spatially insulated from the active IL-1β locus. AMANZI is a suppressor of IL-1β.

The present disclosure provides an ASO (antisense oligonucleotide) compound can lead to over-expression of IL-1B. The present disclosure provides a gapmer type of ASO (antisense oligonucleotide) that can inhibit AMANZI transcriptional activity. More specifically, the disclosed ASO comprises a Gapmer type of ASO comprising from about 14 to about 25 nucleotide bases; a 3′ wing region (3′ to 5′) having from 3 to 7 chemically modified RNA bases; a gap region having from at least 8 DNA bases to no more than 19 DNA bases; and a 5′ wing region (3′ to 5′) having from 3 to 7 chemically modified RNA bases. Preferably, the gapmer is substantially complementary to a 14-25 base region on AMANZI (SEQ ID NO. 1). Preferably, the gapmer nucleotide bases are each linked by phosphorothiolate (P═S) internucleotide bonds throughout the gapmer; and wherein the modified nucleotide base modifications are selected from the group consisting of 2′-methoxyethyl (MOE) nucleotides, locked nucleic acid nucleotides (LNA), and combinations thereof.

The present disclosure provides a gapmer type of ASO (antisense oligonucleotide) that can inhibit AMANZI transcriptional activity, comprising (a) from about 14 to about 25 nucleotide bases; (b) a 3′ wing region (3′ to 5′) having from 3 to 7 chemically modified RNA bases; (c) a gap region having from at least 8 DNA bases to no more than 19 DNA bases; and (d) a 5′ wing region (3′ to 5′) having from 3 to 7 chemically modified RNA bases; wherein the gapmer is substantially complementary to substantially complementary to Region A of AMANZI (SEQ ID NO. 1 bases 10 to 93). Preferably, the gap region comprises a 10-nucleotide DNA sequence from nucleotide 5 to nucleotide 15 from any of SEQ ID NOs. 2-105 or an 8 mer fragment thereof. Preferably, the gapmer nucleotides are each linked by phosphorothiolate (P═S) internucleotide bonds throughout the gapmer; and wherein the modified nucleotide base modifications are selected from the group consisting of 2′-methoxyethyl (MOE) nucleotides, locked nucleic acid nucleotides (LNA), and combinations thereof. Preferably, Region A of AMANZI is SEQ ID NO. 1 base 12 to base 27. More preferably, the gapmer type of ASO is selected from the group consisting of SEQ ID NO. 41, SEQ ID NO. 42, SEQ ID NO. 43, SEQ ID NO. 44, SEQ ID NO. 45, SEQ ID NO. 46, and combinations thereof. Most preferably the gapmer type of ASO is SEQ ID NO. 42.

The present disclosure provides a gapmer type of ASO (antisense oligonucleotide) that can inhibit AMANZI transcriptional activity, comprising (a) from about 14 to about 25 nucleotide bases; (b) a 3′ wing region (3′ to 5′) having from 3 to 7 chemically modified RNA bases; (c) a gap region having from at least 8 DNA bases to no more than 19 DNA bases; and (d) a 5′ wing region (3′ to 5′) having from 3 to 7 chemically modified RNA bases; wherein the gapmer is substantially complementary to substantially complementary Region B of AMANZI (SEQ ID NO. 1 bases 194 to 253). Preferably, the gap region comprises a 10-nucleotide DNA sequence from nucleotide 5 to nucleotide 15 from any of SEQ ID NO. 78, SEQ ID NO. 81, SEQ ID NO. 82, SEQ ID NO. 83, SEQ ID NO. 84, and combinations thereof, or an 8 mer fragment thereof. Preferably, the gapmer nucleotides are each linked by phosphorothiolate (P═S) internucleotide bonds throughout the gapmer; and wherein the modified nucleotide base modifications are selected from the group consisting of 2′-methoxyethyl (MOE) nucleotides, locked nucleic acid nucleotides (LNA), and combinations thereof. More preferably, the gapmer type of ASO is selected from the group consisting of SEQ ID NO. 78, SEQ ID NO. 81, SEQ ID NO. 82, SEQ ID NO. 83, SEQ ID NO. 84, and combinations thereof. Most preferably the gapmer type of ASO is SEQ ID NO. 84.

The present disclosure a gapmer type of ASO (antisense oligonucleotide) that can inhibit AMANZI transcriptional activity, wherein the gapmer comprises (a) from about 14 to about 25 nucleotide bases; (b) a 3′ wing region (3′ to 5′) having from 3 to 7 chemically modified RNA bases; (c) a gap region having from at least 8 DNA bases to no more than 19 DNA bases; and (d) a 5′ wing region (3′ to 5′) having from 3 to 7 chemically modified RNA bases; wherein the gapmer is substantially complementary to substantially complementary Region C of AMANZI (SEQ ID NO. 1 bases 519 to 568). Preferably, the gap region comprises a 10-nucleotide DNA sequence from nucleotide 5 to nucleotide 15 from any of SEQ ID NO. 63, SEQ ID NO. 64, SEQ ID NO. 66, SEQ ID NO. 67, SEQ ID NO. 69, SEQ ID NO. 101, SEQ ID NO. 102, SEQ ID NO. 103, SEQ ID NO. 104, and combinations thereof, or an 8 mer fragment thereof. Preferably, the gapmer nucleotides are each linked by phosphorothiolate (P═S) internucleotide bonds throughout the gapmer; and wherein the modified nucleotide base modifications are selected from the group consisting of 2′-methoxyethyl (MOE) nucleotides, locked nucleic acid nucleotides (LNA), and combinations thereof. More preferably, the gapmer type of ASO is selected from the group consisting of SEQ ID NO. 63, SEQ ID NO. 64, SEQ ID NO. 66, SEQ ID NO. 67, SEQ ID NO. 69, SEQ ID NO. 101, SEQ ID NO. 102, SEQ ID NO. 103, SEQ ID NO. 104, and combinations thereof. Most preferably the gapmer type of ASO is SEQ ID NO. 101.

The present disclosure provides a gapmer type of ASO (antisense oligonucleotide) that can inhibit AMANZI transcriptional activity, wherein the gapmer comprises (a) from about 14 to about 25 nucleotide bases; (b) a 3′ wing region (3′ to 5′) having from 3 to 7 chemically modified RNA bases; (c) a gap region having from at least 8 DNA bases to no more than 19 DNA bases; and (d) a 5′ wing region (3′ to 5′) having from 3 to 7 chemically modified RNA bases; wherein the gapmer is substantially complementary to substantially complementary to Region D of AMANZI (SEQ ID NO. 1 bases 377 to 404). Preferably, the gap region comprises a 10-nucleotide DNA sequence from nucleotide 5 to nucleotide 15 from any of SEQ ID NOs. 228-234, and combinations thereof, or an 8-mer fragment thereof. Preferably, the gapmer nucleotides arc each linked by phosphorothiolate (P═S) internucleotide bonds throughout the gapmer; and wherein the modified nucleotide base modifications are selected from the group consisting of 2′-methoxyethyl (MOE) nucleotides, locked nucleic acid nucleotides (LNA), and combinations thereof.

The present disclosure provides a gapmer type of ASO (antisense oligonucleotide) that can inhibit AMANZI transcriptional activity, wherein the gapmer comprises (a) from about 14 to about 25 nucleotide bases; (b) a 3′ wing region (3′ to 5′) having from 3 to 7 chemically modified RNA bases; (c) a gap region having from at least 8 DNA bases to no more than 19 DNA bases; and (d) a 5′ wing region (3′ to 5′) having from 3 to 7 chemically modified RNA bases; wherein the gapmer is substantially complementary to substantially complementary Region E of AMANZI (SEQ ID NO. 1 bases 574 to 615). Preferably, the gap region comprises a 10-nucleotide DNA sequence from nucleotide 5 to nucleotide 15 from any of SEQ ID NOs. 206-215, and combinations thereof, or an 8 mer fragment thereof. Preferably, the gapmer nucleotides are each linked by phosphorothiolate (P═S) internucleotide bonds throughout the gapmer; and wherein the modified nucleotide base modifications are selected from the group consisting of 2′-methoxyethyl (MOE) nucleotides, locked nucleic acid nucleotides (LNA), and combinations thereof.

The present disclosure provides a pharmaceutical composition comprising a gapmer type of ASO (antisense oligonucleotide) that can inhibit AMANZI transcriptional activity and pharmaceutical excipients, wherein the gapmer comprises (a) from about 14 to about 25 nucleotide bases; (b) a 3′ wing region (3′ to 5′) having from 3 to 7 chemically modified RNA bases; (c) a gap region having from at least 8 DNA bases to no more than 19 DNA bases; and (d) a 5′ wing region (3′ to 5′) having from 3 to 7 chemically modified RNA bases; wherein the gapmer is substantially complementary to substantially complementary Region D of AMANZI (SEQ ID NO. 1 bases 377 to 404). Preferably, the gap region comprises a 10-nucleotide DNA sequence from nucleotide 5 to nucleotide 15 from any of SEQ ID Nos. 228-234, and combinations thereof, or an 8-mer fragment thereof. Preferably, the gapmer nucleotides are each linked by phosphorothiolate (P═S) internucleotide bonds throughout the gapmer; and wherein the modified nucleotide base modifications are selected from the group consisting of 2′-methoxyethyl (MOE) nucleotides, locked nucleic acid nucleotides (LNA), and combinations thereof.

The present disclosure provides a pharmaceutical composition comprising a gapmer type of ASO (antisense oligonucleotide) that can inhibit AMANZI transcriptional activity and pharmaceutical excipients, wherein the gapmer comprises (a) from about 14 to about 25 nucleotide bases; (b) a 3′ wing region (3′ to 5′) having from 3 to 7 chemically modified RNA bases; (c) a gap region having from at least 8 DNA bases to no more than 19 DNA bases; and (d) a 5′ wing region (3′ to 5′) having from 3 to 7 chemically modified RNA bases; wherein the gapmer is substantially complementary to substantially complementary Region E of AMANZI (SEQ ID NO. 1 bases 574 to 615). Preferably, the gap region comprises a 10-nucleotide DNA sequence from nucleotide 5 to nucleotide 15 from any of SEQ ID NOs. 206-215, and combinations thereof, or an 8 mer fragment thereof. Preferably, the gapmer nucleotides are each linked by phosphorothiolate (P═S) internucleotide bonds throughout the gapmer; and wherein the modified nucleotide base modifications are selected from the group consisting of 2′-methoxyethyl (MOE) nucleotides, locked nucleic acid nucleotides (LNA), and combinations thereof.

The gapmer nucleotide bases can each be independently linked by internucleotide bonds throughout the gapmer wherein at least one of said internucleotide bond is a phosphorothiolate (P═S) internucleotide bonds; and wherein nucleotide bases include at least one modified nucleotide base modification selected from the group consisting of 2′-methoxyethyl (MOE) nucleotides, locked nucleic acid nucleotides (LNA), and combinations thereof. In some aspects, at least one modified internucleoside linkage is a phosphorothioate linkage. In some aspects, all of the internucleotide bonds are phosphorothioate bonds throughout the gapmer.

### DETAILED DISCLOSURE

The present disclosure is based on investigations that inhibitors of AMANZI transcriptional activity can lead to over-expression of IL-1ß (beta).

The inventions relate to AMANZI transcriptional modulators and compositions comprising thereof for the modulation of AMANZI transcription. In some embodiments, the AMANZI transcriptional modulators are AMANZI transcriptional inhibitors.

### Target AMANZI

(FANTOM CAT no: FTMT20800006495.1)
Human AMANZI sequence:
(SEQ ID NO. 1)
CTGACAATCGTTGTGCAGTTGATGTCCACATTAAAATACTGGATTTTCC
CACGTTAGAAGAATGTTTAAATTTAGTATATGTGGGACAAAGTGGAAGA
CACACAGATTTATACATGCACATACTTTTCTTCATTCACTTCTTTGTAC
TTAAGTTTAGGAATCTTCCCACTTACAGATGGATAAATGGGTACAATGA
AGGGCCAATAGCCCTCCCTGTCTGTATTGAGGGTGTGGGTCTCTACCTT
GGGTGCTGTTCTCTGCCTCAGGAGCTCTCTGTCAATTGCAGGAGCCTCT
GAGGAGAAAATTGACCTTTCTTGGCTGGGGCAGAGAACATACGGTATGC
AGGGTTCAGGCTCCTGACGGAGTTGGGGCAACCCTGGAGATAAGCTCAC
ACAACCCTGCAAGACCAGGTGCTGTTACCCTAGCCAATCTCATGGATGA
ACCAGATCAATGCCAGATGAGCTCTGCCTAAAATGATTTTTTGGTGAAC
TCTGAAAAGTGGAATATTGTTTCTGTAAGAATATCCATCTGAGACTCTA
TCTCTTGGTAATACCAACCAAGAGTTATCAGTTTCTCTTTAACCGAGAC
ACCAGCAAAGTGCCTGCTCCAGGGTACTGCCCAGGGGAGCCCTCCATTT
GTAGAATGAATGAGAGTCCAGGTTATGAACAGTGCCTGGAGTGTAGGAA
CACCCTCCTTTGCCTCTTTGACAGGTCTGCATCATAACACTTTTTTTTT
TTTTTTGAGACAGAGTCTCACTCTGTCGCCCAGGCTGGAGTGCAGTGGC
ACGATCTCGGCCCCCTGCAAGTTCCGCCTCCCGGGTTCACACCATTCTC
CTGCCTCAGCCTCCCCAGCAGCTGGGACTACAGGCACCTGCCGCCACGC
CCGGCTAATTTTTTGTATTTTTAGTAGAGACAGGGTTTCACCATGTTAG
CCAGGATGGTCTCGATCTCCTGACCTTGTGATCTGCCCGCCTCGGCCTC
CCAAAGTGTTGGGATTACAGGCGTGAGCCACCGTGTCCAGCCTGTAACA
CTTCTTATAGCACTGAGTTGAAACCTTGCTCCTCCTGGTTCCTCCAGGA
AACTGAAATCTTTTTGAGCCAAGTCTAGCACAGTGCCTGGCATGTACAT
TCAGGTGGTAGAGTTTGCTGCTTGAATGGGTGAATGGGAATTTGACAGC
ATTTTTATTCAAATTAGTATGTGCCAGGTATCGTGCTCGCTCTGCATTA
TCCAAGGGAGTGAGCCTCTGTGCAAGTATTTGAGACACGAGGGAAATAG
GTTCTACTGTGGGAAAAAGAGCATTTCATGGACTTGCTCTCCAAGCAGC
CTTCTGATTTTTAATTTGGCTCCCAGTATCTTGATATCAGGAGTCAGTC
ACAAGAACTCCATCTTTAGTAAGTTATATTTTCCACAGGAAATCTAAAA
GCTGTTCAACATGTTAGTTTCCTGTGAATTTGATAAGCCATAATCCATT
CCTAACACTGAGCCCTCCTGAAATTTGGTGTCTGGTCCTGCAGATAGCT
AAAAGCCCTGTCTGGGTGGCCTAGGGGACTCCTCTGTTTTGCCTCCACA
G

### Definitions

The following terms have the following meanings:

“2′-substituted nucleoside” means a nucleoside comprising a 2′-substituted sugar moiety. “2′-substituted” in reference to a sugar moiety means a sugar moiety comprising at least one 2′-substituent group other than H or OH.

“2′-deoxynucleoside” means a nucleoside comprising 2′-H furanosyl sugar moiety, as found naturally occurring in deoxyribonucleosides (DNA). A 2′-deoxynucleoside may comprise a modified nucleobase or may comprise an RNA nucleobase (e.g., uracil).

“2′-O-methoxyethyl” (also 2′-MOE and 2′-O(CH₂)₂—OCH₃) refers to an O-methoxy-ethyl modification of the 2′ position of a furosyl ring. A 2′-O-methoxyethyl modified sugar is a modified sugar.

“2′-O-methoxyethyl nucleotide” means a nucleotide comprising a 2′-O-methoxyethyl modified sugar moiety.

“5-methyl cytosine” means a cytosine modified with a methyl group attached to a 5 position. A 5-methyl cytosine is a modified nucleobase.

“About” means plus or minus 7% of the provided value.

“Active target region” or “target region” means a region to which one or more active antisense compounds is targeted. “Active antisense compounds” means antisense compounds that reduce target gene transcription or resulting protein levels.

“Adjuvant” is defined as any molecule to enhance an antigen-specific adaptive immune response.

“Antisense activity” means any detectable and/or measurable change attributable to the hybridization of an antisense compound to its target nucleic acid. Antisense activity is a decrease in the amount or expression of a target nucleic acid or protein encoded by such target nucleic acid compared to target nucleic acid levels or target protein levels in the absence of the antisense compound.

“Antisense compound” means an oligomeric compound capable of achieving at least one antisense activity.

“alkyl” group refers to a saturated aliphatic hydrocarbon group containing 1-8 (e.g., 1-6 or 1-4) carbon atoms. An alkyl group can be straight or branched. Examples of alkyl groups include, but are not limited to, methyl, ethyl, propyl, isopropyl, butyl, isobutyl, sec-butyl, tert-butyl, n-pentyl, n-heptyl, or 2-ethylhexyl. An alkyl group can be substituted (i.e., optionally substituted) with one or more substituents such as halo; cycloaliphatic [e.g., cycloalkyl or cycloalkenyl]; heterocycloaliphatic [e.g., heterocycloalkyl or heterocycloalkenyl]; aryl; heteroaryl; alkoxy; aroyl; heteroaroyl; acyl [e.g., (aliphatic) carbonyl, (cycloaliphatic) carbonyl, or (heterocycloaliphatic) carbonyl]; nitro; cyano; amido [e.g., (cycloalkylalkyl) carbonylamino, arylcarbonylamino, aralkylcarbonylamino, (heterocycloalkyl) carbonylamino, (heterocycloalkylalkyl) carbonylamino, heteroarylcarbonylamino, heteroaralkylcarbonylamino alkylaminocarbonyl, cycloalkylaminocarbonyl, heterocycloalkylaminocarbonyl, arylaminocarbonyl, or heteroarylaminocarbonyl]; amino [e.g., aliphaticamino, cycloaliphaticamino, or heterocycloaliphaticamino]; sulfonyl [e.g., aliphatic-S(O)₂—]; sulfinyl; sulfanyl; sulfoxy; urea; thiourea; sulfamoyl; sulfamide; oxo; carboxy; carbamoyl; cycloaliphaticoxy; heterocycloaliphaticoxy; aryloxy; heteroaryloxy; aralkyloxy; heteroarylalkoxy; alkoxycarbonyl; alkylcarbonyloxy; or hydroxy. Without limitation, some examples of substituted alkyls include carboxyalkyl (such as HOOC-alkyl, alkoxycarbonylalkyl, and alkylcarbonyloxyalkyl); cyanoalkyl; hydroxyalkyl; alkoxyalkyl; acylalkyl; aralkyl; (alkoxyaryl)alkyl; (sulfonylamino)alkyl (such as alkyl-S(O)₂-aminoalkyl); aminoalkyl; amidoalkyl; (cycloaliphatic)alkyl; or haloalkyl.

“alkylene” refers to a bifunctional alkyl group.

A “bifunctional” moiety refers to a chemical group that is attached to the main chemical structure in two places, such as a linker moiety. Bifunctional moieties can be attached to the main chemical structure at any two chemically feasible substitutable points. Unless otherwise specified, bifunctional moieties can be in either direction, e.g. the bifunctional moiety “N—O” can be attached in the —N—O— direction or the —O—N— direction.

“Chemically distinct region” refers to a region of an antisense compound that is in some way chemically different than another region of the same antisense compound. For example, a region having 2′-O-methoxyethyl nucleotides is chemically distinct from a region having nucleotides without 2′-O-methoxyethyl modifications.

“Chimeric antisense compound” means an antisense compound that has at least two chemically distinct regions.

“Complementarity” means the capacity for pairing between nucleobases of a first nucleic acid and a second nucleic acid.

“Contiguous nucleobases” means nucleobases immediately adjacent to each other.

“Diluent” means an ingredient in a composition that lacks pharmacological activity, but is pharmaceutically necessary or desirable. For example, the diluent in an injected composition may be a liquid, e.g. saline solution.

“Dose” means a specified quantity of a pharmaceutical agent provided in a single administration, or in a specified time-period. In certain embodiments, a dose may be administered in one, two, or more boluses, tablets, or injections. For example, in certain embodiments where subcutaneous administration is desired, the desired dose requires a volume not easily accommodated by a single injection, therefore, two or more injections may be used to achieve the desired dose. In certain embodiments, the pharmaceutical agent is administered by infusion over an extended period-of-time or continuously. Doses may be stated as the amount of pharmaceutical agent per hour, day, week, or month.

“Chemically Modified” when referring to RNA or DNA bases, has its meaning understood in the art and includes a nucleoside base selected from the group consisting of 2′-substituted nucleoside, ‘—O-methoxyethyl” (also 2′-MOE and 2′-O(CH₂)₂—OCH₃), 2′-deoxynucleoside, 2′-O-methoxyethyl nucleotide, 5-methyl cytosine, monocylic nucleosides, Bicyclic nucleoside, 4′-2′ bicyclic nucleoside, 4′ to 2′ bicyclic nucleoside, locked nucleic acid, and Nucleoside mimetic, all as defined herein.

“Fully complementary” or “100% complementary” means each nucleobase of a first nucleic acid has a complementary nucleobase in a second nucleic acid. “Substantially Complementary” in reference to a Gapmer that is “substantially complementary” to the defined region on its target (SEQ ID NO. 1) means that no more than two nucleobases are not capable of hydrogen bonding with one another when the nucleobase sequence of the oligonucleotide and the other nucleic acid are aligned in opposing directions. Complementary nucleobases means nucleobases that are capable of forming hydrogen bonds with one another. For example, for a 10-mer oligonucleotide, a sequence which is substantially complementary can have 8, 9, or 10 bases which are complementary to a second nucleic acid. “Substantially complementary” includes “fully complementary” sequences where there are no bases which are mismatched within the oligonucleotide sequence to a second nucleic acid.

Complementary nucleobase pairs include adenine (A) and thymine (T), adenine (A) and uracil (U), cytosine (C) and guanine (G), 5-methyl cytosine (mC) and guanine (G). Complementary oligonucleotides and/or nucleic acids need not have nucleobase complementarity at each nucleoside. Rather, some mismatches are tolerated. “Fully complementary” or “100% complementary” in reference to oligonucleotides means that oligonucleotides are complementary to another oligonucleotide or nucleic acid at each nucleoside of the oligonucleotide.

“Contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

“Gapmer” means a modified oligonucleotide comprising an internal “gap” region having a plurality of DNA nucleosides positioned between external regions having one or more nucleosides, wherein the nucleosides comprising the internal region are chemically distinct from the nucleoside or nucleosides comprising the external regions. The internal region is often referred to as the “gap” and the external regions are often referred to as the “wings.” Unless otherwise indicated, “gapmer” refers to a sugar motif. Unless otherwise indicated, the sugar moieties of the nucleosides of the gap central region of a gapmer are unmodified 2′-deoxyribosyl. Thus, the term “MOE gapmer” indicates a gapmer having a sugar motif of 2′-MOE nucleosides in both wings and a gap of 2′-deoxynucleosides. Unless otherwise indicated, a MOE gapmer may comprise one or more modified internucleoside linkages and/or modified nucleobases and such modifications do not necessarily follow the gapmer pattern of the sugar modifications.

“Hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

“Immediately adjacent” means there are no intervening elements between the immediately adjacent elements.

“Modified nucleotide base” and “modified nucleoside” is a deoxyribose nucleotide or ribose nucleotide that is modified to have one or more chemical moieties not found in natural nucleic acids. Examples of modified nucleotide bases, and “modified nucleosides” are compounds of Formula Ia, Formula Ib, Formula IIa, or Formula IIb as described herein.

A “Non-bicyclic modified sugar moiety” is a sugar moiety of a modified nucleotide base, wherein the chemical modifications do not involve the transformation of the sugar moiety into a bicyclic or multicyclic ring system.

“Monocylic nucleosides” are nucleosides comprising modified sugar moieties that are not bicyclic sugar moieties. In certain embodiments, the sugar moiety, or sugar moiety analogue, of a nucleoside may be modified or substituted at any position.

“2′-modified sugar” means a furanosyl sugar modified at the 2′ position. Such modifications include substituents as described herein.

“Bicyclic nucleoside” (BNA) are modified nucleoside comprising a bicyclic sugar moiety. Examples of bicyclic nucleosides include without limitation nucleosides comprising a bridge between the 4′ and the 2′ ribosyl ring atoms. The synthesis of bicyclic nucleosides have been disclosed in, for example, 7,399,845, WO/2009/006478, WO/2008/150729, US2004-0171570, U.S. Pat. No. 7,427,672, Chattopadhyaya et al. J. Org. Chem. 2009, 74, 118-134, WO 99/14226, and WO 2008/154401. The synthesis and preparation of the methyleneoxy (4′-CH₂—O-2′) BNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). BNAs and their preparation are also described in WO98/39352 and WO 99/14226. Analogs of methyleneoxy (4′-CH₂—O-2′) BNA and 2′-thio-BNAs, have also been prepared (Kumar et al., Bioorg. Med. Chem. Lett., 1998, 8, 2219-2222). Preparation of locked nucleoside analogs comprising oligodeoxyribonucleotide duplexes as substrates for nucleic acid polymerases has also been described (WO 99/14226). Furthermore, synthesis of 2′-amino-BNA, a novel conformationally restricted high-affinity oligonucleotide analog has been described in the art (Singh et al., J. Org. Chem., 1998, 63, 10035-10039). In addition, 2′-amino- and 2′-methylamino-BNA's have been prepared and the thermal stability of their duplexes with complementary RNA and DNA strands has been previously reported. One carbocyclic bicyclic nucleoside having a 4′-(CH₂)₃-2′ bridge and the alkenyl analog bridge 4′-CH═CH—CH₂-2′ have been described (Freier et al., Nucleic Acids Research, 1997, 25 (22), 4429-4443 and Alback et al., J. Org. Chem., 2006, 71, 7731-7740). The synthesis and preparation of carbocyclic bicyclic nucleosides along with their oligomerization and biochemical studies have also been described (Srivastava et al., J. Am. Chem. Soc., 2007, 129 (26), 8362-8379).

A “4′-2′ bicyclic nucleoside” or “4′ to 2′ bicyclic nucleoside” is a bicyclic nucleoside comprising a furanose ring comprising a bridge connecting two carbon atoms of the furanose ring connects the 2′ carbon atom and the 4′ carbon atom of the sugar ring.

A “locked nucleic acid” (LNA) is a modified nucleotide base, wherein the chemical modifications are transformation of the sugar moiety into a bicyclic or multicyclic ring system. Two specific examples of locked nucleic acid compounds are β-D-methyleneoxy nucleotides, or “constrained methyl” (cMe) nucleotides; and β-D-ethyleneoxy nucleotides, or “constrained ethyl” (cEt) nucleotides.

“Mismatch” or “non-complementary” means a nucleobase of a first oligonucleotide that is not complementary with the corresponding nucleobase of a second oligonucleotide or target nucleic acid when the first and second oligonucleotide are aligned.

“Motif” means the pattern of unmodified and/or modified sugar moieties, nucleobases, and/or internucleoside linkages, in an oligonucleotide.

“Nucleobase” means an unmodified nucleobase or a modified nucleobase. An “unmodified nucleobase” is adenine (A), thymine (T), cytosine (C), uracil (U), and guanine (G). A “modified nucleobase” is a group of atoms other than unmodified A, T, C, U, or G capable of pairing with at least one unmodified nucleobase. A “5-methyl cytosine” is a modified nucleobase. A universal base is a modified nucleobase that can pair with any one of the five unmodified nucleobases. “Nucleobase sequence” means the order of contiguous nucleobases in a nucleic acid or oligonucleotide independent of any sugar or internucleoside linkage modification.

“Nucleoside” means a compound comprising a nucleobase and a sugar moiety. The nucleobase and sugar moiety are each, independently, unmodified or modified. “Modified nucleoside” means a nucleoside comprising a modified nucleobase and/or a modified sugar moiety. Modified nucleosides include abasic nucleosides, which lack a nucleobase. “Linked nucleosides” are nucleosides that are connected in a contiguous sequence (i.e., no additional nucleosides are presented between those that are linked).

“Nucleoside mimetic” includes those structures used to replace the sugar or the sugar and the base and not necessarily the linkage at one or more positions of an oligomeric compound such as for example nucleoside mimetics having morpholino, cyclohexenyl, cyclohexyl, tetrahydropyranyl, bicyclo or tricyclo sugar mimetics, e.g., non-furanose sugar units. Nucleotide mimetic includes those structures used to replace the nucleoside and the linkage at one or more positions of an oligomeric compound such as for example peptide nucleic acids or morpholinos (morpholinos linked by —N(H)—C(═O)—O— or other non-phosphodiester linkage). Sugar surrogate overlaps with the slightly broader term nucleoside mimetic but is intended to indicate replacement of the sugar unit (furanose ring) only. The tetrahydropyranyl rings provided herein are illustrative of an example of a sugar surrogate wherein the furanose sugar group has been replaced with a tetrahydropyranyl ring system.

“Parenteral administration” means administration through injection (e.g., bolus injection) or infusion. Parenteral administration includes subcutaneous administration, intravenous administration, intramuscular administration, intraarterial administration, intraperitoneal administration, or intracranial administration, e.g., intrathecal or intracerebroventricular administration.

“Pharmaceutically acceptable carriers” means physiologically and pharmaceutically acceptable carriers of compounds. Pharmaceutically acceptable carriers retain the desired biological activity of the parent compound and do not impart undesired toxicological effects thereto.

“Phosphorothioate linkage” means a linkage between nucleosides where the phosphodiester bond is modified by replacing one of the non-bridging oxygen atoms with a sulfur atom.

“Reducing or inhibiting the amount or activity” refers to a reduction or blockade of the transcriptional expression or activity relative to the transcriptional expression or activity in an untreated or control sample and does not necessarily indicate a total elimination of transcriptional expression or activity.

“Self-complementary” in reference to an oligonucleotide means an oligonucleotide that at least partially hybridizes to itself.

“Side effects” means physiological responses attributable to a treatment other than the desired effects. Side effects include injection site reactions, liver function test abnormalities, renal function abnormalities, liver toxicity, renal toxicity, central nervous system abnormalities, myopathies, and malaise. For example, increased aminotransferase levels in serum may indicate liver toxicity or liver function abnormality. For example, increased bilirubin may indicate liver toxicity or liver function abnormality.

“Single-stranded oligonucleotide” means an oligonucleotide which is not hybridized to a complementary strand.

“Specifically hybridizable” means an antisense compound having a sufficient degree of complementarity between an antisense oligonucleotide and a target nucleic acid to induce a desired effect, while exhibiting minimal or no effects on non-target nucleic acids under conditions in which specific binding is desired, i.e., under physiological conditions in the case of in vivo assays and therapeutic treatments.

“Sugar moiety” means an unmodified sugar moiety or a modified sugar moiety. As used herein, “unmodified sugar moiety” means a 2′-OH(H) ribosyl moiety, as found in RNA (an “unmodified RNA sugar moiety”), or a 2-H(H) deoxyribosyl moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. As used herein, “modified sugar moiety” or “modified sugar” means a modified furanosyl sugar moiety or a sugar surrogate.

“Sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or target nucleic acids.

“Targeting” or “targeted” means the process of design and selection of an antisense compound that will specifically hybridize to a target nucleic acid and induce a desired effect.

“Target segment” means the sequence of nucleotides of a target nucleic acid to which an antisense compound is targeted. “5′ target site” refers to the 5′-most nucleotide of a target segment. “3′ target site” refers to the 3′-most nucleotide of a target segment.

“Target nucleic acid” and “target RNA” mean a nucleic acid that an antisense compound is designed to affect, such as AMANZI.

“Target region” means a portion of a target nucleic acid to which an oligomeric compound is designed to hybridize.

“Unmodified nucleotide” means a nucleotide composed of naturally occurring nucleobases, sugar moieties, and internucleoside linkages. An unmodified nucleotide is an RNA nucleotide (i.e. β-D-ribonucleosides) or a DNA nucleotide (i.e. β-D-deoxyribonucleoside).

### Antisense ASO Compounds

The present disclosure provides a gapmer antisense oligonucleotide (ASO) compound (“gapmer compound”) that is complementary (for example, from about 91% complementary to about 100% complementary, including 100% complementary over the entire length of the gapmer compound) to a region of AMANZI long non-coding RNA, and that inhibits multiple acute inflammatory gene transcription regulated by the AMANZI long non-coding RNA. In various embodiments of the present disclosure, a gapmer compound comprises a modified oligonucleotide of 12 to 29 linked nucleosides in length. The gapmer compound is substantially complementary (for example, having no more than one nucleotide mismatch over the entire length of the gapmer compound) to a region of AMANZI (SEQ ID NO. 1), and inhibits multiple acute inflammatory gene transcription from being regulated by the AMANZI long non-coding RNA. The gapmer compound comprises: (a) a 5′ wing sequence having from about three to about seven wing modified nucleotide bases; (b) a central gap region sequence having from about six to about fifteen 2′ deoxynucleotides; and (c) a 3′ wing sequence having from about three to about seven wing modified nucleotide bases; wherein the gapmer compound nucleotides are each linked with phosphorothioate internucleoside linkages, phosphorothiolate internucleoside linkages, or combinations thereof over the entire length of the gapmer compound; and wherein the modified nucleotide base modifications are selected from the group consisting of a 2′-methoxyethyl (MOE) modification, a locked nucleic acid (LNA) modification (for example, a modified sugar containing a cMe or cEt), a 2′F-ANA modification, a 2′-O-methoxyethyl (2′OMe) modification, or combinations thereof.

The chemical modification of antisense oligonucleotides may enhance their resistance to nucleases and may enhance their ability to enter cells. For example, phosphorothioate oligonucleotides may be used. Other deoxynucleotide analogs include methylphosphonates, phosphoramidates, phosphorodithioates, N3′P5′-phosphoramidates and oligoribonucleotide phosphorothioates and their 2′-O-alkyl analogs and 2′-O-methylribonucleotide methylphosphonates. Alternatively mixed backbone oligonucleotides (“MBOs”) may be used. MBOs contain segments of phosphothioate oligodeoxynucleotides and appropriately placed segments of modified oligodeoxy- or oligoribonucleotides. MBOs have segments of phosphorothioate linkages and other segments of other modified oligonucleotides, such as methylphosphonate, which is non-ionic, and very resistant to nucleases or 2′-O-alkyloligoribonucleotides.

In some embodiments, an oligonucleotide sugar moiety is a modified sugar moiety. In some embodiments, the modified sugar moiety can be a sugar moiety which is a conformationally-strained sugar. In some embodiments, the conformationally-strained sugar can be a locked nucleotide (locked nucleic acid, or LNA). In some embodiments, the locked nucleotide can be selected from one of the following types: 2′-O—CH₂-4′ (oxy-LNA), 2′-CH₂—CH₂-4′ (methylene-LNA), 2′-NH—CH₂-4′ (amino-LNA), 2′-N(CH₃)—CH₂-4′ (methylamino-LNA), 2′-S—CH₂-4′ (thio-LNA), and 2′-Se—CH₂-4′ (seleno-LNA). In some embodiments, the conformationally-strained sugar can be a bridged nucleic acid (BNA). Some conformationally-strained sugar can be a locked nucleic acid are shown in Formula III and Formula IV in U.S. Pat. No. 10,465,188, herein incorporated by reference.

Synthesis of antisense oligonucleotides can be performed. Sec e.g. Stein C. A. and Krieg A. M. (eds), Applied Antisense Oligonucleotide Technology, 1998 (Wiley-Liss).

### Antisense Compound Motifs

In a gapmer an internal region having a plurality of nucleotides or linked nucleosides is positioned between external regions having a plurality of nucleotides or linked nucleosides that are chemically distinct from the nucleotides or linked nucleosides of the internal region. In the case of an antisense oligonucleotide having a gapmer motif, the gap segment generally serves as the substrate for endonuclease cleavage, while the wing segments comprise modified nucleosides. The regions of a gapmer (5′ wing, gap sequence, and 3′ wing) are differentiated by the types of sugar moieties comprising each distinct region. The types of sugar moieties that are used to differentiate the regions of a gapmer may include β-D-ribonucleosides, β-D-deoxyribonucleosides, 2′-modified nucleosides (such 2′-modified nucleosides may include 2′-MOE, and 2′-O—CH₃, among others), and bicyclic sugar modified nucleosides (such bicyclic sugar modified nucleosides may include those having a 4′-(CH₂)ₙ—O-2′ bridge, where n=1 or n=2). Preferably, each distinct region comprises uniform sugar moieties. The wing-gap-wing motif is frequently described as “X-Y-Z”, where “X” represents the length of the 5′ wing region, “Y” represents the length of the gap region, and “Z” represents the length of the 3′ wing region. In general, a gapmer described as “X-Y-Z” has a configuration such that the gap segment is positioned immediately adjacent each of the 5′ wing segment and the 3′ wing segment. Thus, no intervening nucleotides exist between the 5′ wing segment and gap segment, or the gap segment and the 3′ wing segment. Often, X and Z are the same chemistry of modified nucleobase, or they are different. Preferably, Y is between 8 and 15 nucleotides. X or Z can be any of 3, 4, 5, 6, 7, 8, 9, or 10 nucleotides. Thus, gapmers include, but are not limited to, for example 5-10-5, 4-8-4, 4-12-3, 4-12-4, 3-14-3, 2-13-5, 2-16-2, 1-18-1, 3-10-3, 2-10-2, 1-10-1, 2-8-2, 6-8-6 or 5-8-5.

In a preferred embodiment, a gapmer has a gap segment of ten 2′-deoxyribonucleotides positioned immediately adjacent to and between wing segments of five chemically modified nucleosides. In certain embodiments, the chemical modification in the wings comprises a 2′-sugar modification. In another embodiment, the chemical modification comprises a 2′-MOE sugar modification. Preferably, a gap-widened antisense oligonucleotide has a gap segment of eight 2′-deoxyribonucleotides positioned immediately adjacent to and between wing segments of five chemically modified nucleosides. Or the chemical modification comprises a 2′-sugar modification. Or the chemical modification comprises a 2′-MOE sugar modification.

A gapmer has a gap segment of eight 2′-deoxyribonucleotides positioned immediately adjacent to and between wing segments of five to six chemically modified nucleosides. The chemical modification comprises a 2′-sugar modification, such as a 2′-MOE sugar modification.

### Hybridization

Hybridization occurs between a gapmer compound and a target AMANZI nucleic acid (SEQ ID NO. 1). The most common mechanism of hybridization involves hydrogen bonding (e.g., Watson-Crick, Hoogsteen or reversed Hoogsteen hydrogen bonding) between complementary nucleobases of the nucleic acid molecules. Hybridization can occur under varying conditions. Stringent conditions are sequence-dependent and are determined by the nature and composition of the nucleic acid molecules to be hybridized.

### Modified Sugar Moieties

Antisense compounds can optionally contain one or more nucleosides wherein the sugar group has been modified. Such sugar modified nucleosides may impart enhanced nuclease stability, increased binding affinity, or some other beneficial biological property to the antisense compounds. Nucleosides comprise chemically modified ribofuranose ring moieties. Examples of chemically modified ribofuranose rings include, without limitation, addition of substituent groups (including 5′ and 2′ substituent groups, bridging of non-geminal ring atoms to form bicyclic nucleic acids (BNA), replacement of the ribosyl ring oxygen atom with S, N(R), or C(R₁) (R₂) (R, R₁ and R₂ are each independently H, C₁-C₁₂ alkyl or a protecting group) and combinations thereof. Examples of chemically modified sugars include 2′-F-5′-methyl substituted nucleoside (WO2008/101157 for other disclosed 5′,2′-bis substituted nucleosides) or replacement of the ribosyl ring oxygen atom with S with further substitution at the 2′-position (U.S. Patent Application 2005/0130923) or alternatively 5′-substitution of a BNA (WO2007/134181 wherein LNA is substituted with for example a 5′-methyl or a 5′-vinyl group).

Antisense oligonucleotides may be part of compositions which may comprise oligonucleotides to more than one region of AMANZI (SEQ ID NO: 1). Preferably, one AMANZI transcriptional inhibitor gapmer compound is an oligonucleotide having the sequence of all or 1 or 2 base mismatches of that of the sequence of SEQ ID NO: 41-46, 63-64, 66, 67, or 101-104.

When specific proteins are referred to herein, derivatives, variants, and fragments are contemplated and included. Protein derivatives and variants are well understood to those of skill in the art and can involve insertional, substitutional or deletional amino acid sequence variants known in the art.

### Modified Nucleotide Bases

Modified nucleotide bases include Formula Ia Formula Ib, Formula IIa, or Formula IIb:

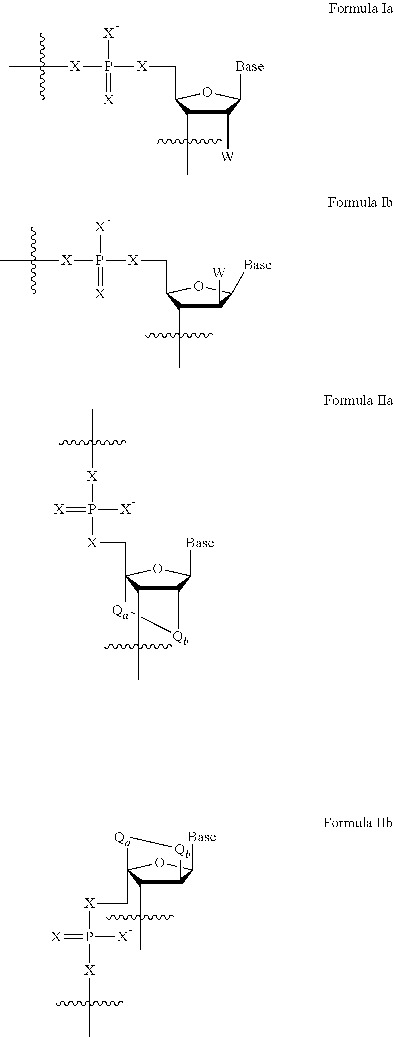

wherein
   each X is independently O or S, wherein 0, 1, or 2 instances of X is S;
   each W is independently H, OH, halo, or —O—C₁₋₆ alkyl, wherein the alkyl is optionally substituted with up to three instances of C₁₋₄ alkyl, C₁₋₄ alkoxy, halo, amino, CN, NO₂, or OH; each Qₐ is independently a bifunctional C₁₋₆ alkylene, optionally substituted with up to two instances of C₁₋₄ alkyl, C₁₋₄ alkoxy, halo, or OH; and each Q_{b} is independently a bond or a bifunctional moiety selected from —O—, —S—, —N—O—, —N(R)—, —C(O)—, —C(O)O—, and —C(O)N(R)—, wherein R is an unsubstituted C₁₋₄ alkyl.

In one embodiment, each X is O. In another embodiment, one instance of X is S.

In one embodiment, the gapmer comprises one or more nucleotides of Formula Ia or Formula Ib, wherein W is halo. In a further embodiment, W is fluoro. In another further embodiment, the gapmer comprises one or more nucleotides of Formula Ia. In another further embodiment, the gapmer comprises one or more nucleotides of Formula Ib.

In one embodiment, the gapmer comprises one or more nucleotides of Formula Ia or Formula Ib, wherein W is —O—C₁₋₆ alkyl, wherein the alkyl is optionally substituted with up to three instances of C₁₋₄ alkyl, C₁₋₄ alkoxy, halo, amino, or OH. In a further embodiment, W is —O—C₁₋₆ alkyl, wherein the alkyl is optionally substituted with C₁₋₄ alkoxy. In a further embodiment, W is an unsubstituted —O—C₁₋₆ alkyl. In another further embodiment, W is —O—C₁₋₆ alkyl, wherein the alkyl is substituted with C₁₋₄ alkoxy. In a further embodiment, W is selected from methoxy and —O—CH₂CH₂—OCH₃. In one embodiment, the gapmer comprises one or more nucleotides of Formula Ia. In another embodiment, the gapmer comprises one or more nucleotides of Formula Ib.

In one embodiment, the gapmer comprises one or more β-D nucleotides of Formula IIa or α-L nucleotides of Formula IIb, wherein Qₐ is an unsubstituted bifunctional C₁₋₆ alkylene, and Q_{b} is a bond or a bifunctional moiety selected from —O—, —S—, —N—O—, and —N(R)—. In a further embodiment, Qₐ is selected from —CH₂—, —CH₂—CH₂—, —CH(CH₃)—, —CH₂—CH₂(CH₃)—, and Q_{b} is a bond or a bifunctional moiety selected from —O—, —S—, —N(R)—O—, and —N(R)—, wherein R is H or C₁₋₆ alkyl.

In one embodiment of Formula IIa or Formula IIb, Qₐ is —CH₂— and Q_{b} is —O—. In another embodiment of Formula IIa or Formula IIb, Qₐ is —CH₂—CH₂— and Q_{b} is —O—. In another embodiment of Formula IIa or Formula IIb, Qₐ is —CH₂— and Q_{b} is —N(R)—O—, wherein R is H or C₁₋₆ alkyl. In another embodiment of Formula IIa or Formula IIb, Qₐ is —CH(CH₃)— and Q_{b} is —O—. In another embodiment of Formula IIa or Formula IIb, Qₐ is —CH₂— and Q_{b} is —S—. In another embodiment of Formula IIa or Formula IIb, Qₐ is —CH₂— and Q_{b} is —N(R)—, wherein R is H or C₁₋₆ alkyl. In another embodiment of Formula IIa or Formula IIb, Qₐ is —CH₂—CH(CH₃)— and Q_{b} is a bond.

In some embodiments, the gapmer comprises one or more nucleotides selected from the following modified nucleotides:

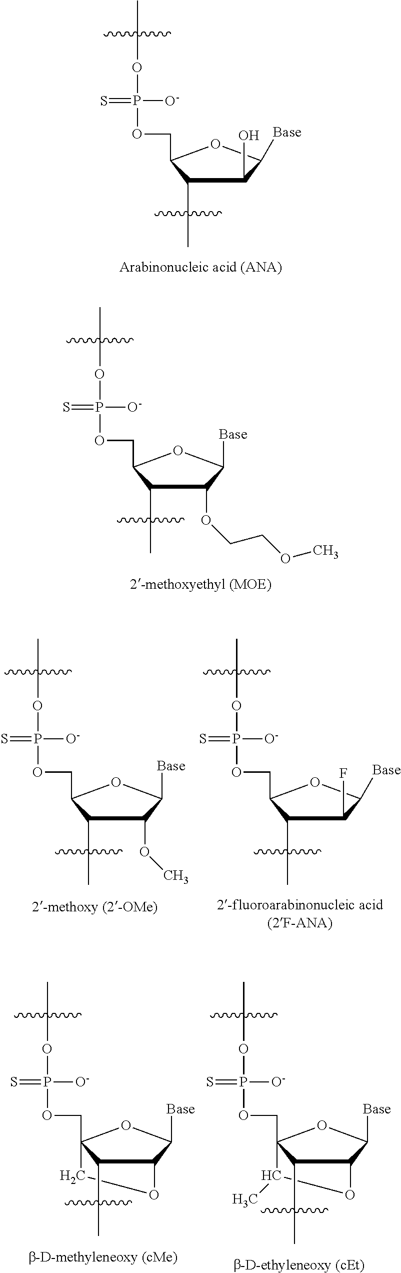

Many other bicyclo and tricyclo sugar surrogate ring systems can be used to modify nucleosides for incorporation into antisense compounds (see, for example, review article: Leumann, Bioorg. Med. Chem., 2002, 10, 841-854).

### AMANZI Transcription Modulators

Any transcription regulator that is capable of eliciting a desired inhibition of the protein expression (e.g., transcription) of the AMANZI gene region, and as well as AMANZI transcriptional inhibitor gapmer compounds of this disclosure, may be used in embodiments of the invention as described herein. Any AMANZI transcriptional inhibitor gapmer compound that modulates the AMANZI gene region are also provided in particular embodiments (e.g., those that modulate, block or lessen the transcription of AMANZI).

### Administration

The gapmers described herein may be administered in a number of ways depending upon whether local or systemic treatment is desired and upon the area to be treated. Administration may be topical, pulmonary, e.g., by inhalation or insufflation of powders or aerosols, including by nebulizer; intratracheal, intranasal, epidermal and transdermal, oral or parenteral. The compounds and compositions described herein can be delivered in a manner to target a particular tissue, such as the bone marrow or brain. The compounds and compositions described herein are administered parenterally. “Parenteral administration” means administration through injection or infusion. Parenteral administration includes subcutaneous administration, intravenous administration, intramuscular administration, intraarterial administration, intraperitoneal administration, or intracranial administration, e.g. intracerebral administration, intrathecal administration, intraventricular administration, ventricular administration, intracerebroventricular administration, cerebral intraventricular administration or cerebral ventricular administration. Administration can be continuous, or chronic, or short or intermittent.

Parenteral administration is also by infusion. Infusion can be chronic or continuous or short or intermittent, with a pump or by injection. In some embodiments, parenteral administration is subcutaneous.

### Formulations

The pharmaceutical compositions of this invention include, for example, compositions comprising an AMANZI transcriptional inhibitor gapmer compound. The pharmaceutical formulations of this invention may further comprise one or more pharmaceutically acceptable excipients.

The pharmaceutical formulations of this invention may further comprise one or more pharmaceutically acceptable excipients.

The AMANZI transcriptional inhibitor gapmer compounds may be present in the formulation in a substantially isolated form. It will be understood that the product may be mixed with carriers or diluents that will not interfere with the intended purpose of the product and still be regarded as substantially isolated. A product of the invention may also be in a substantially purified form, in which case it will generally comprise about 80%, 85%, or 90%, e.g. at least about 88%, at least about 90, 95 or 98%, or at least about 99% of a oligonucleotide, or dry mass of the preparation.

Pharmaceutically acceptable diluents, carriers and/or excipients include those suitable for veterinary use as well as human pharmaceutical use. By way of example, diluents, carriers and/or excipients include solutions, solvents, dispersion media, delay agents, polymeric and lipidic agents, emulsions and the like. By way of further example, suitable liquid carriers, especially for injectable solutions, include water, aqueous saline solution, aqueous dextrose solution, and the like, and vehicles such as liposomes being also especially suitable for administration of agents.

Suitable carriers and diluents include buffered, aqueous solutions, saline, dextrose, glycerol, isotonic saline solutions, for example phosphate-buffered saline, isotonic water, and the like and combinations thereof. In some embodiments, carriers may include propylene glycol, dimethyl isosorbide, and water, and even more particularly, phosphate buffered saline, isotonic water, deionized water, monofunctional alcohols and symmetrical alcohols. In some embodiments pharmaceutically acceptable carrier or diluent may be or contain a thermosetting poloxamer (which may be a liquid or gel, depending on the temperature), a carboxycellulose (e.g. carboxymethylcellulose), a collagen (e.g., a Type I collagen), a collagenous material comprising tropocollagen, a hyaluronan or derived-hyaluronic acid, and/or an oil (e.g., Emu oil). Suitable carriers can be large, slowly metabolized macromolecules such as proteins, polysaccharides, polylactic acids, polyglycolic acids, polymeric amino acids, and amino acid copolymers.

Compositions may take the form of any standard known dosage form including tablets, pills, capsules, semisolids, powders, sustained release formulation, solutions, suspensions, elixirs, aerosols, liquids for injection, gels, creams, transdermal delivery devices (for example, a transdermal patch), inserts such as ocular inserts, or any other appropriate compositions.

Preferably the AMANZI transcriptional inhibitor gapmer compound is combined with a pharmaceutically acceptable carrier or diluent to produce a pharmaceutical composition.

Pharmaceutically acceptable salts can also be present, e.g., mineral acid salts such as hydrochlorides, hydrobromides, phosphates, sulfates, and the like; and the salts of organic acids such as citrates, acetates, propionates, malonates, benzoates, and the like.

In addition, if desired substances such as wetting or emulsifying agents, stabilizing or pH buffering agents, or preservatives may also be present. In some embodiments, the pharmaceutical compositions of this invention will comprise suitable ophthalmically acceptable buffers, such as acetate buffers, citrate buffers, phosphate buffers, borate buffers and mixtures thereof. In some embodiments, the buffers useful in the present invention include boric acid, sodium borate, sodium phosphates, including mono, di- and tri-basic phosphates, such as sodium phosphate monobasic monohydrate and sodium phosphate dibasic heptahydrate, and mixtures thereof. In some embodiments, the preservative may be stabilized chlorine dioxide, cationic polymers or quaternary ammonium compounds. In some embodiments the pharmaceutical compositions may also comprise wetting agents, nutrients, viscosity builders, antioxidants, and the like, for example, disodium ethylene diamine tetraacetate, alkali metal hexametaphosphate, citric acid, sodium citrate, sodium metabisulfite, sodium thiosulfate, N-acetylcysteine, butylated hydroxyanisole, butylated hydroxytoluene, polyvinyl alcohol, polyoxamers, polyvinyl pyrrollidone, hydroxypropyl methyl cellulose, hydroxyethylmethyl cellulose, and mixtures thereof and mixtures thereof. In some embodiments, the pharmaceutical formulations of this invention will not include a preservative. In some embodiments, the AMANZI transcriptional inhibitor gapmer composition or formulation comprises sodium phosphate dibasic heptahydrate or potassium phosphate, monobasic or both.

Uptake of nucleic acids by mammalian cells may be enhanced by the of known transfection techniques including the use of transfection agents. Such techniques may be used with certain AMANZI transcriptional inhibitor gapmer compounds. Examples of useful transfection agents include cationic agents (for example calcium phosphate and DEAE-dextran) and lipofectants (for example Lipofectam™ and Transfectam™), and surfactants.

The compositions may be formulated in accordance with standard techniques known in the art, including those as may be found in such standard references as Gennaro AR: Remington: The Science and Practice of Pharmacy, 20^{th} ed., Lippincott, Williams & Wilkins, 2000, for example.

Any container suitable for storing and/or administering a pharmaceutical composition may be used in a combination product of the invention. Suitable containers will be appreciated by persons skilled in the art. By way of example, such containers include vials and syringes. The containers may be suitably sterilized and hermetically scaled.

Such compositions comprise a pharmaceutically acceptable solvent, such as water or saline, diluent, carrier, or adjuvant. The pharmaceutical compositions may be administered in a number of ways depending upon whether local or systemic treatment is desired and upon the area to be treated. Administration may be topical (including ophthalmic and to mucous membranes including vaginal and rectal delivery), pulmonary, by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial (intrathecal or intraventricular, administration).

The compounds may also be admixed, conjugated or otherwise associated with other molecules, molecule structures or mixtures of compounds, as for example, liposomes, receptor-targeted molecules, or other formulations, for assisting in uptake, distribution and/or absorption.

The term “pharmaceutically acceptable carriers” refers to physiologically and pharmaceutically acceptable carriers of the compounds i.e., carriers that retain the desired biological activity of the parent compound and do not impart undesired toxicological effects thereto. For oligonucleotides, preferred examples of pharmaceutically acceptable carriers and their uses are further described in U.S. Pat. No. 6,287,860, which is incorporated by reference herein. Sodium carriers have been shown to be suitable forms of oligonucleotide drugs.

Formulations include liposomal formulations. The term “liposome” means a vesicle composed of amphiphilic lipids arranged in a spherical bilayer or bilayers. Liposomes are unilamellar or multilamellar vesicles which have a membrane formed from a lipophilic material and an aqueous interior that contains the composition to be delivered. Cationic liposomes are positively charged liposomes which are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes also include “sterically stabilized” liposomes which refers to liposomes comprising one or more specialized lipids that, when incorporated into liposomes, result in enhanced circulation lifetimes relative to liposomes lacking such specialized lipids. Liposomes and their uses are further described in U.S. Pat. No. 6,287,860, which is incorporated by reference herein.

Preferred formulations for topical administration include those in which the oligonucleotides are in admixture with a topical delivery agent such as lipids, liposomes, fatty acids, fatty acid esters, steroids, chelating agents and surfactants. Preferred lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA).

### Lipid Nanoparticles

LNPs are multi-component systems that typically consist of an ionizable amino lipid, a phospholipid, cholesterol, and a polyethylene glycol (PEG)-lipid, with all of the components contributing to efficient delivery of the nucleic acid drug cargo and stability of the particle (Schroeder et al., J. Intern. Med. 2010; 267:9-21). The cationic lipid electrostatically condenses the negatively charged RNA into nanoparticles and the use of ionizable lipids that are positively charged at acidic pH is thought to enhance endosomal escape. Formulations for delivery, both clinically and non-clinically, are predominantly based on cationic lipids such as DLin-MC3-DMA (MC3). (Kanasty et al. Nat. Mater. 2013; 12:967-977; and Xue et al. Curr. Pharm. Des. 2015; 21:3140-3147).

Further LNP's include a nanoemulsion having a perfluorcarbon component (a) consisting of at least one least one perfluorcarbon compound, an emulsifying component (b) such as phospholipids and optionally helper lipids, and an endocytosis enhancing component (c) that comprises at least one compound inducing cellular uptake of the nanoemulsion. A perfluorcarbon compound of component (a) is preferably selected from compounds having the structure CₘF₂ₘ₊₁X, XCₘF₂ₘX, XCₙF₂ₙOCₒF₂ₒX, N(CₒF₂ₒX)₃ and N(CₒF₂ₒ+1)₃, wherein m is an integer from 3 to 10, n and o are integers from 1 to 5, and X is independently from further occurrence selected from Cl, Br and I. Examples of perfluorcarbon compounds are perfluorooctyl bromide and perfluorotributylamine.

Examples of the emulsifying agents include phospholipids, such as the phospholipid compound represented by the formula I:

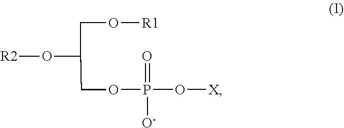

wherein, R¹ and R² are independently selected from H and C₁₆₋₂₄ acyl residues, which may be saturated or unsaturated and may carry 1 to 3 residues R³ and wherein one or more of the C-atoms may be substituted by O or NR⁴, and X is selected from H, —(CH₂)ₚ—N(R⁴)₃⁺, —(CH₂)ₚ—CH(N(R⁴)₃⁺)—COO, —(CH₂)ₚ—CH(OH)—CH₂OH and —CH₂ (CHOH)ₚ—CH₂OH (wherein p is an integer from 1 to 5); R³ is independently selected from H, lower alkyl, F, Cl, CN und OH; and R⁴ is independently selected from H, CH₃ und CH₂CH₃, or a pharmacologically acceptable carrier thereof.

Following subcutaneous (s.c.) administration, LNPs and their mRNA cargo are expected to be largely retained at the site of injection, resulting in high local concentrations. Since LNPs are known to be pro-inflammatory, largely attributed to the ionizable lipid present in the LNPs, (Sabnis et al. Mol. Ther. 2018; 26:1509-1519) then it would be expected that s.c. administration of mRNA formulated in LNPs would be associated with dose-limiting inflammatory responses. Co-administration of dexamethasone with LNP reduces the immune-inflammatory response following i.v. administration (Abrams et al. Mol. Ther. 2010; 18:171-180). And Chen et al. (J. Control. Release. 2018; 286:46-54.) showed reduced immune stimulation following systemic administration by incorporating lipophilic dexamethasone prodrugs within LNP-containing nucleic acids.

### Dosing

Optimal dosing schedules are calculated from measurements of drug accumulation in the body of the patient. Optimum dosages vary depending on the relative potency of individual oligonucleotides, and can generally be estimated based on EC50s found to be effective in in vitro and in vivo animal models. In general, dosage is from 0.01 μg to 100 g per kg of body weight, and may be given once or more daily, weekly, monthly or yearly, or at desired intervals. Following successful treatment, it may be desirable to have the patient undergo maintenance therapy to prevent the recurrence of the disease state, wherein the oligonucleotide is administered in maintenance doses, ranging from 0.01 μg to 100 g per kg of body weight, once or more daily.

Therapeutically effective amounts include but are not limited to the doses described herein. Described doses and other therapeutically effective amounts are administered in one or more of the therapeutically effective dose regimens described herein.

### Articles of Manufacture/Kits

In another embodiment of the invention, an article of manufacture, or “kit”, containing materials useful for inhibiting AMANZI is provided. The kit comprises a container with a composition comprising one or more modulators, e.g. an AMANZI modulator such as a AMANZI transcriptional inhibitor gapmer compound, for example. Suitable containers include, e.g., bottles, vials, etc. The container may be formed from a variety of materials such as glass or plastic. The kit may also comprise a pharmaceutically acceptable carrier. In some embodiments the kit may also include components for administering the compositions, for example, a syringe, needle, microneedle, etc.

### Manufacture

The oligonucleotides of this invention can be manufactured using solid-phase chemistries for synthesizing oligonucleotides, chemistries known in the art for synthesizing and preparing peptides and peptidomimetics, and chemistries know in the art for synthesizing organic compounds. In one aspect, the formulations of this invention will comprise a salt of the oligonucleotides of this invention, such as the sodium salt of the oligonucleotides of this invention. The kit may also comprise a pharmaceutically acceptable carrier. In one embodiment the formulation may comprise the sodium salt of an gapmer ASO compound as described herein.

In some embodiments, the formulations of this invention are substantially pure. By substantially pure is meant that the formulations comprise less than about 10%, 5%, or 1%, and preferably less than about 0.1%, of any nucleotide or non-nucleotide impurity. In some embodiments the total impurities, including metabolites of the AMANZI transcriptional inhibitor gapmer type of ASO (antisense oligonucleotide) compound, will be not more than 15%. In some embodiments the total impurities, including metabolites of the AMANZI transcriptional inhibitor gapmer type of ASO (antisense oligonucleotide) compound, will be not more than 12%. In some embodiments the total impurities, including metabolites of the AMANZI transcriptional inhibitor gapmer type of ASO (antisense oligonucleotide) compound, will be not more than 11%. In other embodiments the total impurities, including metabolites of the AMANZI transcriptional inhibitor gapmer type of ASO (antisense oligonucleotide) compound, will be not more than 10%.

Sterile compositions comprising the AMANZI transcriptional inhibitor gapmer type of ASO (antisense oligonucleotide) compounds of this invention prepared using aseptic processing by dissolving the AMANZI transcriptional inhibitor gapmer type of ASO (antisense oligonucleotide) compound in the formulation vehicle. In one embodiment, the formulation may also be sterilized by filtration. Excipients used in the manufacture of the formulations of this invention are widely used in pharmaceutical products and released to Pharmacopeial standards.

### EXAMPLES

### Example 1

This example provides an in vitro screening system of candidate gapmers for inhibiting gene transcription regulated by long non-coding RNA AMANZI. The effect of candidate gapmer compounds were screened for target nucleic acid expression (e.g., messenger RNA) by RT-PCR.

THP-1 human monocytic cell line (derived from an acute leukemia patient) was obtained from InvivoGen. THP1 cells were maintained in complete media, which is composed of RPMI 1640, 1% (2 mM) GlutaMAX L-glutamine supplement, 25 mM HEPES, 10% FBS, 100 μg/ml Normocin, Pen-Strep (100 U/ml), Blasticidin (10 μg/ml) and Zeocin (100 μg/ml). Prior to seeding for the screen, the THP-1 monocyte culture was split by 50% to enable the cells to re-enter an exponential growth phase. 250,000 cells were seeded per well in quadruplicate in 96-well plates with 180 μL of complete medium in each well. Each gapmer compound tested was added to the THP-1 cells at a final concentration of 10 μM and mixed gently. Plates were incubated at 37° C. at 5% CO2 for 24 hours. Then, LPS (10 ng/mL) was added to each well, and plates were incubated at 37° C. at 5% CO2 for another 24 hours.

Antisense modulation of AMANZI expression on specified genes was assayed by real-time PCR (RT-PCR). RNA analysis was performed on total cellular RNA or poly(A)+mRNA.

RNA was isolated and prepared using TRIZOL® Reagent (Thermo Fisher Scientific) and Direct-zol RNA Miniprep Kit (Zymo Research) according to the manufacturer's recommended protocols.

Quantitation of target RNA levels was accomplished by quantitative real-time PCR using a CFX Real-time qPCR detection system (Bio Rad). Prior to real-time PCR, the isolated RNA was subjected to a reverse transcriptase (RT) reaction, which produces complementary DNA (cDNA) that is then used as the substrate for the real-time PCR amplification. RT reaction reagents and real-time PCR reagents were obtained from Thermo Fisher Scientific, and protocols for their use are provided by the manufacturer. Gene (or RNA) target quantities obtained by real time PCR were normalized using expression levels of stably expressed housekeeping genes such as HPRT or RPL37A. Total RNA was quantified using a Qubit Fluorometer (Invitrogen/Scientific) and a Qubit RNA HS Assay Kit (Thermo Fisher Scientific Cat. No. Q32852) in accordance with the manufacturer's protocol. The Qubit Flourometer was calibrated with standards.

A series of assayed gapmer compounds are shown in Table 1. These gapmers were designed to target different regions of the human AMANZI long non-coding RNA (SEQ ID NO. 1). The gapmer compounds in Table 1 are chimeric oligonucleotides (“gapmers”) having different configurations. For instance, gapmers with a configuration of 20 (5-10-5) nucleotides in length were composed of a central “gap” region of ten 2′-deoxynucleotides, which was flanked on both sides (5′ and 3′ directions) by five-nucleotide “wings”. These wings were composed of 2′-methoxyethyl (2′-MOE) sugar modified nucleosides. The internucleotide (backbone) linkages were phosphorothioate throughout the entire oligonucleotide sequence. Cytidine residues were 5-methylcytidines unless indicated otherwise, in which case they were cytidines residues. Gapmers with a configuration of 16 (3-10-3) nucleotides in length were composed of a central “gap” region comprising ten 2′-deoxynucleotides, which was flanked on both sides (5′ and 3′ directions) by three-nucleotide “wings”. In some embodiments, the wings were composed of locked nucleic acid (LNA) modified nucleosides employing a cMe locked nucleic acid modification. The internucleotide (backbone) linkages were phosphorothioate throughout the entire oligonucleotide sequence. Cytidine residues were 5-methylcytidines unless indicated otherwise, in which case they were cytidine residues.

Gapmer compounds used are shown in Table 1. Abbreviations of the nucleoside modification in Table 1: M=2′-methoxyethyl (2′-MOE) modified nucleoside; L=locked nucleic acid (LNA) modified nucleoside (cMe); d=2′-deoxynucleosides.

TABLE 1

| | | | | Complementary |
|---|---|---|---|---|
| | | | | position to |
| | | | Sequence | human |
| SEQ | Con- | Nucleoside | of gapmer | AMANZI |
| ID NO. | figuration | Modification | compound | (SEQ ID NO. 1) |
| 2 | 5-10-5 | MMMMMddddddddddM | GAACTTGCAGGGG | 789-808 |
| | | MMMM | GCCGAGA | |
| 3 | 5-10-5 | MMMMMddddddddddM | CCCTCAATACAGA | 210-229 |
| | | MMMM | CAG GGAG | |
| 4 | 5-10-5 | MMMMMddddddddddM | ACTTTTCAGAGTTC | 481-500 |
| | | MMMM | ACCAAA | |
| 5 | 5-10-5 | MMMMMddddddddddM | TGGACTCTCATTCA | 638-657 |
| | | MMMM | TTCTAC | |
| 6 | 5-10-5 | MMMMMddddddddddM | CTTGCAGGGGGCC | 786-805 |
| | | MMMM | GAGATCG | |
| 7 | 5-10-5 | MMMMMddddddddddM | TTTCAGAGTTCACC | 478-497 |
| | | MMMM | AAAAAA | |
| 8 | 5-10-5 | MMMMMddddddddddM | AAAAAAAAAAAGT | 718-737 |
| | | MMMM | GTTATGA | |
| 9 | 5-10-5 | MMMMMddddddddddM | CCTGGACTCTCATT | 640-659 |
| | | MMMM | CATTCT | |
| 10 | 5-10-5 | MMMMMddddddddddM | AGCACGATACCTG | 1194-1213 |
| | | MMMM | GCACATA | |
| 11 | 5-10-5 | MMMMMddddddddddM | GAGCACGATACCT | 1195-1214 |
| | | MMMM | GGCACAT | |
| 12 | 5-10-5 | MMMMMddddddddddM | ATCTGGCATTGATC | 440-459 |
| | | MMMM | TGGTTC | |
| 13 | 5-10-5 | MMMMMddddddddddM | CATCTGGCATTGAT | 441-460 |
| | | MMMM | CTGGTT | |
| 14 | 5-10-5 | MMMMMddddddddddM | GCCAAATTAAAAA | 1324-1343 |
| | | MMMM | TCAGAAG | |
| 15 | 5-10-5 | MMMMMddddddddddM | GCGGAACTTGCAG | 792-811 |
| | | MMMM | GGGGCCG | |
| 16 | 5-10-5 | MMMMMddddddddddM | TAAAAATACAAAA | 886-905 |
| | | MMMM | AATTAGC | |
| 17 | 5-10-5 | MMMMMddddddddddM | ATACTAATTTGAAT | 1177-1196 |
| | | MMMM | AAAAAT | |
| 18 | 5-10-5 | MMMMMddddddddddM | TACTAATTTGAATA | 1176-1195 |
| | | MMMM | AAAATG | |
| 19 | 5-10-5 | MMMMMddddddddddM | CAGCTTTTAGATTT | 1406-1425 |
| | | MMMM | CCTGTG | |
| 20 | 5-10-5 | MMMMMddddddddddM | AGCTTTTAGATTTC | 1405-1424 |
| | | MMMM | CTGTGG | |
| 21 | 5-10-5 | MMMMMddddddddddM | AAGAAGTGAATGA | 123-142 |
| | | MMMM | AGAAAAG | |
| 22 | 5-10-5 | MMMMMddddddddddM | TACAAAGAAGTGA | 127-146 |
| | | MMMM | ATGAAGA | |
| 23 | 5-10-5 | MMMMMddddddddddM | ACAAAGAAGTGAA | 126-145 |
| | | MMMM | TGAAGAA | |
| 24 | 5-10-5 | MMMMMddddddddddM | GTTAAAGAGAAAC | 563-582 |
| | | MMMM | TGATAAC | |
| 25 | 5-10-5 | MMMMMddddddddddM | AGACCCACACCCT | 219-238 |
| | | MMMM | CAATACA | |
| 26 | 5-10-5 | MMMMMddddddddddM | ACTAATTTGAATA | 1175-1194 |
| | | MMMM | AAAATGC | |
| 27 | 5-10-5 | MMMMMddddddddddM | GCTTTTAGATTTCC | 1404-1423 |
| | | MMMM | TGTGGA | |
| 28 | 5-10-5 | MMMMMddddddddddM | CAAATTAAAAATC | 1322-1341 |
| | | MMMM | AGAAGGC | |
| 29 | 5-10-5 | MMMMMddddddddddM | CCAAATTAAAAAT | 1323-1342 |
| | | MMMM | CAGAAGG | |
| 30 | 5-10 -5 | MMMMMddddddddddM | TATCAAATTCACA | 1437-1456 |
| | | MMMM | GGAAACT | |
| 31 | 5-10-5 | MMMMMddddddddddM | TTATCAAATTCACA | 1438-1457 |
| | | MMMM | GGAAAC | |
| 32 | 5-10-5 | MMMMMddddddddddM | CTAATTTGAATAA | 1174-1193 |
| | | MMMM | AAATGCT | |
| 33 | 5-10-5 | MMMMMddddddddddM | CCAGGAGGAGCAA | 1048-1067 |
| | | MMMM | GGTTTCA | |
| 34 | 5-10-5 | MMMMMddddddddddM | CTCATCTGGCATTG | 443-462 |
| | | MMMM | ATCTGG | |
| 35 | 5-10-5 | MMMMMddddddddddM | TGTGACTGACTCCT | 1356-1375 |
| | | MMMM | GATATC | |
| 36 | 5-10-5 | MMMMMddddddddddM | GCTCCCCTGGGCA | 609-628 |
| | | MMMM | GTACCCT | |
| 37 | 5-10-5 | MMMMMddddddddddM | CCAAGAGATAGAG | 528-547 |
| | | MMMM | TCTCAGA | |
| 38 | 5-10-5 | MMMMMddddddddddM | GGAGGAGCAAGGT | 1045-1064 |
| | | MMMM | TTCAACT | |
| 39 | 5-10-5 | MMMMMddddddddddM | CTTATCAAATTCAC | 1439-1458 |
| | | MMMM | AGGAAA | |
| 40 | 3-10-3 | LLLddddddddddLLL | CATCAACTGCACA | 9-24 |
| | | | ACG | |
| 41 | 3-10-3 | LLLddddddddddLLL | ACATCAACTGCAC | 10-25 |
| | | | AAC | |
| 42 | 3-10-3 | LLLddddddddddLLL | GGACATCAACTGC | 12-27 |
| | | | ACA | |
| 43 | 3-10-3 | LLLddddddddddLLL | TGGACATCAACTG | 13-28 |
| | | | CAC | |
| 44 | 3-10-3 | LLLddddddddddLLL | TTGTCCCACATATA | 74-89 |
| | | | CT | |
| 45 | 3-10-3 | LLLddddddddddLLL | ACTTTGTCCCACAT | 77-92 |
| | | | AT | |
| 46 | 3-10-3 | LLLddddddddddLLL | CACTTTGTCCCACA | 78-93 |
| | | | TA | |
| 47 | 3-10-3 | LLLddddddddddLLL | CTAAACTTAAGTA | 142-157 |
| | | | CAA | |
| 48 | 3-10-3 | LLLddddddddddLLL | CCTAAACTTAAGT | 143-158 |
| | | | ACA | |
| 49 | 3-10-3 | LLLddddddddddLLL | TGTACCCATTTATC | 177-192 |
| | | | CA | |
| 50 | 3-10-3 | LLLddddddddddLLL | ACCGTATGTTCTCT | 324-339 |
| | | | GC | |
| 51 | 3-10-3 | LLLddddddddddLLL | GGGTTGTGTGAGC | 384-399 |
| | | | TTA | |
| 52 | 3-10-3 | LLLddddddddddLLL | AATCATTTTAGGCA | 464-479 |
| | | | GA | |
| 53 | 3-10-3 | LLLddddddddddLLL | AAATCATTTTAGGC | 465-480 |
| | | | AC | |
| 54 | 3-10-3 | LLLddddddddddLLL | AAAATCATTTTAG | 466-481 |
| | | | GCA | |
| 55 | 3-10-3 | LLLddddddddddLLL | ACTTTTCAGAGTTC | 485-500 |
| | | | AC | |
| 56 | 3-10-3 | LLLddddddddddLLL | CACTTTTCAGAGTT | 486-501 |
| | | | CA | |
| 57 | 3-10-3 | LLLddddddddddLLL | TTCCACTTTTCAGA | 489-504 |
| | | | GT | |
| 58 | 3-10-3 | LLLddddddddddLLL | ATTCCACTTTTCAG | 490-505 |
| | | | AG | |
| 59 | 3-10-3 | LLLddddddddddLLL | AATATTCCACTTTT | 493-508 |
| | | | CA | |
| 60 | 3-10-3 | LLLddddddddddLLL | CAATATTCCACTTT | 494-509 |
| | | | TC | |
| 61 | 3-10-3 | LLLddddddddddLLL | ACAATATTCCACTT | 495-510 |
| | | | TT | |
| 62 | 3-10-3 | LLLddddddddddLLL | TCTCAGATGGATAT | 519-534 |
| | | | TC | |
| 63 | 3-10-3 | LLLddddddddddLLL | GTCTCAGATGGAT | 520-535 |
| | | | ATT | |
| 64 | 3-10-3 | LLLddddddddddLLL | AGTCTCAGATGGA | 521-536 |
| | | | TAT | |
| 65 | 3-10-3 | LLLddddddddddLLL | GAGTCTCAGATGG | 522-537 |
| | | | ATA | |
| 66 | 3-10-3 | LLLddddddddddLLL | AGAGTCTCAGATG | 523-538 |
| | | | GAT | |
| 67 | 3-10-3 | LLLddddddddddLLL | TAGAGTCTCAGAT | 524-539 |
| | | | GGA | |
| 68 | 3-10-3 | LLLddddddddddLLL | ATAGAGTCTCAGA | 525-540 |
| | | | TGG | |
| 69 | 3-10-3 | LLLddddddddddLLL | ACCAAGAGATAGA | 533-548 |
| | | | GTC | |
| 70 | 3-10-3 | LLLddddddddddLLL | TACCAAGAGATAG | 534-549 |
| | | | AGT | |
| 71 | 3-10-3 | LLLddddddddddLLL | TTACCAAGAGATA | 535-550 |
| | | | GAG | |
| 72 | 3-10-3 | LLLddddddddddLLL | AACTGCACAACGA | 5-20 |
| | | | TTG | |
| 73 | 3-10-3 | LLLddddddddddLLL | CAACTGCACAACG | 6-21 |
| | | | ATT | |
| 74 | 3-10-3 | LLLddddddddddLLL | TCAACTGCACAAC | 7-22 |
| | | | GAT | |
| 75 | 3-10-3 | LLLddddddddddLLL | ATCAACTGCACAA | 8-23 |
| | | | CGA | |
| 76 | 3-10-3 | LLLddddddddddLLL | GCCCTTCATTGTAC | 186-201 |
| | | | CC | |
| 77 | 3-10-3 | LLLddddddddddLLL | GCTATTGGCCCTTC | 193-208 |
| | | | AT | |
| 78 | 3-10-3 | LLLddddddddddLLL | GGCTATTGGCCCTT | 194-209 |
| | | | CA | |
| 79 | 3-10-3 | LLLddddddddddLLL | AGGGCTATTGGCC | 196-211 |
| | | | CTT | |
| 80 | 3-10-3 | LLLddddddddddLLL | GAGGGCTATTGGC | 197-212 |
| | | | CCT | |
| 81 | 3-10-3 | LLLddddddddddLLL | GGAGGGCTATTGG | 198-213 |
| | | | CCC | |
| 82 | 3-10-3 | LLLddddddddddLLL | ACAGGGAGGGCTA | 202-217 |
| | | | TTG | |
| 83 | 3-10-3 | LLLddddddddddLLL | AGCACCCAAGGTA | 237-252 |
| | | | GAG | |
| 84 | 3-10-3 | LLLddddddddddLLL | CAGCACCCAAGGT | 238-253 |
| | | | AGA | |
| 85 | 3-10-3 | LLLddddddddddLLL | GAACCCTGCATAC | 335-350 |
| | | | CGT | |
| 86 | 3-10-3 | LLLddddddddddLLL | TGAACCCTGCATA | 336-351 |
| | | | CCG | |
| 87 | 3-10-3 | LLLddddddddddLLL | CCTGAACCCTGCAT | 338-353 |
| | | | AC | |
| 88 | 3-10-3 | LLLddddddddddLLL | CCCAACTCCGTCA | 355-370 |
| | | | GGA | |
| 89 | 3-10-3 | LLLddddddddddLLL | AGGGTTGCCCCAA | 363-378 |
| | | | CTC | |
| 90 | 3-10-3 | LLLddddddddddLLL | CCAGGGTTGCCCC | 365-380 |
| | | | AAC | |
| 91 | 3-10-3 | LLLddddddddddLLL | GATTGGCTAGGGT | 416-431 |
| | | | AAC | |
| 92 | 3-10-3 | LLLddddddddddLLL | AGATTGGCTAGGG | 417-432 |
| | | | TAA | |
| 93 | 3-10-3 | LLLddddddddddLLL | GAGATTGGCTAGG | 418-433 |
| | | | GTA | |
| 94 | 3-10-3 | LLLddddddddddLLL | ATGAGATTGGCTA | 420-435 |
| | | | GGG | |
| 95 | 3-10-3 | LLLddddddddddLLL | CATGAGATTGGCT | 421-436 |
| | | | AGG | |
| 96 | 3-10-3 | LLLddddddddddLLL | CCATGAGATTGGC | 422-437 |
| | | | TAG | |
| 97 | 3-10-3 | LLLddddddddddLLL | GGTATTACCAAGA | 539-554 |
| | | | GAT | |
| 98 | 3-10-3 | LLLddddddddddLLL | TTGGTATTACCAAG | 541-556 |
| | | | AG | |
| 99 | 3-10-3 | LLLddddddddddLLL | GTTGGTATTACCAA | 542-557 |
| | | | GA | |
| 100 | 3-10-3 | LLLddddddddddLLL | TGGTTGGTATTACC | 544-559 |
| | | | AA | |
| 101 | 3-10-3 | LLLddddddddddLLL | CTTGGTTGGTATTA | 546-561 |
| | | | CC | |
| 102 | 3-10-3 | LLLddddddddddLLL | TAACTCTTGGTTGG | 551-566 |
| | | | TA | |
| 103 | 3-10-3 | LLLddddddddddLLL | ATAACTCTTGGTTG | 552-567 |
| | | | GT | |
| 104 | 3-10-3 | LLLddddddddddLLL | GATAACTCTTGGTT | 553-568 |
| | | | GG | |
| 105 | 3-10-3 | LLLddddddddddLLL | CCAAGAAAGGTCA | 303-318 |
| | | | ATT | |
| 106 | 3-9-3 | MMMdddddddddMMM | AACACGTCTATAC | None |
| | | | GC | |
| 107 | 3-9-3 | LLLddddddddddLLL | AACACGTCTATAC | None |
| | | | GC | |

The gapmer compounds were analyzed for their effect on IL1B transcription in THP1 cells by quantitative real-time PCR. Similarly, the effect of the gapmer compounds on cytotoxicity and on Toll-like receptor (TLR) signaling activation were analyzed by assaying for the gene transcription of TNFRSF10B and secreted embryonic alkaline phosphatase (SEAP), respectively. Data are averages from four replicates in which THP1 cells were treated with the gapmer compounds of Table 1.

Table 2 shows the fold change of IL1B, TNFRSF10B, and SEAP gene expression in THP1 cells in the presence of the gapmer compounds in Table 1. Data are normalized by the expression of housekeeping gene RPL37A and represented as fold change relative to negative control (SEQ ID NO. 106 served as a negative control for MOE gapmers, and SEQ ID NO. 107 served as a negative control for LNA gapmers). An expression value <1.0 means that the transcription of that gene was inhibited, and an expression value >1.0 means that the transcription of that gene is induced. For example, a value of 0.25 means that gene transcription was inhibited by 75%.

TABLE 2

| SEQ | IL-1β | TNFRSF10B | SEAP |
|---|---|---|---|
| ID NO. | EXPRESSION | EXPRESSION | EXPRESSION |
| 2 | 0.832 | 0.809 | 0.479 |
| 3 | 0.918 | 0.913 | 0.861 |
| 4 | 0.814 | 0.886 | 1.039 |
| 5 | 0.732 | 0.965 | 0.906 |
| 6 | 0.538 | 0.978 | 0.736 |
| 7 | 1.726 | 1.100 | 1.442 |
| 8 | 0.709 | 1.362 | 0.953 |
| 9 | 1.442 | 1.140 | 1.451 |
| 10 | 0.937 | 0.941 | 1.231 |
| 11 | 1.230 | 1.453 | 1.702 |
| 12 | 1.800 | 1.531 | 2.212 |
| 13 | 1.286 | 1.486 | 1.725 |
| 14 | 0.671 | 1.140 | 0.901 |
| 15 | 0.972 | 1.079 | 1.041 |
| 16 | 1.330 | 1.370 | 1.814 |
| 17 | 0.925 | 0.853 | 0.943 |
| 18 | 1.399 | 1.112 | 1.219 |
| 19 | 1.519 | 0.830 | 1.128 |
| 20 | 1.014 | 0.779 | 0.911 |
| 21 | 0.843 | 0.684 | 0.707 |
| 22 | 1.191 | 0.832 | 1.234 |
| 23 | 0.971 | 0.804 | 0.803 |
| 24 | 1.114 | 0.982 | 1.273 |
| 25 | 0.793 | 0.860 | 0.994 |
| 26 | 0.726 | 1.001 | 0.927 |
| 27 | 1.458 | 1.103 | 1.311 |
| 28 | 0.967 | 0.961 | 0.972 |
| 29 | 1.105 | 0.951 | 1.088 |
| 30 | 1.750 | 1.429 | 1.777 |
| 31 | 1.130 | 1.193 | 1.533 |
| 32 | 1.465 | 1.353 | 1.309 |
| 33 | 1.585 | 1.383 | 1.191 |
| 34 | 1.294 | 1.234 | 1.285 |
| 35 | 1.440 | 1.144 | 1.550 |
| 36 | 1.081 | 0.734 | 0.881 |
| 37 | 1.154 | 0.979 | 1.144 |
| 38 | 1.116 | 0.972 | 0.830 |
| 39 | 1.470 | 0.834 | 1.058 |
| 40 | 1.188 | 2.530 | 1.159 |
| 41 | 2.112 | 3.741 | 1.630 |
| 42 | 3.289 | 4.755 | 2.352 |
| 43 | 2.140 | 3.236 | 1.655 |
| 44 | 2.127 | 4.026 | 2.033 |
| 45 | 2.059 | 2.550 | 1.530 |
| 46 | 2.791 | 3.779 | 2.114 |
| 47 | 2.149 | 3.444 | 1.371 |
| 48 | 1.945 | 2.863 | 1.472 |
| 49 | 1.347 | 1.493 | 0.871 |
| 50 | 1.251 | 0.818 | 0.918 |
| 51 | 2.408 | 1.466 | 1.982 |
| 52 | 0.897 | 1.503 | 0.906 |
| 53 | 1.054 | 0.984 | 1.094 |
| 54 | 0.938 | 0.763 | 0.713 |
| 55 | 1.104 | 0.944 | 0.763 |
| 56 | 0.920 | 0.603 | 0.747 |
| 57 | 1.285 | 0.625 | 0.926 |
| 58 | 1.150 | 0.639 | 0.922 |
| 59 | 0.817 | 0.083 | 0.971 |
| 60 | 0.938 | 2.096 | 0.908 |
| 61 | 1.450 | 1.884 | 1.014 |
| 62 | 1.524 | 2.706 | 1.409 |
| 63 | 2.016 | 2.490 | 1.335 |
| 64 | 2.303 | 3.289 | 1.329 |
| 65 | 1.844 | 1.737 | 1.155 |
| 66 | 2.366 | 2.899 | 1.345 |
| 67 | 2.788 | 2.535 | 1.218 |
| 68 | 1.897 | 2.514 | 1.218 |
| 69 | 2.117 | 0.990 | 1.223 |
| 70 | 1.054 | 0.510 | 0.956 |
| 71 | 1.073 | 0.300 | 1.057 |
| 72 | 0.719 | 0.947 | 0.608 |
| 73 | 0.856 | 0.753 | 0.784 |
| 74 | 0.645 | 1.025 | 0.877 |
| 75 | 0.984 | 0.754 | 1.204 |
| 76 | 0.322 | 0.269 | 0.149 |
| 77 | 1.185 | 0.899 | 0.820 |
| 78 | 1.954 | 1.866 | 1.195 |
| 79 | 1.775 | 2.482 | 1.064 |
| 80 | 1.508 | 1.548 | 0.855 |
| 81 | 1.805 | 1.440 | 0.832 |
| 82 | 2.189 | 1.930 | 1.351 |
| 83 | 2.953 | 1.528 | 0.836 |
| 84 | 2.968 | 1.895 | 0.922 |
| 85 | 1.607 | 1.407 | 0.787 |
| 86 | 1.593 | 1.412 | 0.972 |
| 87 | 1.991 | 1.261 | 1.404 |
| 88 | 0.868 | 1.033 | 0.817 |
| 89 | 1.064 | 0.793 | 0.936 |
| 90 | 0.810 | 0.535 | 0.843 |
| 91 | 1.095 | 0.372 | 0.691 |
| 92 | 1.215 | 0.584 | 0.724 |
| 93 | 1.214 | 0.451 | 0.952 |
| 94 | 1.151 | 0.960 | 1.892 |
| 95 | 1.792 | 0.624 | 1.253 |
| 96 | 1.778 | 0.557 | 2.115 |
| 97 | 1.082 | 0.632 | 1.461 |
| 98 | 1.638 | 1.107 | 0.694 |
| 99 | 1.152 | 1.001 | 0.803 |
| 100 | 1.860 | 1.741 | 1.331 |
| 101 | 2.893 | 1.443 | 1.380 |
| 102 | 2.016 | 1.312 | 1.016 |
| 103 | 2.358 | 1.430 | 1.045 |
| 104 | 2.323 | 1.515 | 1.283 |
| 105 | 1.410 | 0.974 | 1.328 |

Gapmer SEQ ID NOs. 41-47, 51, 63-64, 66-67, 69, 82-84, and 101-104 demonstrated at least two-fold upregulation of human IL1B expression in this assay. Among them, gapmer SEQ ID NO. 42 demonstrated more than three-fold upregulation of human IL1B expression in this assay.

Based on the screening data in Table 1 and 2, three regions (A, B, and C) in human AMANZI sequence (SEQ ID NO. 1) were found effectively targeted by gapmers SEQ ID NOs. 41-46, 63-68, 83-84, and 101-104. Table 3. provides the positions of AMANZI target regions (AMANZI Target Region A, B, and C) in human AMANZI sequence (SEQ ID NO. 1) and the average upregulation of IL1B by the gapmers targeted to individual regions.

TABLE 3

| | Target Region | | AVERAGE |
|---|---|---|---|
| AMANZI Target | in human AMANZI | SEQ | IL-1β |
| Region ID | (SEQ ID NO. 1) | ID NO. | EXPRESSION |
| A | 10-93 | 41-46 | 2.420 |
| B | 194-253 | 78, 81-84 | 2.374 |
| C | 519-568 | 63, 64, 66, 67, | 2.353 |
| | | 69, 101-104 | |

The gapmers targeted to region A, B, and C in human AMANZI (SEQ ID NO. 1) demonstrated more than 2.3-fold upregulation of IL1B gene expression on average. Gapmers targeting region A are selected from the group consisting of SEQ ID NOs. 41-46. Gapmers targeting region B are selected from the group consisting of SEQ ID NOs. 78, 81-84. Gapmers targeting region C are selected from the group consisting of SEQ ID NOs. 63, 64, 66, 67, 101-104.

Based on screening data in Table 3, a first supplemental group of chimeric phosphorothioate gapmer compounds that target the AMANZI target regions A, B, and C in human AMANZI (SEQ ID NO. 1) were synthesized. Table 4 provides the configuration, chemical modification, and sequence of the chimeric phosphorothioate gapmer compounds of the supplemental chimeric phosphorothioate gapmer compounds. Abbreviations of the nucleoside modification in Table 4: M=2′-methoxyethyl (2′-MOE) modified nucleoside; L=locked nucleic acid (LNA) modified nucleoside (cMe); d=2′-deoxynucleosides; 2′Md=2′OMe modified deoxynucleoside.

TABLE 4

| | | | | Complementary |
|---|---|---|---|---|
| | | | | position to |
| SEQ | Con- | Nucleoside | Sequence of | human AMANZI |
| ID NO. | figuration | Modification | gapmer compound | (SEQ ID NO. 1) |
| 108 | 5-10-5 | MMMMMddddddddddMM | GTGGACATCAACT | 10-29 |
| | | MMM | GCACAAC | |
| 109 | 6-8-6 | MMMMMMddddddddMMM | GTGGACATCAACT | 10-29 |
| | | MMM | GCACAAC | |
| 110 | 7-6-7 | MMMMMMMddddddMMM | GTGGACATCAACT | 10-29 |
| | | MMMM | GCACAAC | |
| 111 | 4-10-4 | MMMMddddddddddMMMM | TGGACATCAACTG | 11-28 |
| | | | CACAA | |
| 112 | 5-8-5 | MMMMMddddddddMMMM | TGGACATCAACTG | 11-28 |
| | | M | CACAA | |
| 113 | 4-8-4 | MMMMddddddddMMMM | GGACATCAACTGC | 12-27 |
| | | | ACA | |
| 114 | 5-6- 5 | MMMMMddddddMMMMM | GGACATCAACTGC | 12-27 |
| | | | ACA | |
| 115 | 3-10-4 | LLLddddddddddLLLL | TGGACATCAACTG | 12-28 |
| | | | CACA | |
| 116 | 5-8-4 | LLLLLddddddddLLLL | TGGACATCAACTG | 12-28 |
| | | | CACA | |
| 42 | 3-10-3 | LLLddddddddddLLL | GGACATCAACTGC | 12-27 |
| | | | ACA | |
| 117 | 5-7-4 | LLLLLdddddddLLLL | GGACATCAACTGC | 12-27 |
| | | | ACA | |
| 118 | 3-10-3 | LLLdd2′MddddddddLLL | GGACATCAACTGC | 12-27 |
| | | | ACA | |
| 119 | 5-10-5 | MMMMMddddddddddMM | CTCTTGGTTGGTAT | 544-563 |
| | | MMM | TA CCAA | |
| 120 | 6-8-6 | MMMMMMddddddddMMM | CTCTTGGTTGGTAT | 544-563 |
| | | MMM | TACCAA | |
| 121 | 7-6-7 | MMMMMMMddddddMMM | CTCTTGGTTGGTAT | 544-563 |
| | | MMMM | TACCAA | |
| 122 | 4-10-4 | MMMMddddddddddMMMM | CTCTTGGTTGGTAT | 546-563 |
| | | | TACC | |
| 123 | 5-8-5 | MMMMMddddddddMMMM | CTCTTGGTTGGTAT | 546-563 |
| | | M | TACC | |
| 124 | 4-8-4 | MMMMddddddddMMMM | CTTGGTTGGTATTA | 546-561 |
| | | | CC | |
| 125 | 5-6- 5 | MMMMMddddddMMMMM | CTTGGTTGGTATTA | 546-561 |
| | | | CC | |
| 126 | 3-10-4 | LLLddddddddddLLLL | CTCTTGGTTGGTAT | 547-563 |
| | | | TAC | |
| 127 | 5-8-4 | LLLLLddddddddLLLL | CTCTTGGTTGGTAT | 547-563 |
| | | | TAC | |
| 101 | 3-10-3 | LLLddddddddddLLL | CTTGGTTGGTATTA | 546-561 |
| | | | CC | |
| 128 | 5-7-4 | LLLLLdddddddLLLL | CTTGGTTGGTATTA | 546-561 |
| | | | CC | |
| 129 | 3-10-3 | LLLdd2′MddddddddLLL | CTTGGTTGGTATTA | 546-561 |
| | | | CC | |
| 130 | 5-10-5 | MMMMMddddddddddMM | ACAGCACCCAAGG | 235-254 |
| | | MMM | TAGAGAC | |
| 131 | 6-8-6 | MMMMMMddddddddMMM | ACAGCACCCAAGG | 235-254 |
| | | MMM | TAGAGAC | |
| 132 | 7-6-7 | MMMMMMMddddddMMM | ACAGCACCCAAGG | 235-254 |
| | | MMMM | TAGAGAC | |
| 133 | 4-10-4 | MMMMddddddddddMMMM | ACAGCACCCAAGG | 237-254 |
| | | | TAGAG | |
| 134 | 5-8-5 | MMMMMddddddddMMMM | ACAGCACCCAAGG | 237-254 |
| | | M | TAGAG | |
| 135 | 4-8-4 | MMMMddddddddMMMM | AGCACCCAAGGTA | 237-252 |
| | | | GAG | |
| 136 | 5-6-5 | MMMMMddddddMMMMM | AGCACCCAAGGTA | 237-252 |
| | | | GAG | |
| 137 | 3-10-4 | LLLddddddddddLLLL | ACAGCACCCAAGG | 239-254 |
| | | | TAG | |
| 138 | 5-8-4 | LLLLLddddddddLLLL | ACAGCACCCAAGG | 239-254 |
| | | | TAG | |
| 83 | 3-10-3 | LLLddddddddddLLL | AGCACCCAAGGTA | 237-252 |
| | | | GAG | |
| 139 | 5-7-4 | LLLLLdddddddLLLL | AGCACCCAAGGTA | 237-252 |
| | | | GAG | |
| 140 | 3-10-3 | LLLdd2′MddddddddLLL | AGCACCCAAGGTA | 237-252 |
| | | | GAG | |
| 141 | 5-10-5 | MMMMMddddddddddMM | TCCACTTTGTCCCA | 76-95 |
| | | MMM | CATATA | |
| 142 | 6-8-6 | MMMMMMddddddddMMM | TCCACTTTGTCCCA | 76-95 |
| | | MMM | CATATA | |
| 143 | 7-6-7 | MMMMMMMddddddMMM | TCCACTTTGTCCCA | 76-95 |
| | | MMMM | CATATA | |
| 144 | 4-10-4 | MMMMddddddddddMMMM | TCCACTTTGTCCCA | 78-95 |
| | | | CATA | |
| 145 | 5-8-5 | MMMMMddddddddMMMM | TCCACTTTGTCCCA | 78-95 |
| | | M | CATA | |
| 146 | 4-8-4 | MMMMddddddddMMMM | CACTTTGTCCCACA | 78-93 |
| | | | TA | |
| 147 | 5-6- 5 | MMMMMddddddMMMMM | CACTTTGTCCCACA | 78-93 |
| | | | TA | |
| 148 | 3-10-4 | LLLddddddddddLLLL | TCCACTTTGTCCCA | 79-95 |
| | | | CAT | |
| 149 | 5-8-4 | LLLLLddddddddLLLL | TCCACTTTGTCCCA | 79-95 |
| | | | CAT | |
| 46 | 3-10-3 | LLLddddddddddLLL | CACTTTGTCCCACA | 78-93 |
| | | | TA | |
| 150 | 5-7-4 | LLLLLdddddddLLLL | CACTTTGTCCCACA | 78-93 |
| | | | TA | |
| 151 | 3-10-3 | LLLdd2′MddddddddLLL | CACTTTGTCCCACA | 78-93 |
| | | | TA | |
| 152 | 5-10-5 | MMMMMddddddddddMMM | GATAGAGTCTCAG | 522-541 |
| | | MM | ATGGATA | |
| 153 | 6-8-6 | MMMMMMddddddddMMM | GATAGAGTCTCAG | 522-541 |
| | | MMM | ATGGATA | |
| 154 | 7-6-7 | MMMMMMMddddddMMM | GATAGAGTCTCAG | 522-541 |
| | | MMMM | ATGGATA | |
| 155 | 4-10-4 | MMMMddddddddddMMMM | GATAGAGTCTCAG | 524-541 |
| | | | ATGGA | |
| 156 | 5-8-5 | MMMMMddddddddMMMM | GATAGAGTCTCAG | 524-541 |
| | | M | ATGGA | |
| 157 | 4-8-4 | MMMMddddddddMMMM | TAGAGTCTCAGAT | 524-539 |
| | | | GGA | |
| 158 | 5-6-5 | MMMMMddddddMMMMM | TAGAGTCTCAGAT | 524-539 |
| | | | GGA | |
| 159 | 3-10-4 | LLLddddddddddLLLL | GATAGAGTCTCAG | 525-541 |
| | | | ATGG | |
| 160 | 5-8-4 | LLLLLddddddddLLLL | GATAGAGTCTCAG | 525-541 |
| | | | ATGG | |
| 67 | 3-10-3 | LLLddddddddddLLL | TAGAGTCTCAGAT | 524-539 |
| | | | GGA | |
| 161 | 5-7-4 | LLLLLdddddddLLLL | TAGAGTCTCAGAT | 524-539 |
| | | | GGA | |
| 162 | 3-10-3 | LLLdd2′MddddddddLLL | TAGAGTCTCAGAT | 524-539 |
| | | | GGA | |

The first supplemental chimeric phosphorothioate gapmer compounds in Table 4. were analyzed for their effect on IL1B, TNFRSF10B, and SEAP transcription in THP1 cells by quantitative real-time PCR. Table 5 shows the induction of IL1B, TNFRSF10B, and SEAP gene expression by the first supplemental chimeric phosphorothioate gapmers SEQ ID NOs 42, 46, 67, 83, 101 that target AMANZI target regions (region A, B, and C) in human AMANZI sequence (SEQ ID NO. 1). Data were normalized by the expression of housekeeping gene RPL37A and represented as fold change relative to negative control (SEQ ID NO. 106 served as a negative control for MOE gapmers, and SEQ ID NO. 107 served as a negative control for LNA gapmers). An expression value <1.0 means that the transcription of that gene was inhibited, and an expression value >1.0 means that the transcription of that gene is induced. For example, a value of 0.25 means that gene transcription was inhibited by 75%.

TABLE 5

| SEQ | IL-1β | TNFRSF10B | SEAP |
|---|---|---|---|
| ID NO. | EXPRESSION | EXPRESSION | EXPRESSION |
| 108 | 2.145 | 1.817 | 1.547 |
| 109 | 1.887 | 1.511 | 1.034 |
| 110 | 1.630 | 1.484 | 1.541 |
| 111 | 2.624 | 1.497 | 1.865 |
| 112 | 1.636 | 1.177 | 1.229 |
| 113 | 1.621 | 1.296 | 1.436 |
| 114 | 2.028 | 1.331 | 1.303 |
| 115 | 0.832 | 1.636 | 1.258 |
| 116 | 1.602 | 1.387 | 1.015 |
| 42 | 1.003 | 1.372 | 1.218 |
| 117 | 1.332 | 1.432 | 1.054 |
| 118 | 2.185 | 1.646 | 1.448 |
| 119 | 0.952 | 2.415 | 1.450 |
| 120 | 1.140 | 0.953 | 1.809 |
| 121 | 0.620 | 1.554 | 1.488 |
| 122 | 1.196 | 1.188 | 1.858 |
| 123 | 1.021 | 0.834 | 1.224 |
| 124 | 1.128 | 0.676 | 1.195 |
| 125 | 2.742 | 1.330 | 2.226 |
| 126 | 3.941 | 1.584 | 1.722 |
| 127 | 2.327 | 0.015 | 1.962 |
| 101 | 2.321 | 0.080 | 2.031 |
| 128 | 2.206 | 3.445 | 1.814 |
| 129 | 3.465 | 2.001 | 1.364 |
| 130 | 2.046 | 1.810 | 1.401 |
| 131 | 2.150 | 2.108 | 1.561 |
| 132 | 1.177 | 1.254 | 1.438 |
| 133 | 1.860 | 1.922 | 1.583 |
| 134 | 0.609 | 1.028 | 1.170 |
| 135 | 1.459 | 1.846 | 1.932 |
| 136 | 0.529 | 0.127 | 1.215 |
| 137 | 0.818 | 0.035 | 1.019 |
| 138 | 0.888 | 0.170 | 1.095 |
| 83 | 1.282 | 0.215 | 1.132 |
| 139 | 0.811 | 0.230 | 0.890 |
| 140 | 0.745 | 0.140 | 0.961 |
| 141 | 0.749 | 1.028 | 0.514 |
| 142 | 0.939 | 1.137 | 0.749 |
| 143 | 1.056 | 1.222 | 0.830 |
| 144 | 1.176 | 1.300 | 0.894 |
| 145 | 1.074 | 1.127 | 0.792 |
| 146 | 1.040 | 1.011 | 0.762 |
| 147 | 0.776 | 1.040 | 0.707 |
| 148 | 2.118 | 1.263 | 1.190 |
| 149 | 1.438 | 0.937 | 0.852 |
| 46 | 1.305 | 1.192 | 0.732 |
| 150 | 2.132 | 0.979 | 1.330 |
| 151 | 2.162 | 0.884 | 0.953 |
| 152 | 1.217 | 1.294 | 1.119 |
| 153 | 1.101 | 1.030 | 0.889 |
| 154 | 1.021 | 0.775 | 1.120 |
| 155 | 1.137 | 1.010 | 0.901 |
| 156 | 1.024 | 1.231 | 1.014 |
| 157 | 0.917 | 0.918 | 0.805 |
| 158 | 1.140 | 0.950 | 0.980 |
| 159 | 1.592 | 1.250 | 1.153 |
| 160 | 1.636 | 1.656 | 3.616 |
| 67 | 1.402 | 1.026 | 1.546 |
| 161 | 1.480 | 1.326 | 2.915 |
| 162 | 2.559 | 1.981 | 1.548 |

Based on screening data in Table 4 and 5, another set of LNA gapmer compounds (second supplemental) that target human AMANZI sequence (SEQ ID NO. 1) were synthesized and tested. Table 6 provides the configuration, nucleoside modification, and sequence of the second supplemental gapmer compounds used in the present examples and embodiments described herein. Abbreviations of the nucleoside modification in Table 6: L=locked nucleic acid (LNA) modified nucleoside (cMe); d=2′-deoxynucleosides.

TABLE 6

| | | | | Complementary |
|---|---|---|---|---|
| | | | | position to |
| | | | | human |
| SEQ ID | Con- | Nucleoside | Sequence of | AMANZI |
| NO. | figuration | Modification | gapmer compound | (SEQ ID NO. 1) |
| 163 | 3-10-3 | LLLddddddddddLLL | TCATAACCTGGACTCT | 650-665 |
| 164 | 3-10-3 | LLLddddddddddLLL | TCCCCTGGGCAGTACC | 611-626 |
| 165 | 3-10-3 | LLLddddddddddLLL | TACAAATGGAGGGCT | 625-640 |
| | | | C | |
| 166 | 3-10-3 | LLLddddddddddLLL | TCTACAAATGGAGGG | 627-642 |
| | | | C | |
| 167 | 3-10-3 | LLLddddddddddLLL | ACTCTCATTCATTCTA | 639-654 |
| 168 | 3-10-3 | LLLddddddddddLLL | TGGACTCTCATTCATT | 642-657 |
| 169 | 3-10-3 | LLLddddddddddLLL | ATAACCTGGACTCTCA | 648-663 |
| 170 | 3-10-3 | LLLddddddddddLLL | GTGTTCCTACACTCCA | 674-689 |
| 171 | 3-10-3 | LLLddddddddddLLL | GGTGTTCCTACACTCC | 675-690 |
| 172 | 3-10-3 | LLLddddddddddLLL | AGGGTGTTCCTACACT | 677-692 |
| 173 | 3-10-3 | LLLddddddddddLLL | CAAAGGAGGGTGTTC | 683-698 |
| | | | C | |
| 174 | 3-10-3 | LLLddddddddddLLL | GCAGACCTGTCAAAG | 701-716 |
| | | | A | |
| 175 | 3-10-3 | LLLddddddddddLLL | GATGCAGACCTGTCAA | 704-719 |
| 176 | 3-10-3 | LLLddddddddddLLL | TGATGCAGACCTGTCA | 705-720 |
| 177 | 3-10-3 | LLLddddddddddLLL | AGTGTTATGATGCAGA | 712-727 |
| 178 | 3-10-3 | LLLddddddddddLLL | GGGGGCCGAGATCGT | 784-799 |
| | | | G | |
| 179 | 3-10-3 | LLLddddddddddLLL | CTAGGGTAACAGCAC | 410-425 |
| | | | C | |
| 180 | 3-10-3 | LLLddddddddddLLL | TAGGGTAACAGCACCT | 409-424 |
| 181 | 3-10-3 | LLLddddddddddLLL | GCTAGGGTAACAGCA | 411-426 |
| | | | C | |
| 182 | 3-10-3 | LLLddddddddddLLL | CAGATGGATATTCTTA | 516-531 |
| 183 | 3-10-3 | LLLddddddddddLLL | TGGCTAGGGTAACAG | 413-428 |
| | | | C | |
| 184 | 3-10-3 | LLLddddddddddLLL | TTGGCTAGGGTAACAG | 414-429 |
| 185 | 3-10-3 | LLLddddddddddLLL | ATTGGCTAGGGTAACA | 415-430 |
| 186 | 3-10-3 | LLLddddddddddLLL | ATTACCAAGAGATAG | 536-551 |
| | | | A | |
| 187 | 3-10-3 | LLLddddddddddLLL | GATCTGGTTCATCCAT | 434-449 |
| 188 | 3-10-3 | LLLddddddddddLLL | TTGATCTGGTTCATCC | 436-451 |
| 189 | 3-10-3 | LLLddddddddddLLL | ATTGATCTGGTTCATC | 437-452 |
| 190 | 3-10-3 | LLLddddddddddLLL | CTCATCTGGCATTGAT | 447-462 |
| 191 | 3-10-3 | LLLddddddddddLLL | GAGTTCACCAAAAAA | 477-492 |
| | | | T | |
| 192 | 3-10-3 | LLLddddddddddLLL | CTCTTGGTTGGTATTA | 548-563 |
| 193 | 3-10-3 | LLLddddddddddLLL | ACTCTTGGTTGGTATT | 549-564 |
| 194 | 3-10-3 | LLLddddddddddLLL | AACTGATAACTCTTGG | 557-572 |
| 195 | 3-10-3 | LLLddddddddddLLL | AAACTGATAACTCTTG | 558-573 |
| 196 | 3-10-3 | LLLddddddddddLLL | GAGAAACTGATAACT | 561-576 |
| | | | C | |
| 197 | 3-10-3 | LLLddddddddddLLL | AGAGAAACTGATAAC | 562-577 |
| | | | T | |
| 198 | 3-10-3 | LLLddddddddddLLL | CGGTTAAAGAGAAAC | 569-584 |
| | | | T | |
| 199 | 3-10-3 | LLLddddddddddLLL | TCGGTTAAAGAGAAA | 570-585 |
| | | | C | |
| 200 | 3-10-3 | LLLddddddddddLLL | CTCGGTTAAAGAGAA | 571-586 |
| | | | A | |
| 201 | 3-10-3 | LLLddddddddddLLL | TGTCTCGGTTAAAGAG | 574-589 |
| 202 | 3-10-3 | LLLddddddddddLLL | GTGTCTCGGTTAAAGA | 575-590 |
| 203 | 3-10-3 | LLLddddddddddLLL | GGTGTCTCGGTTAAAG | 576-591 |
| 204 | 3-10-3 | LLLddddddddddLLL | TGGTGTCTCGGTTAAA | 577-592 |
| 205 | 3-10-3 | LLLddddddddddLLL | CTGGTGTCTCGGTTAA | 578-593 |
| 206 | 3-10-3 | LLLddddddddddLLL | TTGCTGGTGTCTCGGT | 581-596 |
| 207 | 3-10-3 | LLLddddddddddLLL | TACCCTGGAGCAGGC | 599-614 |
| | | | A | |
| 208 | 3-10-3 | LLLddddddddddLLL | GTACCCTGGAGCAGG | 600-615 |
| | | | C | |
| 209 | 3-10-3 | LLLddddddddddLLL | TCCGTCAGGAGCCTGA | 349-364 |
| 210 | 3-10-3 | LLLddddddddddLLL | GTCAGGAGCCTGAAC | 346-361 |
| | | | C | |
| 211 | 3-10-3 | LLLddddddddddLLL | ACTCCGTCAGGAGCCT | 351-366 |
| 212 | 3-10-3 | LLLddddddddddLLL | AACTCCGTCAGGAGCC | 352-367 |
| 213 | 3-10-3 | LLLddddddddddLLL | CAACTCCGTCAGGAGC | 353-368 |
| 214 | 3-10-3 | LLLddddddddddLLL | CGTCAGGAGCCTGAA | 347-362 |
| | | | C | |
| 215 | 3-10-3 | LLLddddddddddLLL | CCAACTCCGTCAGGAG | 354-369 |
| 216 | 3-10-3 | LLLddddddddddLLL | GCCCCAACTCCGTCAG | 357-372 |
| 217 | 3-10-3 | LLLddddddddddLLL | TGCCCCAACTCCGTCA | 358-373 |
| 218 | 3-10-3 | LLLddddddddddLLL | TTGCCCCAACTCCGTC | 359-374 |
| 219 | 3-10-3 | LLLddddddddddLLL | GTTGCCCCAACTCCGT | 360-375 |
| 220 | 3-10-3 | LLLddddddddddLLL | GGTTGCCCCAACTCCG | 361-376 |
| 221 | 3-10-3 | LLLddddddddddLLL | GGGTTGCCCCAACTCC | 362-377 |
| 222 | 3-10-3 | LLLddddddddddLLL | TCCAGGGTTGCCCCAA | 366-381 |
| 223 | 3-10-3 | LLLddddddddddLLL | GTGAGCTTATCTCCAG | 377-392 |
| 224 | 3-10-3 | LLLddddddddddLLL | GTGTGAGCTTATCTCC | 379-394 |
| 225 | 3-10-3 | LLLddddddddddLLL | TGTGTGAGCTTATCTC | 380-395 |
| 226 | 3-10-3 | LLLddddddddddLLL | TTGTGTGAGCTTATCT | 381-396 |
| 227 | 3-10-3 | LLLddddddddddLLL | GTTGTGTGAGCTTATC | 382-397 |
| 228 | 3-10-3 | LLLddddddddddLLL | GGTTGTGTGAGCTTAT | 383-398 |
| 229 | 3-10-3 | LLLddddddddddLLL | TTGCAGGGTTGTGTGA | 389-404 |
| 230 | 3-10-3 | LLLddddddddddLLL | CTTGCAGGGTTGTGTG | 390-405 |
| 231 | 3-10-3 | LLLddddddddddLLL | GGTCTTGCAGGGTTGT | 393-408 |
| 232 | 3-10-3 | LLLddddddddddLLL | GCACAACGATTGTCAG | 1-16 |
| 233 | 3-10-3 | LLLddddddddddLLL | TGCACAACGATTGTCA | 2-17 |
| 234 | 3-10-3 | LLLddddddddddLLL | CTGCACAACGATTGTC | 3-18 |
| 235 | 3-10-3 | LLLddddddddddLLL | GTGGGAAGATTCCTAA | 154-169 |
| 236 | 3-10-3 | LLLddddddddddLLL | TGTCCCACATATACTA | 73-88 |
| 237 | 3-10-3 | LLLddddddddddLLL | TATCCATCTGTAAGTG | 167-182 |
| 238 | 3-10-3 | LLLddddddddddLLL | AATGTGGACATCAACT | 17-32 |

The second supplemental LNA gapmer compounds in Table 6. were similarly analyzed for their effect on IL1B, TNFRSF10B, and SEAP transcription in THP1 cells by quantitative real-time PCR. Table 7 shows the induction of IL1B gene expression by second supplemental LNA gapmers SEQ ID NOs 163-238 that target human AMANZI sequence (SEQ ID NO. 1). Data were normalized by the expression of housekeeping gene RPL37A and represented as fold change relative to negative control (SEQ ID NO. 106 served as a negative control for MOE gapmers, and SEQ ID NO. 107 served as a negative control for LNA gapmers). An expression value <1.0 means that the transcription of that gene was inhibited, and an expression value >1.0 means that the transcription of that gene is induced.

Gapmer SEQ ID NOs. 173, 201-208, 226, 223-229, and 235 demonstrated at least three-fold upregulation of human IL1B expression in this assay.

TABLE 7

| SEQ ID | IL-1β | TNFRSF10B | SEAP |
|---|---|---|---|
| NO. | EXPRESSION | EXPRESSION | EXPRESSION |
| 163 | 0.615 | 0.803 | 0.545 |
| 164 | 1.637 | 0.392 | 0.793 |
| 165 | 1.577 | 1.491 | 0.922 |
| 166 | 2.310 | 1.523 | 1.045 |
| 167 | 1.785 | 1.110 | 0.827 |
| 168 | 1.518 | 1.333 | 0.780 |
| 169 | 1.525 | 1.371 | 0.829 |
| 170 | 1.894 | 0.922 | 0.882 |
| 171 | 1.627 | 0.959 | 1.092 |
| 172 | 1.439 | 0.740 | 1.655 |
| 173 | 3.378 | 1.286 | 2.818 |
| 174 | 0.859 | 1.108 | 0.745 |
| 175 | 0.843 | 1.108 | 0.892 |
| 176 | 1.454 | 1.072 | 1.067 |
| 177 | 1.249 | 0.734 | 1.048 |
| 178 | 2.049 | 0.189 | 0.660 |
| 179 | 0.840 | 0.727 | 0.703 |
| 180 | 0.715 | 0.653 | 0.653 |
| 181 | 0.802 | 0.371 | 0.896 |
| 182 | 1.777 | 0.296 | 2.569 |
| 183 | 0.766 | 1.338 | 0.810 |
| 184 | 1.642 | 1.412 | 1.052 |
| 185 | 1.048 | 1.545 | 0.954 |
| 186 | 1.411 | 1.192 | 0.763 |
| 187 | 2.697 | 1.424 | 1.549 |
| 188 | 1.919 | 1.995 | 1.326 |
| 189 | 1.512 | 1.347 | 0.997 |
| 190 | 1.339 | 1.150 | 1.002 |
| 191 | 1.696 | 0.680 | 0.900 |
| 192 | 1.741 | 0.152 | 1.298 |
| 193 | 0.990 | 0.033 | 1.088 |
| 194 | 1.348 | 0.011 | 0.498 |
| 195 | 0.885 | 1.117 | 0.962 |
| 196 | 0.769 | 0.898 | 0.846 |
| 197 | 0.862 | 0.830 | 0.712 |
| 198 | 0.692 | 0.962 | 0.712 |
| 199 | 1.158 | 1.031 | 0.921 |
| 200 | 1.210 | 0.624 | 1.179 |
| 201 | 3.363 | 1.592 | 1.904 |
| 202 | 3.680 | 1.503 | 1.719 |
| 203 | 4.634 | 1.367 | 1.894 |
| 204 | 4.793 | 1.049 | 1.652 |
| 205 | 5.123 | 2.049 | 2.301 |
| 206 | 3.417 | 1.860 | 1.998 |
| 207 | 7.625 | 2.269 | 3.603 |
| 208 | 4.835 | 1.840 | 2.442 |
| 209 | 2.294 | 1.673 | 1.596 |
| 210 | 2.180 | 1.408 | 1.739 |
| 211 | 1.362 | 1.217 | 1.287 |
| 212 | 0.936 | 1.117 | 1.093 |
| 213 | 1.623 | 1.090 | 1.468 |
| 214 | 1.461 | 0.876 | 1.419 |
| 215 | 1.006 | 1.238 | 1.190 |
| 216 | 3.469 | 0.417 | 1.543 |
| 217 | 1.211 | 0.308 | 1.254 |
| 218 | 1.203 | 0.086 | 1.206 |
| 219 | 1.562 | 0.059 | 1.090 |
| 220 | 0.762 | 0.025 | 1.223 |
| 221 | 1.738 | 1.801 | 1.357 |
| 222 | 1.917 | 1.459 | 1.693 |
| 223 | 3.353 | 1.501 | 1.804 |
| 224 | 3.361 | 1.420 | 1.835 |
| 225 | 3.973 | 1.260 | 1.969 |
| 226 | 4.560 | 0.900 | 2.044 |
| 227 | 3.166 | 0.907 | 1.802 |
| 228 | 4.370 | 0.989 | 1.869 |
| 229 | 3.772 | 1.239 | 2.104 |
| 230 | 2.829 | 0.797 | 2.054 |
| 231 | 2.773 | 0.445 | 1.970 |
| 232 | 1.758 | 0.944 | 1.941 |
| 233 | 2.073 | 1.454 | 1.496 |
| 234 | 1.792 | 1.284 | 1.194 |
| 235 | 3.877 | 1.131 | 1.458 |
| 236 | 1.766 | 0.851 | 1.155 |
| 237 | 1.834 | 0.888 | 1.161 |
| 238 | 1.162 | 0.683 | 1.039 |

Based on the screening data in Table 6 and 7, another two regions in human AMANZI sequence (SEQ ID NO. 1) (Regions D and E) were found effectively targeted by gapmers SEQ ID NOs. 201-208 and SEQ ID NOs. 223-229. Table 8. provides the positions of AMANZI target regions (AMANZI Target Regions D and E) and the average upregulation of IL1B by the gapmers targeted to individual regions.

TABLE 8

| | AMANZI Target Region in | | |
|---|---|---|---|
| AMANZI | human AMANZI | SEQ ID | AVERAGE IL-1β |
| Region ID | (SEQ ID NO. 1) | NO. | EXPRESSION |
| D | 377-404 | 223-229 | 3.794 |
| E | 574-615 | 201-208 | 4.684 |

The gapmers targeted to regions D and E in human AMANZI (SEQ ID NO. 1) demonstrated more than 3.5-fold upregulation of IL1B gene expression on average. Gapmers targeting region D are selected from the group consisting of SEQ ID NOs. 228-234. Gapmers targeting region B are selected from the group consisting of SEQ ID NOs. 206-213.

Table 5 shows results of AMANZI inhibition and corresponding gene activation. A value less than 1, represents inhibition.

THP1 monocytes are heterozygotes for rs16944 (G>A) at the 268th nucleotide of human AMANZI sequence (SEQ ID NO. 1). To understand the allele specific effect on the induction of IL-1B transcripts in the trained immunity, allele-specific MOE gapmer compounds targeting the region spanning rs16944 were synthesized. Table 6 provides the configuration, nucleoside modification, and sequence of gapmer compounds. Abbreviations of the nucleoside modification in Table 9: M=2′-methoxyethyl (2′-MOE) modified nucleoside; d=2′-deoxynucleosides.

TABLE 9

| | | | | Complementary | |
|---|---|---|---|---|---|
| | | | | position to | |
| | | | | human | Targeted |
| SEQ | Con- | | Nucleobase | AMANZI | allele of |
| ID NO. | figuration | Chemistry | Sequence | (SEQ ID NO. 1) | rs16944 |
| 239 | 5-10-5 | MMMMMdddddddd | AGCTCCTGAGG | 252-271 | A |
| | | ddMMMMM | CAGAG AACA | | |
| 240 | 5-10-5 | MMMMMdddddddd | AGCTCCCGAGG | 252-271 | G |
| | | ddMMMMM | CAGAG AACA | | |

### Example 2: AMANZI Inhibition in β-Glucan-Trained Monocyte Cell Line, THP1

Trained immunity was induced in THP1 monocytes as follows; THP1 monocytes were grown with culture medium RPMI 1640 (Gibco) supplemented with 10% fetal bovine serum (FBS) (Gibco), GlutaMAX (Gibco) and 50 nM 2-Mercaptoethanol (Gibco). THP1 monocytes were then seeded in flat-bottom 96-well plates (Corning, NY, USA) and incubated with the culture medium added with or without 2 μg/ml of β-glucan, together with 10 μM of test gapmer SEQ ID NO. 239, 240, or MOE negative control gapmer (SEQ ID NO. 106) for 24 hours at 37° C. Cells were washed once with 200 μL warm PBS and incubated for 5 days in culture medium. Medium was changed once on day 3. On day 6, cells were restimulated with either 200 μl RPMI or 10 ng/ml of LPS (Serotype 055: B5; Sigma) for another 24 h, and the gene expression of IL1B was quantified by RT-qPCR.

Table 10. shows the gene expression of IL1B when THP1 monocytes were trained in the presence of gapmer compounds SEQ ID NO. 239, 240, or MOE negative control gapmer (SEQ ID NO. 106). Data are normalized by the expression of housekeeping gene RPL37A and represented as fold change relative to MOE negative control gapmer (SEQ ID NO. 106).

TABLE 10

| SEQ ID NO. | IL-1β EXPRESSION |
|---|---|
| 239 | 5.8 |
| 240 | 0.9 |
| 106 | 1 |

Table 10. shows a differential effect of gapmer compounds SEQ ID NO. 239 and 240 upon the gene expression of IL1B in the trained immunity of THP1 monocytes, in which gapmer compound SEQ ID NO. 239 induced 5.8-fold IL1B transcripts while gapmer compound SEQ ID NO. 240 slightly inhibited IL1B transcription in comparison to negative control gapmer (SEQ ID NO. 107).

### Example 3: AMANZI Inhibition in LPS Stimulated CD14 Positive Monocytes

Percoll monocytes from healthy donors (n=3) were seeded in serum-free RPMI (Gibco) containing 1% Pen/Strep (Gibco) at 1×106 in a 24 well plate (Sarstedt) and incubated at 37° C. at 5% CO2 for 1 hour. Plated cells were washed with serum-free RPMI and treated with RPMI (ss)+10% pooled human serum (ss) containing SEQ 24 or SEQ ID 107 gapmer compounds and incubated at 37° C. at 5% CO2 for overnight. Each treatment was conducted in duplicate. Plated cells were then treated with 10 ng/ml Ultrapure LPS (Invitrogen) and incubated at 37° C. at 5% CO2 for overnight. Gapmer-treated cells were then centrifuged at 400 rpm for 5 min at room temperature and RNA was isolated using the MagMAX RNA Total RNA Isolation KIt (Thermo Fisher Scientific) in accordance with the manufacturer's recommended protocols. Total RNA was quantified using the NanoDrop® (Thermo Fisher Scientific) manufacturer's recommended protocols. Total RNA underwent a reverse transcriptase reaction (RT) using iScript cDNA synthesis kit (Bio-Rad) according to manufacturer's recommended reagents and protocols to produce complementary DNA used as substrate for quantitative real-time polymerase chain reaction (RT-qPCR). Quantification of target RNA levels was conducted by RT-qPCR with SsoAdvanced Universal SYBR Green according to manufacturer's recommended protocols, using a CFX Real-time PCR detection system (Bio Rad). The target quantities obtained by RT-qPCR were normalized using the expression of a stably expressed housekeeping gene, RPL37A.

Table 11 displays the fold change of IL1B gene expression in LPS-treated monocytes (n=3 donors) in the presence of the gapmer compounds (SEQ ID NO 42). Data were normalized by the expression of housekeeping gene RPL37A and represented as fold change relative to LPS treated monocytes with control gapmer compound (SEQ ID 107). An expression value <1.0 means that the transcription of that gene was inhibited, and an expression value >1.0 means that the transcription of that gene was induced. For example, a value of 0.25 means that gene transcription was inhibited by 75%.

TABLE 11

| | AVERAGE SEQ ID | IL-1β |
|---|---|---|
| SEQ ID NO. | NO: 1 EXPRESSION | EXPRESSION |
| 42 | 0.47 | 3.359 |
| 107 | 1 | 1 |

### Example 4: AMANZI Inhibition in PBMCs Treated with LPS and R848

Peripheral blood mononuclear cells (PBMCs) from healthy donors (n=4), with an average age of 56.3±14.9 years, were purchased cryopreserved from CTL Europe. Cryopreserved PBMCs were thawed in pre-warmed Wash Media (RPMI (Dutch modification)+20% FBS+2 mM GlutaMAX+1 mM Sodium pyruvate+1% Pen/Strep). PBMCs were then washed in pre-warmed Wash Media three times before centrifugation in pre-warmed Wash Media at 500 rpm for 10 min at room temperature. Centrifugation was repeated twice before resuspending cells in pre-warmed Culture Media (RPMI (Dutch modification)+10% FBS+2 mM GlutaMAX+1 mM Sodium pyruvate+1% Pen/Strep). Next, cells were counted and seeded at approximately 1×10⁵ cells and were added into each well of a 96-well U-bottom tissue culture plate (CellSTAR) containing a TLR cocktail containing 1 μg/mL Ultrapure LPS (Invitrogen) and 5 μg/mL Resiquimod (Invitrogen) with or without gapmer compounds (SEQ ID 111). Gapmer compounds were added at a low dose (5 μM) or high dose (30 μM). Each treatment was conducted in duplicate and plated cells were incubated at 37° C. at 5% CO₂ for 48 hours. Plated cells were then centrifuged at 400 rpm for 5 min at room temperature and RNA was isolated using the MagMAX RNA Total RNA Isolation Kit (Thermo Fisher Scientific) in accordance with the manufacturer's recommended protocols. Total RNA was then quantified using the NanoDrop® (Thermo Fisher Scientific) manufacturer's recommended protocols. Total RNA then underwent a reverse transcriptase reaction (RT) using an iScript cDNA synthesis kit (Bio-Rad), according to the manufacturer's recommended reagents and protocols, to produce complementary DNA. The complementary DNA was used as substrate for quantitative real-time polymerase chain reaction (RT-qPCR) using SsoAdvanced Universal SYBR Green in accordance with manufacturer's recommended protocols. Quantification of target RNA levels was conducted by RT-qPCR using a CFX Real-time PCR detection system (Bio-Rad). The target quantities obtained by RT-qPCR were normalized using the expression of a stably expressed housekeeping gene, RPL37A.

Table 12 displays the fold change of IL1B gene expression in TLR-stimulated PBMCs in the presence of the gapmer compounds (SEQ ID 111). Data were normalized by expression of housekeeping gene RPL37A and represented a fold change relative to PBMCs treated with TLR cocktails without gapmer compounds. An expression value <1.0 means that the transcription of that gene was inhibited, and an expression value >1.0 means that the transcription of that gene was induced. For example, a value of 0.25 means that gene transcription was inhibited by 75%.

TABLE 12

| SEQ ID | DONOR | GAPMER | IL1B |
|---|---|---|---|
| NO. | ID | DOSAGE | EXPRESSION |
| 111 | 1 | LOW | 1.60 |
| 111 | 1 | HIGH | 0.80 |
| 111 | 2 | LOW | 1.72 |
| 111 | 2 | HIGH | 1.13 |
| 111 | 3 | LOW | 1.15 |
| 111 | 3 | HIGH | 2.13 |
| 111 | 4 | LOW | 1.47 |
| 111 | 4 | HIGH | 3.27 |

### Example 5: IL-1B eRNA Inhibition in Prostatic Adenocarcinoma Cell Line, PC-3

PC-3 cells (ATCC) were seeded at 6×10⁴ per well in a 24-well plate (Sarstedt) and RPMI 1640 containing GlutaMAX (Gibco), 10% FBS (Gibco) and 1% Pen/Strep (Gibco) and gapmer compound SEQ ID 42 were added. Gapmer compounds were added at 200 nM. Each treatment was conducted in duplicate. Plated cells were incubated at 37° C. at 5% CO₂ for 8 hours. Gapmer-treated cells were then centrifuged at 400 rpm for 5 min at room temperature and RNA was isolated using the MagMAX RNA miRVana Total RNA Isolation Kit (Thermo Fisher Scientific) in accordance with the manufacturer's recommended protocols. Total RNA was quantified using the NanoDrop® (Thermo Fisher Scientific) manufacturer's recommended protocols. Total RNA underwent a reverse transcriptase reaction (RT) using an iScript cDNA synthesis kit (Bio-Rad) according to the manufacturer's recommended reagents and protocols to produce complementary DNA to be used as substrate for quantitative real-time polymerase chain reaction (RT-qPCR). Quantification of target RNA levels was conducted by RT-qPCR with SsoAdvanced Universal SYBR Green according to the manufacturer's recommended protocols using a CFX Real-time PCR detection system (Bio-Rad). The target quantities obtained by RT-qPCR were normalized using the expression of a stably expressed housekeeping gene, RPL37A.

Table 13 displays the fold change of IL1B gene expression in PC-3 cells in the presence of the gapmer compounds. Data was normalized by the expression of housekeeping gene RPL37A and represented as fold change relative to untreated PC-3 cells. An expression value <1.0 means that the transcription of that gene was inhibited, and an expression value >1.0 means that the transcription of that gene was induced. For example, a value of 0.25 means that gene transcription was inhibited by 75%.

TABLE 13

| SEQ ID NO. | IL-1β EXPRESSION |
|---|---|
| 42 | 1.51 |
| 107 | 1 |

### Example 6: IL-1B eRNA Inhibition in TNF-α Treated Pancreatic Cancer Cell Line, MIA PaCa2

MIA PaCa-2 cells (ATCC) were seeded in RPMI 1640+10% FBS+1% Pen/Strep at 1×10⁶ cells per well in a 24-well plate (Sarstedt) and incubated at 37° C. at 5% CO₂ for 24 hours. Plated cells were treated with media containing 10 ng/ml TNF and incubated at 37° C. at 5% CO₂ for 24 hours. Cells were then treated with media containing SEQ ID 111 or control SEQ ID 106 and incubated at 37° C. at 5% CO₂ for 24 hours. Each treatment was conducted in triplicate. Gapmer-treated cells were then centrifuged at 400 rpm for 5 min at room temperature and RNA was isolated using the MagMAX RNA Total RNA Isolation Kit (Thermo Fisher Scientific) in accordanding to manufacturer's protocols. Total RNA was quantified using the NanoDrop® (Thermo Fisher Scientific) manufacturer's recommended protocols. Total RNA underwent a reverse transcriptase reaction (RT) using iScript cDNA synthesis kit (Bio-Rad), according to the manufacturer's recommended reagents and protocols, to produce complementary DNA. Complementary DNA was used as a substrate for quantitative real-time polymerase chain reaction (RT-qPCR). Quantification of target RNA levels was conducted by RT-qPCR with SsoAdvanced Universal SYBR Green according to manufacturer's recommended protocols using a CFX Real-time PCR detection system (Bio-Rad). The target quantities obtained by RT-qPCR were normalized using the expression of a stably expressed housekeeping gene, RPL37A.

Table 14 displays the fold change of IL1B gene expression in TNFα-treated MIA PaCa-2 cells in the presence of the gapmer compounds (SEQ ID NO 111). Data were normalized by the expression of housekeeping gene RPL37A and represented as fold change relative to TNFα treated cells with control gamper compounds (SEQ ID 106). An expression value <1.0 means that the transcription of that gene is inhibited, and an expression value >1.0 means that the transcription of that gene is induced. For example, a value of 0.25 means that gene transcription was inhibited by 75%.

TABLE 14

| SEQ ID NO. | IL-1β EXPRESSION |
|---|---|
| 111 | 2.05 |
| 106 | 1 |

This disclosure provides for the following embodiments:

A1. A gapmer type of ASO (antisense oligonucleotide) that can inhibit AMANZI transcriptional activity, comprising (a) from about 14 to about 25 nucleotide bases; (b) a 3′ wing region (3′ to 5′) having from 3 to 7 chemically modified RNA bases; (c) a gap region having from at least 8 DNA bases to no more than 19 DNA bases; and (d) a 5′ wing region (3′ to 5′) having from 3 to 7 chemically modified RNA bases; wherein the gapmer is substantially complementary to a 14-25 base region on AMANZI (SEQ ID NO. 1).

A2. The gapmer compound that specifically inhibits AMANZI of embodiment 1, wherein the gapmer nucleotide bases are each linked by phosphorothiolate (P═S) internucleotide bonds throughout the gapmer; and wherein the modified nucleotide base modifications are selected from the group consisting of 2′-methoxyethyl (MOE) nucleotides, locked nucleic acid nucleotides (LNA), and combinations thereof.

A3. A gapmer type of ASO (antisense oligonucleotide) that can inhibit AMANZI transcriptional activity, comprising (a) from about 14 to about 25 nucleotide bases; (b) a 3′ wing region (3′ to 5′) having from 3 to 7 chemically modified RNA bases; (c) a gap region having from at least 8 DNA bases to no more than 19 DNA bases; and (d) a 5′ wing region (3′ to 5′) having from 3 to 7 chemically modified RNA bases; wherein the gapmer is substantially complementary to Region A of AMANZI (SEQ ID NO. 1 bases 10 to 93).

A4. The gapmer type of ASO that can inhibit AMANZI of embodiment 3, wherein the gap region comprises a 10-nucleotide DNA sequence from nucleotide 5 to nucleotide 15 from any of SEQ ID NOs. 41-46 or an 8 mer fragment thereof.

A5. The gapmer type of ASO that can inhibit AMANZI of embodiment 3, wherein the gapmer nucleotides are each linked by phosphorothiolate (P═S) internucleotide bonds throughout the gapmer; and wherein the modified nucleotide base modifications are selected from the group consisting of 2′-methoxyethyl (MOE) nucleotides, locked nucleic acid nucleotides (LNA), and combinations thereof.

A6. The gapmer type of ASO that can inhibit AMANZI of embodiment 3, wherein Region A of AMANZI is SEQ ID NO. 1 base 10 to base 93.

A7. The gapmer type of ASO that can inhibit AMANZI of embodiment 3, wherein the gapmer type of ASO is selected from the group consisting of SEQ ID NO. 41, SEQ ID NO. 42, SEQ ID NO. 43, SEQ ID NO. 44, SEQ ID NO. 45, SEQ ID NO. 46, and combinations thereof.

A8. The gapmer type of ASO that can inhibit AMANZI of embodiment 7, wherein the gapmer type of ASO is SEQ ID NO. 42.

A9. A gapmer type of ASO (antisense oligonucleotide) that can inhibit AMANZI transcriptional activity, wherein the gapmer comprises (a) from about 14 to about 25 nucleotide bases; (b) a 3′ wing region (3′ to 5′) having from 3 to 7 chemically modified RNA bases; (c) a gap region having from at least 8 DNA bases to no more than 19 DNA bases; and (d) a 5′ wing region (3′ to 5′) having from 3 to 7 chemically modified RNA bases; wherein the gapmer is substantially complementary to Region B of AMANZI (SEQ ID NO. 1 bases 194 to 253).

A10. The gapmer type of ASO that can inhibit AMANZI of embodiment 9, wherein the gap region comprises a 10-nucleotide DNA sequence from nucleotide 5 to nucleotide 15 from any of SEQ ID NO. 78, SEQ ID NO. 81, SEQ ID NO. 82, SEQ ID NO. 83, SEQ ID NO. 84, and combinations thereof, or an 8 mer fragment thereof.

A11. The gapmer type of ASO that can inhibit AMANZI of embodiment 9, wherein the gapmer nucleotides are each linked by phosphorothiolate (P═S) internucleotide bonds throughout the gapmer; and wherein the modified nucleotide base modifications are selected from the group consisting of 2′-methoxyethyl (MOE) nucleotides, locked nucleic acid nucleotides (LNA), and combinations thereof.

A12. The gapmer type of ASO that can inhibit AMANZI of embodiment 9, wherein Region B of AMANZI is SEQ ID NO. 1 base 194 to base 253.

A13. The gapmer type of ASO that can inhibit AMANZI of embodiment 9, wherein the gapmer type of ASO is selected from the group consisting of SEQ ID NO. 78, SEQ ID NO. 81, SEQ ID NO. 82, SEQ ID NO. 83, SEQ ID NO. 84, and combinations thereof.

A14. The gapmer type of ASO that can inhibit AMANZI of embodiment 13, wherein the gapmer type of ASO is SEQ ID NO. 84.

A15. A gapmer type of ASO (antisense oligonucleotide) that can inhibit AMANZI transcriptional activity, wherein the gapmer comprises (a) from about 14 to about 25 nucleotide bases; (b) a 3′ wing region (3′ to 5′) having from 3 to 7 chemically modified RNA bases; (c) a gap region having from at least 8 DNA bases to no more than 19 DNA bases; and (d) a 5′ wing region (3′ to 5′) having from 3 to 7 chemically modified RNA bases; wherein the gapmer is substantially complementary to Region C of AMANZI (SEQ ID NO. 1 bases 519 to 568).

A16. The gapmer type of ASO that can inhibit AMANZI of embodiment 15, wherein the gap region comprises a 10-nucleotide DNA sequence from nucleotide 5 to nucleotide 15 from any of SEQ ID NO. 63, SEQ ID NO. 64, SEQ ID NO. 66, SEQ ID NO. 67, SEQ ID NO. 69, SEQ ID NO. 101, SEQ ID NO. 102, SEQ ID NO. 103, SEQ ID NO. 104, and combinations thereof, or an 8 mer fragment thereof.

A17. The gapmer type of ASO that can inhibit AMANZI of embodiment 15, wherein the gapmer nucleotides are each linked by phosphorothiolate (P═S) internucleotide bonds throughout the gapmer; and wherein the modified nucleotide base modifications are selected from the group consisting of 2′-methoxyethyl (MOE) nucleotides, locked nucleic acid nucleotides (LNA), and combinations thereof.

A18. The gapmer type of ASO that can inhibit AMANZI of embodiment 15, wherein Region C of AMANZI is SEQ ID NO. 1 base 519 to base 568.

A19. The gapmer type of ASO that can inhibit AMANZI of embodiment 15, wherein the gapmer type of ASO is selected from the group consisting of SEQ ID NO. 63, SEQ ID NO. 64, SEQ ID NO. 66, SEQ ID NO. 67, SEQ ID NO. 69, SEQ ID NO. 101, SEQ ID NO. 102, SEQ ID NO. 103, SEQ ID NO. 104, and combinations thereof.

A20. The gapmer type of ASO that can inhibit AMANZI of embodiment 15, wherein the gapmer type of ASO is SEQ ID NO. 101.

A21. A gapmer type of ASO (antisense oligonucleotide) that can inhibit AMANZI transcriptional activity, wherein the gapmer comprises (a) from about 14 to about 25 nucleotide bases; (b) a 3′ wing region (3′ to 5′) having from 3 to 7 chemically modified RNA bases; (c) a gap region having from at least 8 DNA bases to no more than 19 DNA bases; and (d) a 5′ wing region (3′ to 5′) having from 3 to 7 chemically modified RNA bases; wherein the gapmer is substantially complementary to substantially complementary Region D of AMANZI (SEQ ID NO. 1 bases 377 to 404).

A22. The gapmer type of ASO that can inhibit AMANZI of embodiment 21, wherein the gap region comprises a 10-nucleotide DNA sequence from nucleotide 5 to nucleotide 15 from any of SEQ ID NO. 223, SEQ ID NO. 224, SEQ ID NO. 225, SEQ ID NO. 226, SEQ ID NO. 227, SEQ ID NO. 228, SEQ ID NO. 229, and combinations thereof, or an 8 mer fragment thereof.

A23. The gapmer type of ASO that can inhibit AMANZI of embodiment 21, wherein the gapmer nucleotides are each linked by phosphorothiolate (P═S) internucleotide bonds throughout the gapmer; and wherein the modified nucleotide base modifications are selected from the group consisting of 2′-methoxyethyl (MOE) nucleotides, locked nucleic acid nucleotides (LNA), and combinations thereof.

A24. The gapmer type of ASO that can inhibit AMANZI of embodiment 21, wherein Region D of AMANZI is SEQ ID NO. 1 base 377 to base 404.

A25. The gapmer type of ASO that can inhibit AMANZI of embodiment 21, wherein the gapmer type of ASO is selected from the group consisting of SEQ ID NO. 223, SEQ ID NO. 224, SEQ ID NO. 225, SEQ ID NO. 226, SEQ ID NO. 227, SEQ ID NO. 228, SEQ ID NO. 229, and combinations thereof.

A26. The gapmer type of ASO that can inhibit AMANZI of embodiment 21, wherein the gapmer type of ASO is SEQ ID NO. 226.

A27. A gapmer type of ASO (antisense oligonucleotide) that can inhibit AMANZI transcriptional activity, wherein the gapmer comprises (a) from about 14 to about 25 nucleotide bases; (b) a 3′ wing region (3′ to 5′) having from 3 to 7 chemically modified RNA bases; (c) a gap region having from at least 8 DNA bases to no more than 19 DNA bases; and (d) a 5′ wing region (3′ to 5′) having from 3 to 7 chemically modified RNA bases; wherein the gapmer is substantially complementary to substantially complementary Region E of AMANZI (SEQ ID NO. 1 bases 574 to 615).

A28. The gapmer type of ASO that can inhibit AMANZI of embodiment 27, wherein the gap region comprises a 10-nucleotide DNA sequence from nucleotide 5 to nucleotide 15 from any of SEQ ID NO. 201, SEQ ID NO. 202, SEQ ID NO. 203, SEQ ID NO. 204, SEQ ID NO. 205, SEQ ID NO. 206, SEQ ID NO. 207, SEQ ID NO. 208, and combinations thereof, or an 8 mer fragment thereof.

A29. The gapmer type of ASO that can inhibit AMANZI of embodiment 27, wherein the gapmer nucleotides are each linked by phosphorothiolate (P═S) internucleotide bonds throughout the gapmer; and wherein the modified nucleotide base modifications are selected from the group consisting of 2′-methoxyethyl (MOE) nucleotides, locked nucleic acid nucleotides (LNA), and combinations thereof.

A30. The gapmer type of ASO that can inhibit AMANZI of embodiment 27, wherein Region E of AMANZI is SEQ ID NO. 1 base 574 to base 615.

A31. The gapmer type of ASO that can inhibit AMANZI of embodiment 27, wherein the gapmer type of ASO is selected from the group consisting of SEQ ID NO. 201, SEQ ID NO. 202, SEQ ID NO. 203, SEQ ID NO. 204, SEQ ID NO. 205, SEQ ID NO. 206, SEQ ID NO. 207, SEQ ID NO. 208, and combinations thereof.

A32. The gapmer type of ASO that can inhibit AMANZI of embodiment 27, wherein the gapmer type of ASO is SEQ ID NO. 207.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents. Reference to any applications, patents and publications in this specification is not, and should not be taken as, an acknowledgment or any form of suggestion that they constitute valid prior art or form part of the common general knowledge in any country in the world.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” may be replaced with either of the other two terms in the specification. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants. Furthermore, titles, headings, or the like are provided to enhance the reader's comprehension of this document, and should not be read as limiting the scope of the present invention. Any examples of aspects, embodiments or components of the invention referred to herein are to be considered non-limiting.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

We claim:

1. A gapmer type of ASO (antisense oligonucleotide) that can inhibit AMANZI transcriptional activity, comprising (a) from about 14 to about 25 nucleotide bases; (b) a 3′ wing region (3′ to 5′) having from 3 to 7 chemically modified RNA bases; (c) a gap region having from at least 8 DNA bases to no more than 19 DNA bases; and (d) a 5′ wing region (3′ to 5′) having from 3 to 7 chemically modified RNA bases; wherein the gapmer is substantially complementary to a 14-25 base region on AMANZI (SEQ ID NO. 1).

2. The gapmer compound that specifically inhibits AMANZI of claim 1, wherein the gapmer nucleotide bases are each linked by phosphorothiolate (P═S) internucleotide bonds throughout the gapmer; and wherein the modified nucleotide base modifications are selected from the group consisting of 2′-methoxyethyl (MOE) nucleotides, locked nucleic acid nucleotides (LNA), and combinations thereof.

3. A gapmer type of ASO (antisense oligonucleotide) that can inhibit AMANZI transcriptional activity, comprising (a) from about 14 to about 25 nucleotide bases; (b) a 3′ wing region (3′ to 5′) having from 3 to 7 chemically modified RNA bases; (c) a gap region having from at least 8 DNA bases to no more than 19 DNA bases; and (d) a 5′ wing region (3′ to 5′) having from 3 to 7 chemically modified RNA bases; wherein the gapmer is substantially complementary to substantially complementary to Region A of AMANZI (SEQ ID NO. 1 bases 12 to 92).

4. The gapmer type of ASO that can inhibit AMANZI of claim 3, wherein the gap region comprises a 10-nucleotide DNA sequence from nucleotide 5 to nucleotide 15 from any of SEQ ID NOs. 2-105 or an 8 mer fragment thereof.

5. The gapmer type of ASO that can inhibit AMANZI of claim 3, wherein the gapmer nucleotides are each linked by phosphorothiolate (P═S) internucleotide bonds throughout the gapmer; and wherein the modified nucleotide base modifications are selected from the group consisting of 2′-methoxyethyl (MOE) nucleotides, locked nucleic acid nucleotides (LNA), and combinations thereof.

6. The gapmer type of ASO that can inhibit AMANZI of claim 3, wherein Region A of AMANZI is SEQ ID NO. 1 base 12 to base 27.

7. The gapmer type of ASO that can inhibit AMANZI of claim 3, wherein the gapmer type of ASO is selected from the group consisting of SEQ ID NO. 41, SEQ ID NO. 42, SEQ ID NO. 43, SEQ ID NO. 44, SEQ ID NO. 45, SEQ ID NO. 46, and combinations thereof.

8. The gapmer type of ASO that can inhibit AMANZI of claim 7, wherein the gapmer type of ASO is SEQ ID NO. 42.

9. The gapmer type of ASO that can inhibit AMANZI of claim 3, wherein (a) from about 14 to about 25 nucleotide bases; (b) a 3′ wing region (3′ to 5′) having from 3 to 7 chemically modified RNA bases; (c) a gap region having from at least 8 DNA bases to no more than 19 DNA bases; and (d) a 5′ wing region (3′ to 5′) having from 3 to 7 chemically modified RNA bases; wherein the gapmer is substantially complementary to substantially complementary Region B of AMANZI (SEQ ID NO. 1 bases 519 to 568).

10. The gapmer type of ASO that can inhibit AMANZI of claim 9, wherein the gap region comprises a 10-nucleotide DNA sequence from nucleotide 5 to nucleotide 15 from any of SEQ ID NO. 63, SEQ ID NO. 63, SEQ ID NO. 64, SEQ ID NO. 66, SEQ ID NO. 67, SEQ ID NO. 101, SEQ ID NO. 102, SEQ ID NO. 103, SEQ ID NO. 104, and combinations thereof, or an 8 mer fragment thereof.

11. The gapmer type of ASO that can inhibit AMANZI of claim 9, wherein the gapmer nucleotides are each linked by phosphorothiolate (P═S) internucleotide bonds throughout the gapmer; and wherein the modified nucleotide base modifications are selected from the group consisting of 2′-methoxyethyl (MOE) nucleotides, locked nucleic acid nucleotides (LNA), and combinations thereof.

12. The gapmer type of ASO that can inhibit AMANZI of claim 9, wherein the gapmer type of ASO is selected from the group consisting of SEQ ID NO. 63, SEQ ID NO. 63, SEQ ID NO. 64, SEQ ID NO. 66, SEQ ID NO. 67, SEQ ID NO. 101, SEQ ID NO. 102, SEQ ID NO. 103, SEQ ID NO. 104, and combinations thereof.

13. The gapmer type of ASO that can inhibit AMANZI of claim 12, wherein the gapmer type of ASO is SEQ ID NO. 101.

14. A gapmer type of ASO (antisense oligonucleotide) that can inhibit AMANZI transcriptional activity, wherein the gapmer comprises (a) from about 14 to about 25 nucleotide bases; (b) a 3′ wing region (3′ to 5′) having from 3 to 7 chemically modified RNA bases; (c) a gap region having from at least 8 DNA bases to no more than 19 DNA bases; and (d) a 5′ wing region (3′ to 5′) having from 3 to 7 chemically modified RNA bases; wherein the gapmer is substantially complementary to substantially complementary Region D of AMANZI (SEQ ID NO. 1 bases 377 to 404).

15. The gapmer type of ASO that can inhibit AMANZI of claim 14, wherein the gap region comprises a 10-nucleotide DNA sequence from nucleotide 5 to nucleotide 15 from any of SEQ ID NOs 228-234, and combinations thereof, or an 8 mer fragment thereof.

16. The gapmer type of ASO that can inhibit AMANZI of claim 14, wherein the gapmer nucleotides are each linked by phosphorothiolate (P═S) internucleotide bonds throughout the gapmer; and wherein the modified nucleotide base modifications are selected from the group consisting of 2′-methoxyethyl (MOE) nucleotides, locked nucleic acid nucleotides (LNA), and combinations thereof.

17. A gapmer type of ASO (antisense oligonucleotide) that can inhibit AMANZI transcriptional activity, wherein the gapmer comprises (a) from about 14 to about 25 nucleotide bases; (b) a 3′ wing region (3′ to 5′) having from 3 to 7 chemically modified RNA bases; (c) a gap region having from at least 8 DNA bases to no more than 19 DNA bases; and (d) a 5′ wing region (3′ to 5′) having from 3 to 7 chemically modified RNA bases; wherein the gapmer is substantially complementary to substantially complementary Region E of AMANZI (SEQ ID NO. 1 bases 574 to 615).

18. The gapmer type of ASO that can inhibit AMANZI of claim 17, wherein the gap region comprises a 10-nucleotide DNA sequence from nucleotide 5 to nucleotide 15 from any of SEQ ID NOs. 206-215, and combinations thereof, or an 8 mer fragment thereof.

19. The gapmer type of ASO that can inhibit AMANZI of claim 17, wherein the gapmer nucleotides are each linked by phosphorothiolate (P═S) internucleotide bonds throughout the gapmer; and wherein the modified nucleotide base modifications are selected from the group consisting of 2′-methoxyethyl (MOE) nucleotides, locked nucleic acid nucleotides (LNA), and combinations thereof.
